SEQUENCE LISTING (52 sequences)

SEQ ID NO: 1 - DNA, 74970 bp, Homo sapiens
gccgaggcaggccgatcacgaggtcaggagatcgagaccatcctggctaacatggtgaaaccctgtctctactaaaaatacaaaaaaattagccgggcatggtggagggcgcctgtagtcccagctacttgggaggctgaggcaggagaatggcgtgaaccctggatgcggaggttgcagtgagctgagatcacgccactgcactccagcctgggcgagagagtgagactctgtctcaaaaaaaaaaaaaaaagaagagtaagattcctctgcccctcaaaagctgatagttcagtagtatataaagtaatacttccagtatgatgtaatggtgctgtaatgtaggcatgctcaaggtgtagcggtggcacaaaatacttttccttagtgaactttccagaagtaaaaggaagaagctagtatgactagagtctggagtgactggggcagagagaatttggggagaacagaacaggacaggtggttggtccagatcacagagcttttcaggccagcaataggatggcaataggaagtcactgaaggattttttttttttttttttttttttgaggcagagtctcactctgttgcccaggctggagtgcactgttgcgatctcatctcactgcaacctctgccgcccaggttcaagcaattctgcctcagcttcccaagtagctgggattacaggtgcccaccacaacacccggctaatttttgtatttttagtagagatggggtttcaccatgttggataggctggtcttgaactcctgacctcaggtgatccacccgcctcggcctcccaaagtactgggattacaggcatgagccattgcacccggccatcactgaaggattttgaagtcagagttgtgtaatcatttaagaatccctgccaatgtaagtcccctgaaggtgggactgtttgtgtttttcatggttatagtcccagggcctgatatgcagttattcttgttaaacaaaagaaaatcttgtgagatttgcatttttggaagatcactgacagcaccatgaattagacatattgagactggaatcagaagaccaattcaaagatccttggactagagtacacaagataccaagagggcttgaactaagtagtagcaatgaagacaagggcaagagagagttaggagtggatttatagcatttgaggactgattggaataaggggtgacgtggagaagttgtgggaagagaataaaaaatgattcccagatacttggtgctatgatctgaatgtttgtatctttccaaaattcatttgttgaaatgtagtctccagtgtgttggtttaacagatggggcctttgggagatgattagatcataaggccaagccttgtgactgagattagtgcccttatgcagagaggaagcttgtttgccccttttccccttccaccatgtgaggacaaaaaaatgcaccatctatgaggaatgggccctcaccagacactaaatccgttagagccttgatcttggacttcctagcctgtaaatttattacagatctgtaataaacttctgtttataaattactctaagatatcttgttaaagcgccccaaaaagactaagaccttcgacttcattaattcatttgaaagacatcaactacaatttaaaaaaacaaaacaaaaacagaaaaggtggtttgcaatttttttcacttcatgagactataatttatttccctgggtaggccacacaaatccttaagaacatggactttcccccatacaaggctgtgaaccgtgcacacagagatgctcgataaatggtaccgaatcctctccttcagcacctggtcctctctggcagtctccattctcttacctctcactgccccctctacttctctgaacctgcagtccccaaagtcaccaatggtctccatagcaacaaaatttagtgggctcttatacgaccttatattgcttgacttcttggcagcaactgacactgactaccccttgaagcatcccctgcccaggccttccacaatgtcacactctcttgcttctcctccctcctccttggttattgcttctcctatcctcacggctcagctctagccctcttctcaatcacttccttttccctaaaatgattttattcactcctatggtttcagttacttttttttttttttttttttgagacacagtttcactctgttgtccaggctggagtgcaatagcacgatcttggctcactgcaacctctacctcccggttcaagtgattctcctgcctcagcctcctgagtagctgggattataggcatgagccaccacgcccggctaatttttgtatttttgtagagatgaggtttcaccatgttggccagcctggtcttgaactcctggcctcaagagatctgcctgcctcagcctcccaaagtgctgggattacaggcatgcaccaccacaatggccttagctatctgttttttaatgacaattcctcattgagttcaaagcagcctcctgaacttcagactcggtatccaaccattactcaacataattactcaaatgtccatagatatttcaaattcagtatctccaaaatgtaaactctatcttattctcttcccaaaaccttgctgctcctcccatgtttctgctctcaataccactcctgccacagtggctgcccaggtcacaaacccagaagtcaccttcacatttctgtctcctaccttccccaatccctcattgtggtaggagttattaagaaattattttaggcagatagagaggaaaaggggtccttgaaaagtttttgtctcttttgaagcagctcctgagacgtttcctgtctagcataaaagccctgacttttagacccgagctggcaacgtttgatatgcaaatgccggcagttagaaactgggtccaccaacacggtgattcccaccatcgtcctcttgcccttgttctcacaagtgcctggcaacatggccacccccacatatccccacatgagtagaacatcatagcgccctgcatttgcatattaaaaggctagggtggaatggccagttttattagggggctacatgaatgacatgcgtggtcaaaccagtcccctgagccctatgcaaatcagacaccgcctcctgcatcctcctcctataactggctggtatctcccacgcactctgggtctcctctctcagctttggagcacccatccctctgtctctgtacaggggagctgcttctttctttcttctcccttccttcttgcttattaaactctccgctccttaaaaccactccacgtttgtccatgtcgttttatgtaattcgactcaagacgaaaaaggctagtattcctccactcggtatcatcaccacattctgttgagtctctttacgtccaaagaatctctcaaaatgggctatttccatctttaagacactctcttaaaccaccatcgtctctcccaagattccttcaacagcaactgcttttttctgcagccagtgatctttctaaaaggcaaacatgaccacatgtcttcttttaaaatatttatatagctccccacatcctttaaaaggtacactttggccaggtgcggtggctcacgcctgtaattccaggactttgggaggccaaggcaggtggatcacctgaggctgggggttcgagaccagcctggccaacatggtacaacctcttctctactaagaatacaaaaatagttggacttcgtggtgcacgcctgtaatcccagctgtttgggaggctgaggcaagagaatcacttgaacccaggaagcagaagttgcagtgagcggggagattgtgccactgcactccagcctgggggacagagagagggccgtctcaaaaaaaaaaaaaaaaaaagtacaccttaatctggtttcaaggtgcaagatctgcacctatccttccatttaagtttcatcaaaccgctgctacctccccgtctcccacacaatttatgggacttctaagttccctctaaagggtccgaacacctacactggtaacaagccacctggatttgaatcctggcaagacaacttactatctgaccttggacattgtgctgttcttaacctctccgtgcctcggtttcctcatgtgtatgaataacatcaacacctacatcaaagtttgctgtattaaatttgataatatatgcaaagcatttagaaaagtgcctagctcatagaaagccttatgtaaatattaactatcattttttttcttttttggggtggtgggggaggggtttcgcttttgttgcccaggctggcgtgcaatggcacgatctcggctcaccacaacctcggcctcccgggttcaagcgattctcctgcctcagcctcccgagtacctgggattacaggcatgcaccaccacgcccgggtaattttgtatttttagtggagacggggtttctccatgttggtcaggctgatctcaaactcccgacctcaggtgatccgcccgcctcggcctcccaaagtgctgggattacaagcgtgaagcacctcgcccggcctagctatcatttttatacaagtgctgggttttgggagaatgtaatgatggcttttttcttactaaactttcagtgcaggaggaggagaaagaaagtaaatagttatatgaacacagtagaaagtcaaagtggaaaacaaaaagaacatagaacccaggtgagcggtccagacctccccccagaaacctaagaatccatagaaatgggtgggaagcggagaagatcctccagacagcaggtggcgatgtagcatcccccagaaggcccgctaacagaagctaggaggacgcgctaccaaggtcacgtgtccccggcgttcactcgctcttcgcttcacgacactcgcatcctcacgggtgattggtctgcgtgcggcacgtgggcggggtaccggggcgggccggggaggggcggggtgggcggaggagaggggcagggggcggagctggagggggtggttcggcgtgggggccgttggctccagacaaataaacatggagtccatcttccacgagaaagtgagtgtccgcgttcggtggggagctgtctgccgcgcggtggcgggcgtggagcgcggcatcaccgcctctcggagggctgggtggggcccgagtcgcccccatgccgatctcgcccggcgaggggcgacgccgcagcctcccgcctcctcggctcgaggaggggagcatcacctacgcccctacttcccccgcggcccccgccctgggagccgggagggagtatgggcggggccgggggcgtctcgggacacgggagtggggtggcgcccagtgggtttgcttctgcctttctccgtcactttccatcgcttttcggaggattccttcacccctccccaatccttccctctccctagggtctagctagagtcatctctgggacacctccctcaacccctcctaccctaatcctggcagaattaacttttcctcctccggactgctcaattctatattggagtcttccctacacgtagatctttggggtcttgttcgtgtctttcccctgcactaggtccgcgagcctcccgagggaggagaccttggctcgcccactgtagggcctgacatttaggaagtgaagtaggaaacccggcgtgcccctaaacagggaagtcgtcacaagagtttttattacgggatgtttgggtttggtttcttttggtactcccatctttccggagcaggcggccagctttgtttttaggtattaggagtggactgggatgattttgttgtagtctgcctagcctgctgtccctttaactcttccgtgaccatgcacttgaagatactgtttgtgatatgtaaagaaactcctcgtttctctcatactattatccagccatttgtgtgtgagtgaagccttccccaggacagctttggcacatggtatcatgtttcataatagtttcgtgtttggaaagagttgctggtaaggctgttatttaataggaggagcaaagggtttttgttttattaaatacttataaatgatcatttatcccagacatttaaaattcacacacacacaacaaataaagcaaagacaaaagaatacatttaccaaatgtaaatctgtagcataaattttttttaatttttattttaaagatggggtctcattctgtcacccaggcaggtgtgcaatggagagatcatggctcactgcagccttgatctcctaggcacaagcgatcctcccgcctctgcctccagagtagctgggactacaggtgcatatcgccagggccaggtaatgtttttgggagagacggggtctcgctgtgttgcccaggctggtctcgaactcctggactcaggtgattctcccacctcggcctctcgaagtgctgtgattacaggcgtgagccactgtgcctggaacaaattgttaagtacaatgcttttcattgtagaaaacatctcggaaacttttgaaataggctgatgttcagtgggggaggaaggactcagtcgtatagttgtcactaattttttgacttgattgacatgactcgtaaatcatagacaatagagatttggttgcttggctgagtagagtgcgtgaaaaatacacacgtacttttttttttttttttttgagatggagtttggctcttgtcacccaggctggagtgcaatggcgccatcatggctcactgcaacctccgcctccccgttcaagcgattctcctgcctcagtctccccagtagctgagattacaggcgcccgccaccacgcccagctaatttttgtatttttagtagagacagggtttcaccatgttggccaggctggtctccaactcctgacaggtggtccgcccgcctcggcctcccaaagtgctgggattacaggcgtgagccaccgcacccggccatatttttgttattaattttcaaaggctttggtgtgggaccacatttcaacatggaaggccttaaacatgttccacactacttcctgagaattagacaagatttttaacaatattgttacctagttgggacacatttgtactgacccatgggatgaaaaaaagctgagtgctagcctagtgaaaatctacttacccgaaagaaatccctcttagtctgggtgcagtggctcacaccagtgctttgggaggcccagacgggcggatcatgaggtcagtagtttgagaccagcctggccaacatggtgaaaccccgtctctactaaaaatacaaaaaattagccaggtgtggtggcaggcgcctgtaatcccaggtactctggaggctgaggcaggagaattgcttgaacccgagaggcagaggttgcagtgagccgagaccgtgccactgcacttcagcctgggcaacagagcgagactccgtctcaaaaaaaagaaaaggaaaaaagagtccctcttaattatcagcatgtgtataggcctacagatacttcaggaatacctttaccattatcatcaacttgtatctacatagcatgtgaagattcaacaatttagttttttgggcgtcctcaagagtacgcacctataaccatatggcccaattgttaatctcctatacagtccattctgggaatgtttgggcttactgtgccatttttccgttcactgccttcccctctgcaatatacctttaacccttgctaggtcctgggtttggagagccagagaaccaactttggccctaaagaagctgtgtaggtagcaatatctgcctacgaagggccttgcaaccatttcctcttggaaccttggtttcctctttctgagtagtcactttgagtaccctttattaagttagaatgtaaaaacagtttctcactgatatatctgcagtgcctgagagagggcctggcacagagtaagtactcaataaatatttgaatggggccgggcgtggtgagacctgtctctacaagaatgaacaaaattagctgggcgtgttagcacatgcctgtagacttgggaggctgaggtgggaggattgcatgagtctgggaggtcgaggctgtagtgagccatgatcgcaccactgcactccagcctaggggacagagcaagatcctgtctcaaaagaaaaaaatgtatatatttgaatggataaagagatggctttgagtttctgagatatatatggtgctgtttatctaaagtaaacaagttttctgtaaatattttaaggctttgcaggccagctgtagtctctgtcacacattcttatttgtgcatgtttttcccaaccatgtaaaaatgtaaagtgcattcttagctactggggcaggttgaatttggcccatgggctagagtttgccaacccctaacttaaacctttgtactaactttatgaccactactggatttttgttgttgtttgttttagttctggtgcctgctttgtttttttttttttttttaatcctcttgctgatgtttcttggtgcagttactgtgccatttgtattggtgcttttaatgtaatgcaaactggtaataatatctaaacttgctggggttgtacataaaattattgaaaagattgaaaagatgctgagcattgactctgtggcattcattatgcccttttgtgattgctggattttagccatctttaggacatttgagctttaggagaagccaaattctgtataaatgacttgaagtgctaatagcacaggttttgaaacctctgcctgggtttgagtctcagctctgccttttactacctgtgtgatcctgagcaagttacttagtatccctgtcctctagtttcctcctctgtagtgtggggataataacatagacataacctgagagttagagtgtagagaaggctccctggcagatagtgctgtagaagtactggccattgccattactcaggtgcttgtgtttgctgaacctcatagtaagggctcggagagcactaagaggaggtgagaaatgctgctagattgacagcttgtccccagatagcccattcccgagagcaccttaggtttatacctgatttgtgttgtagttagtagtgtctctggtaatttgaactagtttcaggttggtcttgaaaacctggggaggttgggggtaaatgatttggtagcagttctcttttgtgattttatacattatctttgtagaactgcagtttgctaattctctgagcccaacacaatgaagtctgggcctaaaatcatagaatttcttttatttttttttttgtttttaatttatttattccctccctccctcctttcttcctttcttccttttctttctttctttccttccttccttccttctttcttttctttctttcttttctttctttggagtctcactctgtcaccaggctggagtgcagtggcacgaactttcttcagagtctcactttgtcaccaggctggagtgcagtggcgcgaactcagctcactgcaacctccgtctcctgagttcaagagattctcctgcctcagcctcccgagtagctgggactataggcatgtgccaccatgcccagctaattttcttatttttagtagagacgaggtttcaccatgttggccaggatggtcttgatctcttgacctcgtgatccacctgcctcagcctcccaaagtgcggggattacaggcgtgagctaccacgcccagcctattttttattttttgaggcagagtctcactctgtcacccaggctggagtgcagtggtgcaatctcagctcactgcaacctccgcctcctgggttcaggtgattctcctgccttagcctcctgagcacctgggactacaggcgcctgccaccacacctggctaattcttatatttttagtagaggcggggtttcaccatgttggccaggctggtctcgaactcctgatctcaagtgatcaacctgccttggcctcccaaagtgctggaattacagccatgagccaccatgcccagccaaatcatgagatttcaataccgctgaactttgattatggcaaagtgaacttctgctttgattaaagcttgatgagagaggtggctggggatagtttgagataagggcaaggcaggaaaatgcataatcttacgtgggctcattgtcattgtacaattcttttggtccatgtggaatttgatccgtcctatgacttaagttatgtttatttttgtttttatttttatttattttgtgtctttttgagagacatgatgttgctctgtcacctgggccagaatacagtggcacaatcttagctccgtgtagccttgaactcctgggctcaagtgatcctcccacctcagcccctcaaacagttgagattatagtatgaaccactgtgcctagccttaagtgatttttaaatttgtactgaacagtttgtcctttccttccattaaatcatattagaagtacagaacttgatatttcctgtagcaatacagtttttctttgatgaagtttgatttcaagtacttatttttcataatttaaagctattttttatagagagaattttaatcaaatatttggatgtcactattgctatatatggtattaagtatggtgaccatagtttgtaaactccaaactgacagcaagacaggaaatttgtgttagcaaaggcttttttcttactgtttgaattttttaaaaattagatacaatacagagaggagcacacaaatcattaagagtacagctcagcgaattttcacacagtgaacatgtgtaaacagcaagtaacaaaagatttacctgcatcctataacctcccattattcccttttctaggtactgtctctccactgcattcccaccaaatataaccactatgctgaattctgacatcataaatgagttttgcctgattttgagcttttgtgactggaagtgtacagtgtatataccctttcgattctgtcctctttagtttaccattgtttgagaaatttatccatactgttccagaattaactactgttaattattgttaattaactactgttgtagttaattcatcctcattgttatctagtattcttttgtgagtaaacacaatttccattctactgtgatcccagctatccatttgggtcgtttccagtttggggtccattacaaatagtaatgctatctgtaatgctattttgtattactacaaatagtaatgctatttgtggcacaaaaatactgcttttgtgaacattcttatacatgtcttttgatgaatgtatgtttgcattgctgttgtttacattatgtacctagtaatggaattgctagatcataggagatgtatatattaagctttagtggatgcattacataattattagttattattggttataccaatttatcctctcatcagtagtatacaacagtttctgtatctctaatctccaacattttagccattttagagtttgtgtactaacacattgtggttttaatttacatttccctgatgactaataaagttgagtacctcttttgtgttctttatagccatttgactgtcttgtgaagtgcttgtttgtcttgcctatttttcttttctttctttctttttcttccttccttcctttctttctttcttctttctttccttccttcttttctttctttctgtctttctttcttgtctttcttgtctttctgtctttcttggtcttgccctgtcacccatgctggagtgcagtggtgcagtctcagcttactgtagcctcgacctttttggggctcaagttatcctcctttctcagcctcccaagaagctggactacaagcacgcaccaccatgctcagttaattttttattttttgtagaaatggggtttcaccatgttgtccaggctggtctcaaacttctgggctcaagtaatcctcctgccttggcctcccaaaatgctgggattacaggcatgagccaccgcagccagccttggctatttttcaaaaggatataagtagaacatctgtatatcccttcaatttgcatattattcagtaagagttgcactctggtagtagaaatatataaggaggagaaagaagtggaaacaaaaagtctattctcatgagaagacttgggggatagtgttctctctagctccaagctacttattccttacgaaaagttgaagataaacttatctcagactgaggctgtctcaatgttgtcttcctattccattatacacatataacccatatttttttcaccagctgaattttgctcctagaaaattgattcatcaggaaaaatatccgtcttgcaaggtggttctctttagagtctgctgtgtgacatagctcaggacaaattgtgtgatgtcagataggttgggttaaggaatagaccttattggggaaagagagaacttggagggccaaggttagcaggagaaggaaatgttctctcatctgccgtcaattcagggaggggcaaacctggtgtctgtgttcacagggagggatccatccatctgtgattctcccttcttatcaggtagcatgggaaagctacactgttgcggggaggagggtcacacgcaggctacttagtaccaggcaccctggacttggattcaggttgccagttgtgtgagaaactgcccagcacctgaaggccctgaacccatgagaagttgtacctacctcccatgaggaggaatcctgtcatcccatgggagctgagcttgggtgcagtccctcttgctggcttgtccaggagtgagctccagggttgtttgggacagttctgctcattgctttacactgtgtatacattatctgtagagttccatgaagagaacttcagcactgtaactgcaagttttaacatggaacagaatttttctcacctgtattaattcttaagatttgaagttctatcaacaagcatttagattgtgtggagatttttttatttttatttttggagacagagtcttgctctgttacccagactggagtggcagtggcatggtcttggctcactgcaggctctacttcctgggttcaagcgattctcatgcctcagtgtcctgattagctaggactacaggtacacaccaccatgctggctaatttttgtatttttagtagagacgaggtttcaccgtattggtcaggctggtctcgaactcccagcctcaagcagtccacccacctcggcctcccaaactgctgggattacaggtgtgagccaccatgcttgactgacatcatcatgttaaaagaataaatgttctagggagctgggcacagtgtcatgtttctgtagttctagctgctcgggaggctgaggcaggaagatcccttgagccctggagttcaagtccagcctgggcaacatagtgagatctctttttttaaataaataaataactgttctagggactaaaatttcctttcaccattagtaatttactgtagaatctccaagaatgaacttattttaggtactgaaaatgagggagactaaatgttttatacagtagtttttagtaaaatatgagatttgatgcatttgatagatgatgtttgtttaaaataattcttaaatttttgatcatgtaattatagtttcattaatggtagatttgtaaaataaatgttaccaaatgaaaatgcatgtacctatgttaattatccttatctaaagctgaaagttcagttcaactatgttaaaacatagtaggggcctggcagggtggctcttgcctgtaatcccagaacttagggaggccaaggtgggcagatcacgaggtcaggagatcgagaccatcctggctaacattgtgaaaccgtatcgctactaaaaatacaaaaaattagccgggcatggcggtgggcacctgtagtcgcagctacttggtaggctgaggcaggagaatggcgtgaactcaggaggcagagcttacagtgagccgagatcatgccactgcactccaggctgggtgacagagcaagactccatctcaaaaaaaaaaaaaaagttggccaggtgtggcggctcacacctgtaatcccagcacttttggaggccgaggcaggcggatcacaagatcaggagtttgagaccagcctggctaacagagtgaaaccctgtatatactaaaaatacaaaaattagccaggcatggtggtgcatgcctgtagtcccagctacttgagaggctgaggcaggagaatcacttgaacccgggaggcggaggttgtggtaagctgagattgctccactgcactccagcctggacaacagagcaagactctgtctcaaaaaaaaaaaaaattaatgattaaattatttaggggagccgggcgcagtggctcacgcctgtaatcccagcactttgggaggccaaggcgggcggatcacgaggtcaggagatcaagaccatcctggctaacacaggatgaaaccccgtctctactaaaaatacaaaaatttagccgggcgtggtggcgggtgcctgtagtaccagctactcgggaggctgaggcaggagaatggcatgaacccgggtggcggagcttgcagtgagccaagatagcgccactgcactccggcctgggtgaaagagtgagactccgtctcaaaaaaaaaaaaaaattatttaggggaagatactatacaattctgtttaacaagtcacattttaattttttcttttggaaatattagcaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacaggtactgattttaaactcactaagtcacatttcttttttttttttttttttgagacggagtctcgccctgttgcccatgctggagtgcaatggcgcgatctcggctcactgcaacctctgcctcccgggttcaagcgattctcctgcctcagcctcccaagtagctgggattacaggcacacggcactatgcccggctaattttttgtatctttgttagagatggggtttcaccatgttggtcaggttggtctcaaactcctgaccttatgatccacctgtcttggcctcccaaagtgctgggattataggtgtgagccaccacacccggcttacatttcttttaaaaatgtggataccatttagaaaaggatgggccattcttcctatagggatctgactggtgaattataactgtgctgttaactttggaaatgggaatgcacaagatattgttttaaatatgcacgctaatgacagtttgtatccttctttccccacccccacccttgcttcaactacctgtcaaaattaacagcagccttctggaaatatggatgacagtggttttttctctattcaggtaagtagtcacaagcatgtactatgtgttgcttacatcccaggcaccgtttcacagcctttcaatagtcactgtaacaaggcgaccttcggaagttcttctgtctacagagtatagattatactctagagtactagattttttttttcttgagacagagtctcgttctgtcacctaggctggagtgcagtggcgtgatcttggctcactgtagcctctgcctcccgggttcaagcgatcctcctgcctcagcctcccaagtagctgggattacaggcacccgccaccacaccagttaatatttgtatttttagtagagatagtggggtttcaccgtgttggccagtctggtctccaactcctgacctcagcctcccaaagtgctgggattacaggtgtgagccactgcacctggccaactagagtactagatttttatatagataaacatgaaaggattgtagaatcttcatattagagtggggcatttaaaaattccttcttgagaaagattaatttgcatctggatgctaataataaccttaattctggccgggcgcggtggctcacacctgtaatcccagcactttggggaggccgaggtgggcggatcacgaggtcaggagattgagaccatcctggctaacatggtgaaaccccgtctctactaaaaatacaaaaattagctggacgtggtgacacgtgcctgtaatcccagctactcgggaggctgaggcaggagaatcgcttgaaccagggagtcgtaggttgcagtgagccaagatcgcgccactgcactctagcctggtgacagagcgagactccatctcaaagaaaaaaagaaatccttaattctaataagtcacaatgtctcaaacttaccatctgttgggtaaatttgagaaaatgcaataccttgctaccatccttttaaatcagcctaccagactggatttccttattatggtttgtggcttttgattttttttttttaatgtatagctctctttgaattctttggtggttatatatatatgtactcgcaagattcttttatctgtgggtctttcattctttttctaacactgtgagttgtatccagagtactttcggaacctctcctgagcgacctatctctgcagatatctttgtttatgtttcccttgtactgccctcctggactcttcctcatccaccagcatttccatctagtgctttaccgtgccactgctaacaggtaatggctactgcagggctgaaatcagaggccagagtaggcccagcacttggcgtttcctatttgtgccttgctgctcttggtgcctgttcatgtgtgcccactaccttgcactcaatttctgtctttgctggtacctggctcacttgcttctttgttggctaccttggagggcagatagtgaattttcagaaatttccctttttttgtcagacagattgaaataaacaggtttgcattttgttttttctacaagcggcaagcccatgaccctagaagtctgacatctatggaaccttcagtttaaatgcccagggagaacttattttggtagatatgatttctgacattgcaggtagcaagttgaatataatttttctaaagtagcacccacagcagccaaattatcagatgtatatagtagactagttttaagaaaagcacttatgggtagaatatacatctggatttttgaggcagttttatttaggaattgtgtggttttctggaacatctcagagacctggtatgaaaagcactcttctaatatatatgtgtttttttttatggatttagtgatatatctatacacacacactttttaaaacctatagccggctgggcgtggtggctcatgcctgtaatcccagtactttgggaggcccaggcgggtggatcacaaggtcaggagattgagaccagcctggccaacaaggtgaaaccctgtctctactaaaaatacaaaaatagctgggtgtggtggcgtgtgcttgtaatcccagctactcgggagcctcaggaggagaatcgcttgaacctgggaggcggaggttgcagcgagccgagatcgtgccactatactccagcctgggcgacagagcaagactctgtcacaaaaaaaaaaaaaaaaacctatagccttctagagaaatttatatatgaagtacacaactaacatagctacacttcctaaatttggaatggagtggtttagcttatgaaaagttgctatttttcttaacaggttataagcaatgccttgaaagtttggggtttagaactaatcctgttcaacagtccagagtatcagaggctcaggatcgatcctatgtaagattctgttttgcatttcatacatttcttttcccaaatttgatttttaaagttgtaatttcttaaagaagagaaatacattttgaatacttttgttttgatgttccctgtttcattcactcagactttcctatttcacctttgtgatgtccatgagcatctgccctgtagccttcctggcaccccagtgtctgtggcagcacagagctgaccccataagtggtgcatgaggccatcttgtggcacagcatcactaagctgctgcagagacgttcatatggttgtgtgatcttttaaaaacatcagtgacacttaactataaatataatcttaaattatcacaaattttatataatatttgccagtagacaacataaatatgaattcaatatttcaagttaatattgtctgttttcttttttagaaatgaaagatcatttatatgcaattataaggaacactggtttacagttagaaaattaggaaaacaggtaacatttcttacccttccttgtctttttttcttatattgtaccccatttaaaactaaaatgtgggccaggtgtggtggctcatgccaacagtttgggaggctgaggtggggggatcacttgaagccaggagtttgagaccagcctgggcaacaaagggaggtcctgtctcttaaaaaaaaaataaaaataaaaataaaaataaataaaaaaaaaaacaaagagccaggcatggtggctcacatctgtaattccagcttacttggaaggctgagtcagaaggatcacttgagctcaggagtttgaggctgcagtgaactatgattttgtcactgtaccccagcctgggtgacagagtaagactgttctataaaacataaaaataaaaaaaatatatttaaaaattaaaaaaaaaaaaggattgctgactttaaaattaggaaactgaccagtaatgtgtgtgtgtgtagcatggtttatccttcttgatagatagaaattgtcattttaaaagataatatcagttttccttataaatttatttgtgacaagtatatgcaatttaactatatcataagaaaaattctatattaaagataatacaaatgtggttacttttaagtgggtttttatgtgatgactatgttctgtcagttaattattacatttatagatttgtatttagcatagtgctgtcacaaagcctgaaatagtgtcaagcatgaataaagcattcaattatgtttgctttagtgtaagattattcattatgattccaaaagccatgtaatacgtacgtctacagaaaatcacttctattttttaaataaaacatgaaatatgtcttgagcaagctattttaagaaacaatcatttaacgtccttgttattagaattttgaatctttgaaagagggttattgaaaaccagctaggacagtaaaaaagaataaactagtgatacatgcagcaatatggatgaatctcaaaataattatgctgaaagaataacccacaaacaaaatactacctgctgtatggtatcatttattaaaagtctagaaaagtgcagattcatctgtagtgatggaaagcagattgaccagcggttgcctggggacgagaaggctatggaggagtgagaggggagggttacagagaggcacgggaaacatggcaatgaggaatgtgttcactatcttggttgtagtaatggtttcatgggagtacagtatacaaatgtgaaaacatttcagaggccagatgcagtggctcatgcctgtaatcccagcacttttggaggccaaggcaggaggattgcttgagctcaaggagttcaggaccagcctgggcaatggcacaagaccccatctctaaaaaaaaaatgaaagaaaaaaaaattggctaggcgtggtgatgcatggccgtagtcccaggtgctagggaggctgaggagggagcacagaggtcaagcctgcagtgaatcatgatcgtgctactgcactccagcttgggtgacagaaggagatcctgtctcaaaaaaaaagtttcaaattatacactttaaatatgtgcagtttattatatgtcacttataccccaataaatctgttttttttaaaatgtaaatacaagccaaaaaaggtataagtcaagaaaatatattgaattaaatctgtaagagataattcaaaaacaaaaaccctattgttatcttttaagtcacccaaatcaaatttgggaaaagtcacctacttagcttcatcctaagttggttctttctttctttctttccttcttttgagacggattcttgctctatcgcccaggctggattgcagtggcgggatcttggctccctgcaacctccgccacctgggttcaagcaattctcttgtctcagcctcccaaatagctgtgtctacagccacgcaccaccacacccagctaatttttgtatttttagtagagacggggtttcgccatgttggtcaggctggtcttgaactcctgacctcaggtgatccgtccgtctctgcctctcaaagtgctggggttacaggcgtgagccaccatgccgagccctaagttggttctttcttaaagttcttcctgaggagccaagagcaagttaaggagatgtaacctagaagcttacagtggaggctagctgggtgcagtggttcacgcctgtaatcccagcactttaggaggctgaggcagggagatcactgaggccaggagcttgagagcagcttggcccaacacagtgacaccttgtctctacaaaaaaaaaaaaaaaaaaaggcagcttacagcagtagaggctgatgcgagtgggaatcacctctaggtaaaaaccagtgtagcgtactgctgagattatttaacctctgggttttatttatgtgtttttaaaaattatgatccagtattttttacttttttttgtataaagtaagcactgaatttttaaggttgtattaatttgcaaataaatgtctatcttattattttgagagatttaaaaaattttagttcttcaaaattgcattttcacattttgaattacgttatctttgacaaatacagaagatgtcaaattttggtttattttctttggttctaatttatatttttgtttaaaactatatttttcactatagactctttctgtctctcgaggtccctgtataatgaaaaagaaggctggaaaaagtattaacattgtcaaaatccaggaaaagtagttggtcatgatattgatcgttaactttagaaactttttgtatcttgtgggttaaattaggattactatgtggtagtgataaatgatgttaattagggccgagtgcagtggctaacacctgtaattccagcatgtagggaggctgaggtgggaggatgtcttgaatccaggagtttgagaccagcctgtacaacatagtgtaagaccccttctccacacaaaaaaattagaaaatttgtcaagcatcttggtgcacacctgtagtcccagctgcttgggaggatgaagcgagagaatcacttaagcccaggtgttcgaggctgcagtgagctatgattgcaccactgcactccagactagatgaccatctcttttaaaaaaatgtgtttatatgttatatgtgatagtgctttttaaaaacatttttaaattatagagacagggtctcactatgttacagcccaggctggtctcaaattcctgggctcaagcaatcctcccaccttagctaacctcccaaagtgctcggattataggcatgagctgcatgcccagctaatttagtgatttttaaaaactgagctggtaattataaattctcttcctggaacttctgactttctcacaattggaatcttttgacaaaaattatcagtaatgggaaaactttgtgtagttgtcatttttcctcccatcagtgtgatagatatgattggagttatgttggactgatattttgaaaaaagatttaattatagctattaataaagacatttaaactactgactatgcatttttattcttttgggagggtttaatgtttatagtttaaagcaaactgttgtttttaaaaaagtatctaacagggccgggcgcggtggctcacacctgtaatcccagcactttgggaggcctaggcgggcggatcacaaggtcaagagatcaagaccatcctggctaacatggtgaaaccctgtctctactaaaaatacaaaaaaatagctgggtgtggcggcgtgcgcctgtagtcccagctactcgggaggctgaggcaggaggatggcatgaacccgggaggcggagcttgcagtgagccgagatcgcgccactgcactccagcctgggcgacagagcaatactctgtctaaaaaaaaaaaaaaaaaaaaaaaaagagtatttagcagaggccaggtgcagtggctcatgtttgtaatcccagaactttgggaggctgaggcgggcggatcatttgaggtcaggagtttgagaccagcctggccaatgtggcaaatgtgctgtctctaactaaaaatacaaaaattagctgggtgtggtggtgcagacctgtagtcccagctacttgggaggctgaggcaggagaatcacttgaacctgggaggcagaggttgcagtgatccgagatcatgccactgcactccagcctgggttacagagtgagactcttctcaaaaaaaaaaaaaagtatttaatagtgataaatctgcagtattctcttgtagtttttaagatcatattattcagtcaaagaaaagagctcaacttgaaatatttccagagtttaaacaatcttactaagctttgatgggttgtatctattcttaacatgtgaaacttccttattacctataatatacactaacttaaatattgacaatttttttccagtggtttaacttgaattctctcttgacgggtccagaattaatatcagatacatatcttgcacttttcttggctcaattacaacaggaaggtaagtaacggctgaacattttgtaatgttacctttcgaagtagttaaataaccaggcacattagatgacagtgtgataaaactgtttttctggcagtggcagtgaaacaatctttagttttgacgtggtgataggctgtgatttgggtgacgctgttcagttagagttctcactgacacctggcccttcctcttctgaggatgctgctttctttgcagcccttctaagtaatggctttttcttttatacatcacatatcacacggctgagaggagggatagatgtttttcttctttgcctcttctaggccactgttcttccttataaactccagtttctttgaaatacatgcccctaacggctgggcacggtggctcacgcctgtaatcccagcactttgggaggctgaggcaggcggatcacgatgtcaggagatcgagaccatcctggctaacacggtgaaatcctgtctctactaaaaataacaaaaaattagccggggtgtggtggcggacgcctgtagtccgagctactcgggaggctgaggcaggagaatggcgtgaacccaggaggcggagcttgcagtgagctgagatcgcgccactgccctccagcctgggcgacagagcgagactccgtctcaaaaaaaaaaagaaaagaaaaaaaaaagaaatacatgcccctagattaaactatcccttgtccttttgcactcatccacaagtctcttttcatcagtgattttaggatctgactcgttgtctttttctctacttcaactacttttatcattcttaattatttctgtatcgtcaatcaatccagtacctgcctcttagtttcaaaatcacttactcttgcttagctattaccagtaatcataaccactgtcaaatctcaattgcaagcatattactctttaactaccacctcctatctttaaaccatgttttgtctgtttttttattccagccattctttaaaccctactgtggggcccaagcatttcctttatacgcattcttcctttcttctactgcttattttctgtaatccgtcatcataatcactccattgcattcttcaacgtgtttcccctctctccctccatcatacttgaatgacaaaaatctcaaccctggttaaaccacatcttggccttgtccattcctgtaccagagtagctggacgtggctaaaaaataacataaaacatgatgattggttttacttttttcttaaatgatctatccatccattcacccatccatctatcaaagtgactaggcctatttctgaagcccaggctggagtgcagcagcataatcacagctcattgcagctccaaactcctgggctcaagtgattctcttgccttagcctgttgagtagctgggactacaggcttgtgctaccacacctagctaaggttttactttaaatttattataatcacaaaattcagatgagcctttagtgctgtctgatatttctactatgttttcttagtgatgtaccaccctccaaggtgtttataaaaaattatgtaccactctccaagaagtttataaaaaataatgtgccaccctccaaggtgactaatttcacagcttatgtctttaaacctttaagcactttcctctcccttacacaccttccttgtggctttccgttacattctgctgagaacatagaagcaattaaaattatgttctttctaccagcaaatttatcaatttgcttatatcttcacctgtgctttgagcctatttaaatagatgaatggtcccctacctctaaccaaaaccagtccctcacttgtgggctggatcccagctcttctcacctactcaagatgttcctgctttcatctctccactctcttatataatcagttccccccccctttttttgtaatattcctataagcagtaaaataagctttttatttccattgattaaaaataaaaatcctctcttaattccatgaaactccagctgcctccccatttttattttttccttaggattgtctctagtgtgccttctccttttcttgaactctgcctcctgggttcaagcgattctcctgcctcaacctcccgagtagctgggattacaggcgtgcaccaccatgaccggctaattttttttttttttttttgagatggagtttccctcttgttgctccggctggagtgcaatggcgtgatctcggctcaccgtaacttctgcctcctgggttcaagcgattttcttgcctcagcctcccgagtagctggatttacaggcatgtgccaccatgcctggctaattttgtattttagtagagatggaaggggtttctccatgtttgttaggctggtctccaactcctgacctcaggtgagccgcccacctcggccccctaaagtgctgggattacaggcatgagccactgcgcctggccccggctaaattttttttttttttttttgtatttttagtagagacagggtttcaccatattggccaggttggtctcgaattcctggcctcgagtgatccacctgcctcagcctcccaaagtgctgggattacaggcgtgagtcaccttgcctagccatcttttagtaatggtatttggagatcacaatttgagtgctggcatgcttattgctgctgggtttgttatgtagttattgtgaattcacatttaggaatatagggtttttaattctttgattttagatacttgtatcttttttcttttatatttaaaaccttggttcctgatgatatcccttcttagaaaccctgtctacctttggccttcagcccaccatgctgtggttttcctaacttgctgcctgcacttttcagattcctttcatggatcttaaatatcatctgtaaataagatctatgtgtcaataattaccaaacttttatctttagtcttgacatctaccctgaacacctagctttgactaactcctagctttggcatctccacttggaaatccaaaaagtgtttcaaactgaacatgtctatgaaagacttatttttttctctctatccatgctatccatcaggttttccatttccataagggtgactcttgtactctggttcctatatattataccgacagagcagcccagagtgcttcttaaccagtgtaaggcctgttatgtcccaccctcactctttgtccttcagtggcttcccagcacacttagaataaaatctgaagtcttaggccgggcttggtggctcatgcctgcaatcccagcactttgggaggatgagggggcagatcacttgaggtcaggagttgatgagaccagcctggccaacatggtgaaaccctgtctctaccaaaaaatacaaaaattaactgggtgtggtgttgtgcacctgtagtcccagctactcgggaggctgagataggagaatcacttgaacccgggaggcagaggttacagcgagccaagatcataccactgcactccagcctgggtgacagaacgagactctcaaaaaaaaattaaaaaaaaaaaatatgtgaagtcttgaataaaacccaagatctttaccatggcccctgaacagggcagagtatccattcttcagacactcttcatagaataccatggtgagctggcatatttattatacaatacagaaacaattttactggcagaaaacacattaaaccgtctaaactctgaatacagttgtcctcataaaaaatgttcaacatactattttgaggttttccattaatagttcttataatctttgtcccattatgtgttaatccaacaaaggatatccaataacaaacaccaaagtttaagaaaaatgtgctaggcgcggtggctcacacctgtaatcccagcactttgggaggccgaggtgggcagatcacctgaggtcaggagttcgagaccagcccagccaacatggtgaaaccctgcctctcctaaaaatacaaacattaactgggtgtggtggtgggtgcctgtaatcccagctactcaggaggctgaggcaggagaatcgcttgaacctcctgggaggcagaggttgcagtgagctaatattgcaccactgcactccagcctgggtgacagagtgagactccatctcaaattaaaaaaaaaaaaaaattaatgatagagaaacttaaatcagttagattgttttaggtatagcccatccttggtttttgtgtgtagcatctagcttggggaaaccctggatttctggaatcatatttagacacagtcacactagactaatgtaattcttttgggatgcaaaccacacgtttgacaccttaaatagcttttaggtatttggcttcccagcccctatttttagttacaaggggtgtacatgtgtgggtcagggtgggggtagctctttccgcagatgattagttttagccatgttactagttattgcacacattatctgtgtcctcacagcagccctgtgagtaagtgtattagggttctctagagggacagaactaataaggtagatgtatatatgaagggtaatgtattaaggagtatcgactcgtatgatcacaaggtgaagtcccacaataggctctctgcaggctgaggaaccaggaagccagtccaagtcccaaaacctcaaaagtagggaagctgacagtgcagccttcagtctgtggcaaaaggcctgagagcccctggcaaaccactggtgtaagttcaagagtccaaaagatgaagaacttggagtctgatgtttgagggcaggaagcatccagcatgggagaaagatgaaggctcagcaagtctagtacttccacactcttatttctgcctgctttattctagctgagctggcagctgattagatggtgaccacccagtttgagggtgggtctacctctcccagttcactggcttaaatgttaatctcctttggcaacaccctcgcagacacacccagaaacaataatttgtagccttcaatccaatcaagttgataatattaaccatcacaggaaggtactagtatcatatgtttaacagtagaaaccaagacaaatgcagctaggaagtgggagaactgggatcagatgcaggcagtctgattctaaatcagttgctgttacccactctgacaacagtaagtgagtagcctgctcagtcaagtactatattagtagggccctttacagacatatttatttctcacagtcactcaatgagacggctcttccagtcttacaatggagaaagtgaggctcagagactttaagtaacttaccttagacgactttactagtaagtataagaatcattatttggactaaagtctttctgaatcctcagcttgtatttttttccagtgttctgtgctgcctttttatctactagtgttttacatcaattttgaatctctttactaactggttaggttgatttttgccttttttttttaggttattctatatttgtcgttaagggtgatctgccagattgcgaagctgaccaactcctgcagatgattagggtccaacagatgcatcgaccaaaacttattggagaagaattagcacaactaaaagagcaaaggtaaaaatgaggcctgcagtatggaatatatggtagtatttcattatgagaattaaattttcatgcttagattgaatatgtggtccttgtgttgttggcgactctattttggaccttatattttagtgaagtttattagtttaaacttgaatcaactctttgaaatacttaaatatattaacttagttagctggtatggtatattcctagcacttcgggaggctgaggcaggctgattgcttcaacccaggagttcgagaccagcctgggcaacatggcaaaacctcatctctacaaatagtacaaaaattagccagatgtggtggtgtatgcctatagtcccagctacttgggaggcagaggaagaaggatcacctgaaactggggaggtagagactacagtgagccataatcacactaccgcactccagcctggtcgagagagtcagaccctgtctcaaaaaaaaaaaaaaaaagaaacggaaaaaaaaaacttagttggattcaaattgcaacacaatcattatattactagagcttatttgccagaaaacattttaagttttgacttacttaaagcctttacattacaaatgcctttatgttatgtctaaaatagaagattggttgcagttattaccagtgcttttgttctttagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtaaagacattctgatgtgtgttgtattcattgctgaagaattgattccaattattcttagatttcatggaagttaatgtactcttagaggtgttttgacaattactgcagaagcaatagctatatagtgggctttccctttagatttcttataatggaaatcactttttacaacctatattttattaggagtagttatatttttactcctggttattttatttggtttcaacactgtactaacacaatagtaaattgtggttttaatctttgtgggtatcagttgacccttatccaaatcagctgttacataaatatgtgccattagacactatggaagggcctggacagggaatataaactgattttacaaaaacccaacatttattggctatgcaacttaaaccgtaagcccactttggtgggcccagttttttagtgatataaactatcaatagagaaaagcgaaaacatatcccctagacaatctaggcaaagaaaaatgttaagacatagctcaaagtagcttaattaaaagtttgaagtgggttttttgttttatttttttctaactcatatgtatttgcttctactttctaatgaaattatttatcagttgatttccttagatatctaaataaaattgaaatttcattaatgggaagattatttttatcctgaacttttcttgcctctatgcatgcctctgagtactccatatggtgtgcaatcccatttttgattaatagagtcctgctggattagcagggacagaaatcagctttagatttctttcttttttttttttctttctttttttttttttttttttttgagtcagagtctcactgtcgcccagcctggagtgcagtgatcttggctcactgcaacccctgcctccgaggttcaagcgattctcctgcctcagcctcctgagtagctgggactacaggcgcctaccaccacgcccagctaattttttgtacttttagtagagatagggttttgcccttttggccaggctggtcttgaactcctgacctcaggtgatccacctgccttggcctcccaaagtgctgggattacatgtgtgagccaccacgcccagccagaagagtagaatattcttaaagagaaaacgttttaaaggcttactcaaatgagtataaacaaacatattgttgcttgaattggtaaatacagtgattggtttttgttgtgttgtgttttgttttcaggtagttccagaaacatatctcaagatatgacacagacatcaggtacaaatcttacttcagaagagcttcggaagagacgagaagcctactttgaaaagtaaagtagttggtacaagttaaagtagcatgtttaatatttgctttggctattttgtctatttgtaaatggttactgcctgaatcctgtgaatatttgaatgtattttttaaaaatttacagcaaataggacgggcacggtggcttacgcctgtgatgctagcagtttgggaggccaaggcgggcagattgcctgaggtcaggagttcgagaccagcctgggcaacacagtgaaaccccatctctactaaaaatacaaaagaatcagctgggcatggaagcgtgcgcctgtagtcccagctgcttgggaggctgagccaggagaattgcttgaacccgggacgtggaggttgcagtgagccgagatcgcaccactgccctccagactgggtgacagagtgagactccgtctccaaaaatatatgtatatatatataaataaaaataaaaatttacggcaaataacatgaaacaaaaaaaccttgccccaatactggataaattttttaaactgagtgaaggaaaccttataaaatttcatttattaaaagaaaaatgaaattaggacaagacaagaagaatgccaattgatcctttggatgtacttcttgcttacctgattaaccctgcaaaattcctctaccaatcagtacgaaaaacagctttggaggtatgggagcgcattcccaaatagacgtggtagttcatttagctgctcatggccgcttcaggcagtcctgtaagcctgttagcatcaggggaatggatgcaaaccataaatctggatcaactcctaaaaccttaccttgtgcccagccttgtaagtgcttgctaaataggaattccaccatatgaaaatacattcttttcaagtaactatcattcagacttttgtcccccactttttttttttaaagaaaaataaaaggctgggcacggtggcttacgtctgtaatcccaccattttaggaggccaaggcaggtggatcacctgaggtcaggaattcaagaccagcctgaccaacatggtgaaacctcatctctactaaaaatacaaaaattagccgggcatggtggtgggtgcctgtaatcccagctacttgggaggctcagacaggagaatcgcttgaatctgggaggcagaagttgcagtgagctgagataacgccattgcactccagcctgggggacaagagcgagacttcgtctcaaaaaaaaagagaaagaaaacttcatgttaaagattacaagataaataatcagacccactgatcctaggtcagaaaacagagtcatagctcaatctgacttactatttgctgtatttcatccattctgagatgcacatagtttcacatttcaatgtctctgaaattgagaagcatcttacagtcataattgacagtatattagcagcacctataaatattggctcattttacatttgatggtataatgaagaaaatatttacctttttttctgttttgtttttaagtcacaactcagaagtagatgaaggaaaattctgatcagctgacatcctcttaatgtgagatatttctagtctttattcagtatagattaatggctaattatatgttaaatttcaaagtagtgcttattagtgctttttacttttaagtttcaaaattaacttttttattataataaactccaaatttatacaaaagtagaaaaactagcatactcctgtttatgacccagattcaacaaatactagcacacggccaatcttgcttttttttttttttttttttgagatggagtcttgctctgttgcccaggctggagtgcaatggcacaatttctgctcactgcaacctctgcctcctgagttcaagcgattctcccacttcagcctcccaagtagctgggattacaggtacacaccaccatgcctggctaattcttgtatttttagtagacacgggatttcaccatgtcgtccaggctggccttaaactcctgacctcaagtgatccacctgcctcggcctcccagagtgctgggattacaggcatgagccactgagcccggcccaatctcgttttataatactcccatctcccattctttccactgtcccacctgcaagtttggattattttgtaacaaatctcaatcatcatattattctataaccattttaatatgtgtctctaaaatatattagctttatttttaacatagttaaatgctattgtcataaaataataatcataataattaattgtaattctatatcatcaattatctagttaatgtaaaaaataaatctaaggccaggcgcggtggctcacacctgtaatcccagcactttgggaggctgaggtgggcagatcacctgagatcaggagttcaagaccagcctgaccaacatggagaaaccccatctctactaaaaatacaaaaaattagccaggcgtggtggcgcatgcttgtaatcccagctacttgagaggctgaggcaggagaatcacttgaacccgggaggcgaggttgcggtgagccgagatcgtgccattgcactctagcctgggcaaaaagagtgaaactccatctcaaataaataaataaataaataataaaaaataacttaaatctacttaattagaaaaactaacattctaaaaattttattttaagaaatatcaaaattggctgggcacggtggctcacgcctctaatccctgcactttggaaggctgaggtgggcggatcacctgaggtcaggagggtcaggagtacaagaccagcctggccaacatggcgaaaccctgtctccactaaaaatacaaaaattagccaggcatgatgatgggcacctgtaatcccagctactcaggaggctgagacagaagaatcgcttgaacccaggaggtagaggttgcagtgagctgagatcaccccactgcactccagcctgggtgacagagtgaaactccgcctcaaaaaaaaaaaaaagagaaaagaaatatagaaattaaagcatacatggccaggcgtagtggctcatgtctgtaatcccagcactttgggaggctgaggcaggcagatcacttgaggccatgagttcaagaccaacctggccaacatggcgaaagcctgtctctactaaaaatacaaaaaaattagttgggcatggtggtgcacacctgtaatcacagctactttggaggctgaggcaggagaatcgtttgaacccagaggtggaggttgcagtgagccgagattgtgccactgcactctatcctgggtgacagagcgagatactgtctcaaaaagaaaaaaaaaaggctgggcgcggtagttcatgcctgcaatcccagcactttgggaggccgaggcaggcagattacgaagtcaggagatggagaccatcctggctaatacagtgaaaccccgtctctactaaaaaatacacaaaaattagctgggtgtggtggcaggcacctgtagtcccagctactctggaggctgaggcaggagaatggcatgaacccgggaggtggagcttgcagtgagcagagatcacaccactgcactccagtctgggcgacagagcgaggctctgtctcaaaaaaaaaaaagaaagcatactctcacctccttcagtgactgatgttagtattttggcacattctttttctgtgacatatacacacttaccttgtaagtgttgtactcatttcctatgacagtaaatagtctttgtaacaggctgcatgatatttcataaaatgaatggatgtggcataatttatatgtgagccttttgaattctgctattataattaatattgcaatgaacaattcttatattgcctctacacctcaaatgtcttatcatttcttctagtttttctgaggatgtcagattattgggttaaaggatatgaacatttttaaggccttggaacagatttctaaattgctttccagaataattcccatgtgatactttcaccatgtttatttcagacttttttttttttttttttttgagacgaaatctcactctgtcacccaggctggagtgtagtggcatgatctcggctcactgcaacctccgcctcctgagtttaagcgattattctgcctcagcctcccaagtagctgcggttacaggcaagtgcctccatgcctggctaatttttgtgtcttttgtagacatggggtttcaccatgttgcccaggctggtttcgaactcctgagctcaggcaatctgcctacctcggcctcccaaagttctgggattacaggcgtgcaccaccgcgcccagccatcagagtcttttttgtcaaaataaaatggtctaaagacatacatcatagagaaactataatacaaaatttacaggtatatctaagaaaagaaaagtatatttaaagcataaaaataaactgctcttttacttaaaattttttaaaaactggattaaaaatatgaaacttccaacaaattgagcttttttttttttttttttcttttttgagacgaggtctcgcttttgtcacccagtctggagtgcagtggcgcgatctcggctcactgcaacctccacctccctggttcaagcaattcccctgcctcagcctcccaagtagctgggattacaggcgcatgccaccacgtcgggctaatttttttgtatttttagtagagagggggtttcaccatgttggccagactggtctcgaactcctgatctcaggcaatctgccagcctgggtctcccaacatgctgggattacaggcatgagccactgcactcggcctgaactttttatagtagtaacgataattcagtaatgtccaataatgactaagtaagttataacaagtacaatgtcagcaataactagtgctttttagtaaacagggtcaggcaaccttgtacccttttaaaaatgttcgaatatcgatatacctccttcctacttggtggaggattgattgaggaggaaagtgtgcagtgatggttaccagcttcagcctcttggcttgactttgcaaatactggtgagaatttggaaagagcttgagaatatcttacatagtcacatgttgctgagaagagttaagaactaacttcttgatgttcatttttaacaatggcttgcattcaaaaccttgtagagctcattagtaggagctaagaagctaatatttgcctttcactaaaattcctgattacttagcctaggtagttcgttgtctctctaggttctgtctttgggagcttgggtctaaggttatcaagctaactctttcttccctctcacccttcccaaattgaccctggtgctgatttgttattcatacgattttctagtttttcttttccctttttgagtatttgaagcttcatactgaatatagtaatcatagtattcatgcataaagaaaatcataaagtaattgcataaatgcataaagtaatcatagttttcatgcattaaaaaaactagttttggctgggcgctatggctcacgcttgtaatcccagcactttcggaggccaaggcaggcgaatcatctgaggtcaggagttcgagactagcctggccaacatggcgaaacctcttctctactaaaaatacaaaaaaattagccgagtatggtggcgggcgcctgtaatcctagctatttggcaggctgaggcaggagaatcacttgaacctgggaggcagaggttgcagtgagccgaggttgtgccattgcactacagcctaggcgacaagagcaagactccatctcaaaaaaaaaaaaaaaaaaaaaaaaactccctattacagattcataatttatgagtcattaaataatattttcaagccatgacattttttccagcagtagtctctaaatctgttttaccatcataaaaccccaagcaaaactctactacatcagctgtgtcactgtaaaacctgccttaactcacagaagcatgaaattaagcaatgtgtgtgaaactattttataaactgtaaagtattccatacatacatgttggcagttattaatgtcttctctaggtgtggctttgaaatggatgcagatgctttctgttacaaaaaacataagttgcaaatgttctataacaaggagagacacaaatatcttcatggacatggattgctatgagtgtttgattgcctaatacttgagccaccacttcagtgatatggtataatttatcaaacagtgttgagaaacagaaactactggggatgttttaaagaggaaaatacttaatatagaaattaggggtttacataatcttaagaaaggatgaaggtgcagctcttagccaggcctccacagtaccacaaaccaacttgcaggaagagctgtaaccactgccccagttgggacaatgggtaatgaggatattaaatttaagaacatactgctatagcaatgatccttggcatagaaagctgccaccacaattgcctagagatgggaacatgaagtctggcccccattgcaacagcagtgaagcagaattttgggactggcatctcccaaatggctttgcttgccaccagagaacaaccaaagtggagggagatggctaggcctcatttctgcctattttattttattttttgagacggagtcttgtctgtcgcccaggctggagtgcagtagtgtgatctcggctcactgcagcctccgcctcccagcttcaaacaattctcctgcctcagcctcctgagtagctgggattacaggcacccgccactgtgcccagccaattttcttatttttagtagaggtggggttttgccacgttggccaggctggtcttgaactcctgacctcaggtgatctgcccgcctcagcctcccaaagtgttgtgattacaggtatgagccaccatgcctggcccatttctcccttttttttttttttttttttttgaggtggagtctcactctgttgcccagactggagtgcagtggtgcaatcttggcgcattgcaacctctgcctcccagtttcaagcaattcttctgcttcagcctcctgagtagctgggactacaggtgtgtagcaccacacctggctaatttttgtttttgttttgttttttttgagacagagtctcactctgtcacccaggctggagtgtagtggcatgatctgggctcactacaacctccgcctcccgggttcaagcaattctcctgcctcagcctccagagtagctgggattacaggtgtgcgccaacacacctggctaatttttttgtatttttaatagagatggggtttcaccatgttggccaggctggtctcgaactcctgacctcgtgatccgcccgcctcggcctcccaaagtgctgggattacaggcatgagccaccgtgcccagacaaggtttgtatttttagtagagacagttttgccatgttggccaggctggtcttgaactcctcacctcaggtgatccgcctgccttggcctcccaaagtgctgggattacaggcgcaagccactgtgcctgacccgtttctgctttttaaagctcatgtgagcacttaatttgtaaccagaatcctacttgtaaaataatctaagacatgtagcttttagctttgtaacctctataatattgatggcacagtgggagtggatgctgagtaccacttgaacatgttccacctcagtgtcttcacagctggaaggtgtctacattgtttcaaggtggacaattgatttacttctcatttttcataaactaaaagtagaataaaggctattcctctaaaattgctatctcacctgtcactcccttgcattctcacataccttcttgagtggaggggcagagggcatggagtgatagcagatgtgccaggaattctccataactcagtccgtccctcttgtgctatgttgcagcatcaggatttgctaatgggaggatactgcccttacgtgcatcattagccatgcacactaaggtcttacacctacacacaggtcagtattctggctcagagaccaacagggagaaattgcagttctcattagttgaactttctttattgttcacagttttaaaacacaaaattgagaggaactctataaaaaatgtgccattctattaataattgttgctggtaatttaaaaatccttgttccttttcaaattcttatataccttttttttttaaacacttgatcttagccaaaagaccgagaagcaatcttttttttttttttttttttttttaacctatagcttctcactgagattgtcagctgtttgtaagttttggtttttggttttctgtgtttgtatttacatatatgaaatacagattgagtatcccttatccaaaatgcttaagactggaagtgttttagatttggggttttttaggatttgtgaatatttgcactatacttaccagttaagcattccaaatccaaaatttcaaatctgaagtgttccactgagcacctcttttgagtatcatgttggtgctcaaaaagtttctgattttggagcatttggatttctgattctcggatttaggatgcttgacctgtaatttcagatttacataaaagcagaaatagtacacagagctccttatatccttcacccagattccccaattattggcctttctgaaccatttgggaataatatgcagatatgattttccattatgtctcagttgttcagtgtatattttctaagtacaagaatatattcctacatatttacatgataaccgtcatgtttaaacattttaaaatggggatttgtattacattgtttctctttttgaaaaaattacagaggagcttaatgcaatcagtattacttaaaatctgataatgtgtgttaaatagtagttttcatttatttcatttatcaggtgttcagtgaatgcttactatgtaacagcacagttatcagcactggggaaatagatgagtaagataagatttgcactttcattagcttacatgccataaagagggaaataaagagaacaccagatgatgataagtttatgctgagaattaaaatgaagtgatgaaataatgggaatgtcaggtggctacttttggtgggatggtcaggaaaggcatctctggggagataaattttaagctcagacctgagtgaaaagaatgagccagccatggaaacattatgttaactcacatggtagtttgaaatgctttatctgatcaaaggtacttatttttggtgactttcaacaatattaagggtctataaaccaacactcatttgcataagaataactaccagtgaatctttttgtatgataggttttttgtttgttgtttttttgagacagagtctcgctctgtcgcccaggctggagtgcagtggcgcgatcttggctcactgcaacctctacctccccggttcaagtgattctcctgcctcagcctcccaaagtagctgggattacaggtgcctgccaccacgcctggctaatttttgtatttttagtagagatggggtttcaccgtgttgtccaggctcgtgtcaaacttctgacctcaagccatccacccgcctcggcctcccaaagtgctgggattacaggtgtgagccaccactcctggccatgataggttattttgtgatgaaaatacctacctcttaatttgtctgataaatttaaattttatgtctagatttcctaagatcagcacttccatattttaaagtaatctgtatcagactaactgctcttgcattcttttaataccagtgactactttgattcgtgaaacaatgtattttccttatgaatagtttttctcatggtgtatttattcttttaagttttgttttttaaatatacttcacttttgaatgtttcagacagcagcaaaagcagcaacagcagcagcagcagcagcagcagggggacctatcaggacagagttcacatccatgtgaaaggccagccaccagttcaggagcacttgggagtgatctaggtaaggcctgctcaccattcatcatgttcgctaccttcacactttatctgacatacgagctccatgtgatttttgctttacattattcttcattccctctttaatcatattaagaatcttaagtaaatttgtaatctactaaatttccctggattaaggagcagttaccaaaagaaaaaaaaaaaaaaaagctagatgtggtggctcacatctgtaatcccagcactttgggaaaccaaggcaggagaggattgctagaacatttaatgaatactttaacataataatttaaacttcacagtaatttgtacagtctccaaaaattccttagacatcatggatatttttctttttttgagatggagtcttgctctgtcacccaggctggagtgcagtgtcgcgatctcggctcactgcaagctctgcttcctgggttcatggcattctcctgcctcagcctcctgagtagctgggactacaggcgcccgccacatcgcctggctaattttttgtatttttagtagagacagggtttcaccatgttagccaggatggtctcaatctcctgacctcatgatccgcccgcctcggcctcccaaagtgctgggattacaggcgtgagccatcacgtccggccagaaatcatgaatattagtaggtgaaaaataaacacattttaccacctggaaaatgaaaaatacttgagtataatctaaataacaatgggaagtgcagagttactttccaggtctcggtttaaatatgtcttaaactttggccaattagtagtagaagttgagagaaaaagtaactatctgacaaagaaattataagcagaatatataaagaactcttaaaactgaataatcagaaaacaactcaataaaaaggtgaaggatttgaaaagatatttcaccaaataagacatagggatgacaaataagcacatgaaaagactctcagcatcactagtcacagggaaatgcacgataaaaccacagtgagacaccatggcacccctgtaggtatggctttaatgaagaaataaaactgacaataccaagtgttggcaaggatccaagcagctgagactcatatactgttaatgggaatgtaaaagtgtacagctttggaaaacagtttggcatttttttgataaatgtatacttagccatgtgatccagcagtcccaatcatgtatatataaccaaaagaaaagaaaacttaggttcacataaaaacttatatcaaatgcttatagctgaccaggcatggtggcccatgcctataatcccagcactttgggaggccgaggttggcagatacctgaagtcaagtgttcgagaccagcctggccaacatggcaaaaccctgtctctacttaaaatacaaaaattagccaggcgtgatggcaggcacctgtagtccagctattcaggaggctgaggcaggagaatcacgtgaacccgggaggcagaggttgcagtgagccgagatcgtgccactatactccagcctgggtgacagagcaaaactctgtctcaaaaaaaaaaaaaaaaaaaagggctggacacggtggcttacgcctgttatcccggcactttgggaggccaaggctgatggatcacctgaggtcaggagttcaagaccagcctggccaacatggtgaaaccccatctctactaaaaatacaaaaatttgctgggcatggtggtgggcacctgtaatcccaggaggctgaggcaggagaatcacttgaacccgggaggcggagattgcagtgagccaagattgtgccattgaactccagcctgggtgacaagaccaaaactccttctcaaaaaaaaaaaagattatagcatctttattcatcattgcccaaaattacaaactgcctaaatgtagaccttcatttagttaatgaatgcacaaactgtggtatatccaaacaattgaataaaaaaaggaatgaactggtacttttttctattcctcctgtttaagtacagccaaaacacctcaacatttgtataaaacatgagctgggctgggtgcggtggctcacacgtgtaatcccagcactttgggaggctgaggcgggtggatcacctaaggttgggagttcaagaccggtctgaccaacatggagaaaccctgtctcaactaaaaatacaagattagtcgggcatggtggcgcatgcctgtaatcccagcttcttgggaggctgaggcaggagaattgcttgatcccgggaagcgaaggttgcagtaagctgagattgcaccattgcactccagcctgggcaacaagagcaaaactctgtctcaaaaagaaaaaaaaaaccattcagctgaatctcaaaggcagagagaagacagactggctagggaccttggaaccagaggagcagtgtggtggggagtggactggattttctttttgcctcatttatcctggacttggtgctggagaagctatgggttcagaccaagagaaaaccccatgaaaagcctgctctctctagccaaaagaggcaacctagcaagataaaaacctttagataataagcacttgactccagtcaaacaaaacagaataaactggccccattcacccctgtcagcaaaggccaagtgggagccaagatatgtaccccaacctggaagtcataaggtacacttctcccctttcccagccaaggtggtgttagagaaggctgactggggagctgggattctcattccctccaggaggtgataacactcctttcacatggtgtcagtggtcacagggaggctgaacttccacccagtaatacataggcatctctctggctcctatatgggtgatgttggagaagaggccgagtagagaatccagactgttgctgacacccagcagtaacaaggacacctccacaatgtccgtggaggccatgtggagatcagtaacaaggcactgctctccctcccagtcagagagatgtcagtggaggactagggggctagaactcccatgtgcgttcagcagtaatccccatgaccgccactccttgacatcacaggccttgaagaaacctggactttcactcccctctggttgtagcgaggtggcactcccttttccctgttgccagtgctgtgtcagtggaggcttgctaaattggaagatgtaaataagattcacattctcataacataataccccaaattttcaggatttaattgaaaatcactaagctgggcatggtggctcacacctgtaatcccagcactttgggaggccaaggtgggccaaacacttaaggtcaggaattcaagaccagcctggccagcatggtgaaaccctgtctctactaaaaatacaaaaattagctgggcgtggtggcacatgcctgtaatcccagctactgggaaggctaaggcaggaaaatcactggaacctgggagacggaggttgcagtgatccaagatcgcactagtgtactgcagcctgggcaacagagcaagactccatctaaatttgtgtcaggattcccagaaggagatgagaaagggtggggctgaaaaaaattgaggaagaagtcatggctgaaaatttcccaaatttggcaaaagtcagaaacctacagattgaaaaagctgaatgaagctcaaatatgataaactcaaagaagttcacacagagacacatcacagtcagatttctgaacactgcagacaaaaaatgaagatctcgaaattagcaagaaatgaccttacctaagcaatttgaatgacagcagatttcccatcagagatcataaaggccagaaggaaggggtacatacaacattttttctagtgctgaaagacaaaaactctaggctgggcacggtggcacacacctgtaatcccagcacttttggaggctgaggcaggcagatcacctgaagtcaggagttcgagaccagcctggccaacatggggaaaccctgtctctactaaaaatacaaaaattagccaggtgtggtggcacgcacctataatcctagctacttgggaggctgaggcaggggaatcgcttgaacctgggaggcgacggttgcagtgagccaaggtcgcgccactgcactccagcctgggcagttgagcgagactccatctcaaaaaaaaaaaaattatccaggcttggtggtgggcgcctatagtcccagctacttgggaggctgaggcaagagaattggttgaacccaggaggtggaggttgcagtgagccaagctcatgccactgtactccagcctgggtgacagagcgagaccttgtctcaaaaaaaaaaaaaaaaaaaaaaaacaagaaaaaaactctaaacccagagttacatatccagtgaaatatccttcaggagtgaagggaaaattaacgatttgtcttcaggagacctaccctaaaagaatggctaaaggaatttctctaaacagaaaagaaatgataaaagaagtaattttggaacatcaggaaggaagaaagaacaataaaaagagtaaaatatgggtaaacacaatagactttcccctccttttgaattttctaaattgtatgatggttgaagcaagaattatagcactgatttggttttcagtatatatattggaaatatttaaggcattatgttacagatgaaggagggtcaaaggatataaagggaggtaacctttctatatttcttttgtactgatgcaggcactttggaaaataatttcactatttgtttaaaaactgaacataccctgaccatatgacatagcatctatactcctgggcatttatcccagagaaacagaaatttatttattttttttttagtattacactccgtaagtgctgtaatactagcacttagggaggctgaggcaagcagattgcttgagcccaggagttcaagaccagcctgggcaatgctgcacagtcaaaaaagaaaaacaaacatttagaaaactattttaaaagtctttaattgctgaatgcctctttggctaatatttggaagatcattattattatttttcttttttaggcagagtcttgctctgtcactgaggctggagtgcagtggcgccatctcggcttactgcaacctctgcctcccgggttcacgccattctcctgcctcagcctcccgagtagctgggactacaggcgtgtgccaccatgcccggctaattttttgtgtttttagtagagatggggtttcactatgttagtcaggatggtctccatctcctaacctcgtgatccgcccacctcggcttcccaaaatgctgggattacaggcgtgagccactgtgcccagcctggaagatcattatttagtcctacaactgacacattgttccactgacgcaattgcccaggctggtcttgaactcctgggctcaagcaatctgcctgcctcggcctccctaagtgctagtattacaggcttgagccactgtgcccagccaaaaatagaaatttatattctcacaaaaacatgtacatgaatgtttatagcagctttacttgtcataatcaaaaactggaaacaaccaaaatgtcctacagtgaaacaaactgtagtacatccatagcatgtaatactctactgtcaggattaaaaagaaacccactgttggcacaggcagcaccgtggctggatctcaggggcattatgctgagtgcaaaaaagcctcaaagggtcttacactgtatgattccacttgttcaactaaaaatgacagctgtatagagatagagaacatattagtggtttccactagttagagaaagtgggtaaaagataggtgggtgggaatataaatcgatagcagggagatctttgtggtattataacacttctatgtcttgattgtagtggtggtggttacatgaatacacgtgtgataaaatgccatgtagaactacatataacgttgtgccaatgtcaatatctaggttttagtttgatctttagttacataagatgtaactattgggtgaaattgggcaaaagagtacacgaaacctctcttaaatatctttacaacttcctttgaattgacagtttttcaaaatagaaagttgggtttttgtaaatacatgaattgttgatatacacaacaaatctcaaatgcattatgctacgtgaaagaagccatattcaaaaggctacatacctactgatgccttttatatgacgtgcaggaaaagataaaactgtaggacagagaatatactggtggctatctgggattaggaaatggggatcgaccacaaaggggcagcatgggggaattttctggggcaatggaatggttgtgtatcttgatggtgtatttgtcaaaatatatagaactataaaagtaaattttgctttatatgtattaaatcaaaaaaagaaactcgtgctcaaatagaaatacattttctgagaacttgccttttgatgactttgagaattttctggaaattttaaagaaatgtggttttgtttcccaacaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttctatttccttttgaatttgtgtttattgttaattcatagctattcaaagtgtgattagagctgggcttggtggcttgcatctacagttccagctacccaggaggcagaagcaggaggattgcttgagcctaggagttcgaggctgcagtgagctatgatcctgccactgaattctagcctgggcgacaaaacaggaaaaaagtatggatggaggaccagcagcatctgtatcacctgtgagtctttcagaaatgcagagtttcaggctacactcggacctactgaatcagaacttgcactttttacaagatccccaggacactaaagtatagagtgaagcttgagaagcgctgttgtgtggattgttcttaaccagctgcagtgatgaatatgaataacgcaggccagcacagtccattgatattctattccagcttactgcctgccaaaaggtccattattactggatcctcagtcttttccaagagaagctaagaattccaaatttttatttgaaatatattttttaaatgtttgttcaactggcccagtgccagtggctcatgcctttaatcctagcactttgagaggccgaggtggaaggatcacttgaccccaggagtttgagaccagcctgggcaacataaagagaccccatctctattaaaaaaaaatagagacaatgctgccttaaaaaagtcaaataaatgtttgctcaactgatttttaatactgagggccaaacaaagcacatcaaatttttaagtgctgcttttcctcattttatccaactctggacaccagaatccaaatgtagtgattggaatccacctagactgattgaggaatatattgtcctcaaattttatgagggttgactattcattttaactttaattaggattgggcacaaattttgaaacataataacattacaggaccggggcccgatccagaccccaagagagggttcttggatcttgtgcaagaatgaattcagggcaagtccataaattgaaagctagttcattaagaaagtagaggaataaaagaatggctaccccataggtagagcagccctgagggctgctggttgccatgtccacccccgcgccccctccccccgctttgttgttgttgtttaagacagagcttcgctgtgtcatcaggctggagtgcagtggcatgatagctcactgcaacctctgcctcccgcgttcaagcaattctcctgcctcagcctcctgagtagctgggactacaggcgtgcaccaccacgcccagctaatttttgtatttttagtagagatgcggtttcaccatgttggccaggaaggtctcaatctcttgacttcgtgatccacccgccttggcctcccaaagtgctgggatgagccacctcgcctggctggttatttcttgatgatatgctaaacaaggggtggattattcatgcctgccctttttagaccatttagggtaacttcccggcattgccatggcatttgtcaactgtcatggtgccaatgggagtgtagcagtgaggaggaccagaggtcactttcatcaccattttggctttttcagccggcttctttactgcaacctgttttatcagcaaggtttttatgatctgtatcttgtgcagaccttctatctcatcctgtgacttagaatgccttaaccatctgggaatgcagcctagtaggtcttggcctcattttaccaaccccctattcaagatggagttgctctggttcaaatgcctctgatatttccctactcccttttataagaaaacccttaatcctaagggttgcagagggatgaagatccatcttctgtattcttcaggctgaataggggtgatgatattcctgcctatgagagtctcttgtattagggtagagaggagctcagtcagtcagtatggctccctatccttcctcccttccccagcccctgacaactaccattctactttgtctctatgattctaagtatctcctataagtggaatcattcagtatttgtagggtttttttgtgactagtgtatttcacttagcatgtcaaggttcatccatgttgtagcatacgtccaaattttcttctttttaaaaactgaataatattccattgtatgatatatgcacattttgcttattcatttatccatcaaagaaaaccagtagcttttttacataccggcagtaacctggttaaaaaaataagatggaaaataccaagcccgaaaatgtcaaattcttgaataccagacaggtactataatccaagctgagcagagaggaatggcccttgtctcagccctgagccacatgtcactacatgggccccaaggagctcttgtgcccagagtcctgctaaggctcgttcttcacccctctaaagtggctcaaactagcgtaggaggccaggagggagcagattctatgccacctccattgcttccgtgccttctccagatccattttttccaaaagctgtgtttgaattctgcacaggggcatctctgtctccaacactgaaaccccttttgccagacccagagaagatttaactgcctttttttttgagacagggcctcgttctgtcacctaagctggagtgcagtggcacgatcttggctcacggcaacctctgcctcctgggctcaagcaattctcctgcctcagccacccgaatagctgggattataggcgcaggccaccacatccagctaagttttgtatttttagtagagacgcagttttgccatgttgcccaggctggtctagaacttctgagctcaggtgatccacccgcctcggcctcccaaagttctgggatgacaggcatgagccacctaacctggccagatttaactgctttgagccaccgtctcacacatcttccaggagcttctagtcaaatgcagaggattaatagtgtataattccatttacataagatctttggaatagtcaaactcatagagacagaaagtagaatggtggttgccgggggatggttgaggggaaaattaggagttgtttaacgagtatggagtttcagtttgggaagatgaaaaaagttctggagatggacagttgtaatggctgcacaacaacgtgaatgtacttaatgccggtgaaccgtataaccatttaacaactgaaaatggttacaaaattttgtgttatatatatcttatcacaataaaaaatatagaggattgatcactatatttgctcccttccaaaatctcacctaggttaaagtaaagaaaaatatataaacccacaagaacacagggaccaggagaggagatgcattcattggattacgtttggttgtaaaaaaaacaaaaaatcctccccggcacggtggctcacaccttgtaatcccagcactttgggaggctgaggcaggcggatcacgaggtcacgagtttgagaccagcctgaccaacatggtgaaacctcatctctactaaaaatacaaaaattagccgggtgtggtggcgtacgcctgtaatcccagctactgaagtggctgagacaggagaattgcttgaacccgggaggcggaggttgcagtgagccgagatcacgccactgcactccagattaggtgacagaacaagactccatctcaaacaaaaacaaaaacaaaaacaaactaatataacagtgactttacatgctatgggttttttttctcctctcatcttcaagaaatgtggagactgccagtccggggatgatacagcatcacagtgtcaggaatctgctctttctgtataattgttacaggaaaggggtccggatccagaccccaagagagggttcttgaattcagggcaagtccacagtgcaaagtgaaagcaagtttactaagaaagtaaaggaataaaaaaatcgctactccatagacagagcagccctgagggctgctggttgcccatttttatggttatttcttgatgatatgctaaacaaggggtggattattcatgcctcccctttttagaccatatagggtaacttcctgatgttgccatggcatttgcaaactgtcatggtgctggtgggggcgtagcaccaaggaggaccagaggtcactcttgtggcctttgtggttttggtgggttttggccagctcctttactgcaacctgttttatcggcaaggactttatgacctgtattttgtgttgaccttgtatctcatcctgtgacttagaatgccttaaccatctgggaatgcagcccagtaggtttcagcctcattttacccagctcctattcaagatggagttgcagccgggcacggtggttcatgcctgtaatcccagcactttgggaggttgaggcgggtggatcacaaggtcaagagttcaagaccagcctggccaacatggcaaaaccccatctctactaaaaatacaaaaattagccaggcgtggttggtgtgcgcctattatgccagctacttgggaggctgatgcaggagaatcacttggatctgggaggcggaggttgcagtaagcagatatcgcgccatgcactccagcctggatgacagagcaagactccgtctcgggggaaaaaaaaaccaaaatggagttgctctggtttacaggcctctgacataatggctttatgtacatggcttccatggctagggtcagctcatagatctaatatggctgtgttggtgctcatgttgcagcccagcaatcagaaagagaaaggaggctgggtttggtggcttacacctgtcatcccagcatttcgggaggccaaggcgggaggatcacatgagcccatgagtttgagaccagcttgagcaacacagggagacctcatctctacaagaaataaaaataaattagttgggtatggtggcatgcacctgtggtcccagctattcgggaggctgaggtgagaggataccttgagcctggaggtttgaggctgcagtgagctatgatcgcaccactgcactccagcctgggcaacagagtgagaccccatctcaaataattaaaaaaaaaaaaaagaaagaaagaaaaaaaaaaggagagcagaaaaaggctatgtaccttttctttcgggttaccttctcaaagttgtgcatactacttccgtttacattacattggccagaacttaacatgacaactactagctacaaggtggtttttattctgggttgccatgcatcttagcttaagtaccctacaggtcctgagataatgattcctatgaaaatgattatttacttatttaattaatttattttgagatggagtctcactctgtcacccaggctggagatcagtggcgtgatctcggctcactgcaacctctgcctcccaggttcaagcaattctcctgcttcagtctcccgagtagctgggactacaggcatgcgccaccatgcccagcttttttgtatttttagtagagacggggtttcactatgttggccaggctgatctcgaactcctgacctcaggtgatctgcctgcctcggcctcccaaagtgctgggattacaggcgtgagccactgtgcctggctgaaaatgattttttaaaagtgttccaggaggaaatggaaagggcataggggagtaagaaagtggaaataggaaaagaaggaagccaagcaagaggagcctatcaagcaaagcctcagtcctgcagacagtctggaggcaggatattcacatcttagagttgtccaaaacagaacaggcaagctgcataattttcaactctggctgaataaggttgcatttcagctctctgggcacctggaaagtatgggctccagtaacctggaggcagtctgctgactgctggctggtgtgcacaaaagtggtaaagggatgagggagtgcaatatgggcactggcatcttggctgtcatgcatccaggtaaaaagtggaggctcaattactatatatgaaggggagaacagactttggggaacaattagcagtcatttccaggtgagacaacaacagaagagaaatgctaatttttttttttttctttgagatggagtttcgctcttgttgcccaggctggagtgcaatggcgctatatccagctcaccgcaacctccgcctcctgggttcaagtgattctcctgcctcagcctcccgagcagctgagattacaggccaccacacccagctaattttgtatttttagtagagacggggtttctccatgttggtcagggtggtctcaaactcccaacctcaggtgatccgcccgcctcagcctcccaaagtgctgggattacaggcgtgaaccaccgcacccggcgagagatgcgaacattttttttgttttttggaggatgacaagttgatgcaggaatgagcagaaccaggaggctgaatactgagtgttaaagagttcctagcaatatgcaggaaaggaaagcaacaagtatcctgagaggtgggggttagatgtagactgaaatcaggaggtgtggttaaaactctttgaaagaggctgggtgcggtggctcacataatcccagcactttgggaggccaaggcgggtgcatcatgaggtcaggagttcgagaccagcctgaccaacatgtgaaaccccgtctctactaaaaatacaaaaattagccgggcatggtggcgcatgcctgtaatcccagctactaaggaggctaaggcaggagaaccgcttgaacctgggaggcgaaggttgcagtgagccgagatcatgccattggactccagcccaggcgacagagagagatcccatctcaaaaaaaaaaaaaaaaaaaaaaaaaaagaaaggaaaagaagaaaagaaaaaagaaacagcagattccctagagaggagacattcatcactaaaaaaaaaaaaaaaaattaaaataaatcgcagaagtaaaagccctttttgaaagggctcagggtacccagcttaatgtatgaaacaaaagcaaacccacatcaaagcacaccattattaatttcagagcatcagaaataaagagaagatctgacaagcttcttggaagaaaacaaggcatagagaataatcagagtagagtagagcaggaaaacagagtcctgggtagagcgaactgagcctagccaatcgatagaattcttgatgttttgttgaaaacagaagcactttacaattctcttggtaagttttagaaagattctgtagtaatggaaaattgtacaaatttaaaaaatggaatctgttaacttcaggaagaacaaaaggtagaaaaatcaaggcaatataatcttggtacaacatttgacttagctgcaatgaataagacttacatagtcatagaaggtaaacactgattacttactttaccaaaaattatgatatcatcatattgaaagcccagaaggaagggaaataaagggaaatacaagagtgctaaatcttttatttttcaataagttggtaatgtctaaaattaataagaaaatggcaaccataagcctaatgcttagaaattcagaaaagtatcagactaaattttaaagtctactttaaactgagtctttgctttatagttgcataaataaattcattttgatggttttctcagaacagatgaactggctgggtgcggtggttcatgcctgtaatcttagcactttgggaggccaaggccgatggctcacttcaagtcaggagtttgagaccagccaaaaaattagccgggtgtggtggtgcacgtttgtaataccagctactcaggaggctgaggcaggagaatcgcttgaacctgggaggcggaggttgcagtgagccaagatggaacgactgcactccagcctgggtgacacagtgagactccgtctcaaaaaaacacaaagaacagatgaactgttttcctttgaggaatattgatgatcccactgcttgagtaatggaaaccagcttgcagctgatagttattttttgtcttttttcctgttctatccactgtcctaggtttggaatttcagctgggcagcttcgatcccaccacctgtagccactgaggttcacaggctctgtttgaggtgttgtgttagattgtgctgggttggttttgctgcttattgagtgatgaagagcaacatattagctactgagatattttgcagaaaaaggaagagatgtcttcagctcaaatgctatcaaacttgtatgtcttttaactttttattataaaaaatttcaaacatatgcaaaagcaaaagactagtttaatgaaccttatgtacccaccacccagtttcaacaattatcagttcctgacaattcttgtttcacctatcccacctctattatacacacatacaaacacacagacaccacacacacacacacacacacacacacgagagcaaagtcaacttccattattttgaagcacatccaagacatcacatcacattatttatcacattatttattaagtttcttcagtcacagtccatctcacagaaagaaatcccccatgagaaacttttaggacatagcttttatgttaattacttaatttctgcactagccatccttgcctgccttctaattttctgcactagctatccttaccttccttctaattttctaacctgccaaggattctgctgcctcaaggtatttgtttctttctgcctggattgctctttcgcagatacttacatgtctcattctttttcacccaggtgaaataaacagcgttgttgctcacacaaagcctgtttggtagtctcttcacatggacgcgtgtgacatttggtgctgaagacccgggacaggaggactccttcaggagacgggtcccctgtccttgccctcactccgtgaggagatccacctacgacctcgggtcctcagaccaaccagcccaaggaacatctcatgaatttcaaatcgggtaagcggtcttttccatcctctcttgctacccttcaatctccctctctcgctacccttcaatctccctgtccttccaattccagttctttttcctctctagtagagacaaaggagacacattttatccgtggacccaaaattccggcgctggtcacggactcaagaagacagtcttctcttggtgtttaatcactgcagggacgactgcctgattattcacccacactccattggtgtctgatcaccgtgggggtgcctgtcttggtcattcacccacattcccttggtggcaagtcaattgcggggacgcctgctttggctgctcacccacccccttctccgtgtctctacctttctctttaaacttacttccttcactatgggcaacattccgtcctccattcccccttctactcccttagcctgtgttctcaagaacttaaaacctcttcaactcacacctgacctaaaacctaaaagccttattttcttctgcaataccgcttggccccaatacaaactcaacagtagttccaagtggccagagaatagcactttggatttgtctgtcctacaggatctagataatttttgttgaaaaatgggcaaatggtctgaggtgcctcagatccaggcattcttttacacactggtccctccctagtctctgctcccagtgcgactcatcccaaatctttcttctttctctcctgtctgttccttcagtctccaccccaagctccgagtcctttgaatcctccttttctatggactcatctgacctctcccctcttccccaggctgctcctcaccaggctgagccaggtcccaattcttcctcagcctccggtcccccaccctataatccttttatcaccccctcctcacaccgtgtctggcttacagtttctttctgtgactagccctcccctacctgcccaacaatttcttcttaaagaggtggctggagctgaaggcatagccaagggtaatgctcctttttctttatctgacctctcccaaatcagttagcgtttaggctctttttcatcaaatataaaaactcaacccagttcatggcctgtttggcaacaacccttggacactttaccgccctagacccagaatggccagaaggccgtcttattctcaacatgcattttattacccaacccactcccgacattagaaaaagctccaaaaattagattccggccctcaaaccccacaacaggacttaattaacatcaccttcaaggtgtacaataatagagtagaggcagccaagtagtaacgtatttctgagttgcaattccttgcctccactgtgagacaaaccccagccacatctccagcacacaagaccttccaaatgcctgaactgcagcggccaggcattcctccaggactgcctaccccaggatcttgcttcaagtgccggaaatctggccactgggccaaggaatgcccccagcccaggattcctcctaagccatgtcccatctgtgcaggaccccactggaaatcggactgtccaacccggcagccactcccagggcccctagaactctggccaaaggctctctgactgactccttcccagatcttctcagcttagcagctgaagactgatgctgcctgatcgccttggaagccccctggaccattacggatgctgagctttggataactcttacagtagagggtaggtccatcccctgtttaatcgatatgggagctacccactccacattaccttcttttcaagggcctgtttcctttgcccctataactgttgtgggtattgatggccaggattctaaaccccttaaaactcccccactctggcaccaacttggacaacattcttttatgtactcttttttagttatccccacctgcccagttcccttattaggccaagacattttaaccaaattatctgcttccctgattattcctggactacagccacatctcattgccacccttcttcccaacccaaagcctccttcgcctcttcctctcgtatcccccaaccttaacccacaagtatgggacacctccactccctccctggcaaccgatcaaacacccattactatcccattaaaacctaatcacccttacctggctcaatgccactattccatcccacaactggctttaagaggactgaagcctgttatcactcgcctgctacagcatgggcttctaaaacctataaactcttcttacaattctcccattttacctgttcaaaaaccggacaagtcttacaggttacctcaggatctggatcttatcaaccaaattgttttgcctatccaccctgtggtgcccaacccatacacttgtttgtcctcaataccttcctccacaactcactattccgttcttgatctcacagatgcttttttcactattccctacaccactcatcccagcctcttttgcttttacctggactgaccctgacacccatcagtcccagcagcttacctgggctgtactgccgcaaggcttcagggacagccctcattacttcagccaagctctttctcatgatatactttctttccatcccttcgcttctcaccttattcaatatattgatgaccttctactttgtagcccctcctttgaatcttctcaacaagatacttttcaacatttattctccaagggatatcggatgtccccctccaaagctcaaatttcttctccatccgttacctacctcagtataattcttcataaaaacacatgtgctctccctgctgatcatgtctgactgatctctcaaaccccaacaccttctacaaaacaacaactcctttccttcctgggcatggttggatacctttgcctttggatacctggttttgccatcctaacaaaaccattatataaactcacaaaagcaaacctagctgacctcataaatcctaaatcctttccccactcccctttccattccttaaaaaacagccctaaaagctgctcccacactagctctccctaactcatcccaacccttttttcattacacacagctgaaatgcagggctgtgcagttggaattcttacacaagagccaggaccatgccctgtagcctttctgtccaaacaacttgaccttactgttttaggctagcccccacattattcctgataccacacctgacccccatgactgtatctctctaatccacctggcattcactccatttccccatacttccttctttcctattcctcaccctgatcacacttggtttattgatggcagttccacctggcctaatcgccattcaccagcaaaggtgggctatgctatagtagtatcttccacatctatccttgaagctaccgctctgcccccctccactacctctcagcaagctgaactcattgccttaactcgagccctcgctcttgcaaaaggactacatgtcaatatttatactgactctaaatatgccttccatatcctgcaccacaatgctgttatatgggcagaaagaggtttcctcagtacgcaaaggtcctccatcattattgcctccttaataaaaactcttctcaaggccgctttacttccaaaggaagctggagtcattcactgcaagggccatcaaaaggcatcagatcccatcgctcagggcaatgcttatgctgataaggtagctaaagaagctgctagctttctaacttctgtccctcacggccagtttttcttcttctcatcagtcactcccacctattcaccgactgaaacttccacctatcaatctcttctcacacaaggcaaatggttcttagaccaaggaaaatatctccttccagcctcacaggcccattctattctgttgtcatttcataacctcttccatgtaggttacaagccactagcccacctcttagaacctctcatttcctttccatcatggaaatctatcctcaaggaaatcacttctcagtgttccatctgctattctactacccctcagggattattcaggccccctccctaccctatacatcaagttcagggatttgcccccacccaggactagcaaattgactctattcacatgccctgagttaggaaactaaaatacctcttggtctgggaagacactttcactggatgggtagaggcctttcccacagggtctgagaaggccaccacagtcatttcttcccttctgtcagaaataatttctcggtttggccttcccacctttatacagtccgataacggactggcctttactagtcaaatcacccaagcagtttctcaggctcttggtattcagtggaaccttcataccccttaccgtcctcaatcttcaggaaaggtagaacggactaatggtcttttaaagacatctcaccaagctcagcctccaacttaaaaaggactggacagtatttttacctcttgcccttcacagaattagagcccgtcctcgagaagctacagggtacagtccatttgaacttttatatggacgcactttcttgctcagccccaacctcgttccagacaccagccctctaggcgactatcttccagtcctctggcaggctagacaggaaatccaccaggctgctaatcttctcttgtctactccagattcccaactatatgaagacaccctagctggacgatcagttcttattaagaacctgacccctcaaactctacaacctcgatggactggaccctacttagtcatctatagtaccccaactgccgtccgcctgcaggatcctccccactgggttcaccgttccagaataaagctgtgcccatcggacaaccagcctaatctctcttcttcctcctggaagtcacaagtactccccactacttcccttaaagtcactctcatttctgaagaacagtaataacccttatgagcctaatacatcccttcattctattagctctattcatccttaccctactttttgcaacagggctttacacagtcaccccccctacttggactgaaccccaaaaacttgtcatccctactatcttctgtttactcatactcccattcactattctcaactactcataaatgccctaatcttgtttacattgcctgtttacactgtttctctaagccatcacagctggtatctcctggtgctatccccaaaccgccactcttaactccctcttagagtggatagatgatatttgctggcagggcaacctccaatattttcactctgatgaagttctattctttacttttatactcactcttattctcattcccattcttatgccaccctctacctctccccagctatctccaccacactatcaatcttactctctcctagccgtttctaatccctccttagcgaataattgctggctttgcatttccctttcttcctgcacctacacagctgcccccgccttatatacagactgggcaacatctcctgtctccctacacctccaaatttcctttaacagccctcatctttaccctcctgaagaacttcttcactttctagacaggtccagcaaggcctccccagacatttcacatcagcaagctgccgccctcctccacacttacttaaaaaacctttctccttatatcaactctactccccccacatttggatccctcacaacacaaactacttttcctgtggctgctcctttatgtatctctcagcaaagacccactggaattcccctgggtaacctttcaccttcttgatgttcattcactcttcatctccaaagcccaactacacacatcactgaaacaactggagccttccagctccgtattacagataagccttttatcaatacaggcaaacttaaaaacattagaagtaattattgcttaggaagacaccctgtatttcactccatccttggctaccttccccttgctcgtcagaccctcctcccaggccttcttgttatacccagccccgtaaataacagtgaaaggttgctcatagacactcgacgttttctcatacaccatgaaaattgaacctctccctctacacagttaccccatcagtccccattacaacctctgacggctgccgccctagctggatccctaggagtctgggtacaagacacccctttcagcactccttctcatctttttactttgcatttccagttttgcctggcacaagctctcttcttcctctgtggatcctctacctacatgtgtctacctgctaactggacaggcacatgcacactcattttccttaatcccaaaattcaatttgcaaatgggaccaaagagcttcctgttcctctcatgacaccgacatgacaaaaaagggttattccactaattcccttgcttgtcggtttaggactttctgcctcctctattgctctcggtactggaatagaaggcatttcaacctctgtcacaaccttccatagcctctctaatgacttctccactagcatcacagacatatcttaaactttatcagtacttcaggcccaagttgactctctggctgcagttgtcctccaaaactgccaaggccttgacttactcactgctgaaaaaggaaggcgctgtgtgtatgtgtgtgtgtatatatatatatatatatatatatatatattttttttttttttttttttttttttttttgagacggagtcttgctctgtcacccaggcagtggcgcatctcggctcactgctcactgcaagctccacctcctgggttcatgccattctcctgcctcagcctcccaagtagctgggactataggcgcctgccaccacgcccggctaattttttgtatttttttagtacagacagggtttcaccatgttagccaggatggtctcgatctcctgacctcatgatccgcctgcctcggcctcccaaagtgctggggttataggcgtgggccactgcgcccggccgggctctgtatatttttaaatgaagagtgttctttttacctaaatcaatctggcctggtgtatgacaatataaaaaaactcaaggacagagtccaaaaacttgccaaccaagcaagtaattatgctgaacccccttgggcactctctaatcggatgtcctgggtcctcccaattcttagtcctttaatacctgtttttctccttctcttatttggaccttgtgtcttccgtttggtttctcagttcatccaaaaccatatccaggccatcaccaatcattctatatgacaaatgctccttctaacaaccccacagtatcactccttaccacaaaatcttccttcagcttaatctctcccactctaggttcccacaccacccctaataccgctcgaagcagccctgaggaacatcgcccattatctctccatactaccccccaaaattttcaccactccaacacttcaacactattttgttttatttttcttattaatataagaagacaggaatgtctggcctctgagccaaggcctgcatgtatacatccagatggcctgaaggaactgaagaatcacaaaagaagtgaaaatggctgattcctgccttaactgatgacattaccctgtgaaattccttctcctggctcagaagctcccccaccaagcaccttgtgacccccccgcccctgcctgccagagaacaaccccctttgactgtagttttccactacccacccaaatcctataaaactgccccacccctaacaccctttgctgactctcttttgggactcagcccacctgcacccaggtgaaataaacagctttattgctcacacaaagcctgtttggaggtctcttcacacggatgcccgtgacaagtgcctgccaccacatcccactaatttttgtatttttagtagagatggggtttcaccatgttggccaggctggtcttgaacgcctgacctcaggtgatccacctgcctcggccttccaaagtgctgagattacaggcgtaagcccctgcacccggccctgttttttttttttttttttttttttacatcccggagtcacactggatatctgaatgtgcttaaaataaaattcacatctcattctggaagtgtttattaaatgctgtccctgcattcatgtgaacatcacagcccagcggttagatgtgcagactccaacgcctgactgcctgggtctgtcacttactagctgtatgaccttggatgggttacttaacatttctgggcactggtttctcattagtaaaatgaagaatgggcgcctgtagtcccagctacttgggagcctgaggcaggagaatcgctttaacccgggagacgaggttgcagtgagcctaggtcccgccattgcactccagcctgggccacagagcgaggctccatctcaaaaaataaaatttaaaaaaaaaaaattcagctttcacggagaattactactgcctggtgccatcagatcccaaatactggaggcattcccaatgttctgttcacaaactgtgcaatctgtgatagcccctcttgtgaatataatcaaagtgaaagatgccagtatacttattgctatgcttaaaagaactgcagatctatcatatccttttgaaaccaaaaaatgtggaagtctccctggctagatgctgtgtgtccacgtatcctgcaccaattgcttgaaagtcacatgacaacctttcttcctttagggaaggaagcaagcaaggtaggactctgtgtgttacagccagtgtgggtaactaaggactgagggaggtgtgggcacttgcatagctagtgccgccaataccataggagcgttgacactaaacggcacttctctattaagttgccctgctgtcttctaacgagtcattttgaaaaacggttgtccgtttttcaactacttgggtgagggcagcttcatgaaaaaagctcatcgtatgttaaagaaggtagggtttcaattgtgagctataaagccaccagaggagatttctcctatagaaggaaggtgtgtttcctgcttggacacctgaatttgcaacccatcctttcttttttcctttttccttttttttttttgcaacccatccgttctatctttgtagcctcagctcctaccatagtgcctgtcacggagcagctaatcgacacagtgacgcgtgtaagttatttgaagccacttcctgcgcagtggaacgtccccgccaagtctcccggcactgctgggtgtagtcccaattaaaacactaggtttcaacaacacaaggagggtccgcagggtccccggactacccaccaagggtcggctctgctttgaaacgcagctgcaggaggccgaggagcgccgcgatgccccagttatccaggccattccttccccggccgaagctggccgcctgcatgcacctaaccttgcgctaggcactggctgcggttaatccagcccgcgctcggtcctccgcgatcccagcctcccttgcgccaaggacacgtccggcggcttcgcggggcctgtctacgccaccgcggcgcttttctatgatccgctgccgctttccgagcgagtgtcatggcggccggcgtcgagttggcaggagtaacccacggaactgaggaaagtcattagagctgagaaagaagtggcccaatctggacggtgggaattcgtgggaatgagcagaaggccctccgtaggtgactgtgtcactagaggcgggcccctggtaaaattccaggccaggcctctgcgtttctaggcagaacctggagtcggccttgcctgagaacccagctttgtgttatcgtatcctgtctcgcgaaggcaggcgttcaaggatatttggtcggatcgcccggcggcgctaaacgttttcttttttccgagcggaccgggtcgttctctaaactcgccgcgatgtcgtcctggcttgggggcctcggctccggattgggccagtctctgggtcaagtcgggggcagcctggcttccctcactggccagatatcaaactttacaaaggatatgctgatggagggcacggaggaagtggaaggtaacagctggagcgagggaagggagggcttttctaagaccctgggaacgtccgtttggtccctacttccatcagtcaggagacgcggacaggtcgcaaaggaatgctgctcctcccttttttcttcacacacctcctcttgcctcttctcaacagattttctatgatgaaagaccactgggggccaggggaagagtcctggttctcctctctttaaatattctggatatctcaaagtacagtagagtgaaactgctcctacgttaagtcgagtttattttcgtttggttgatccacgcccaccactttgccctcagtttgggggctgatctcattcttaacatcttcctgtattctgacccagtttaaagaattgtatatctgttttctatttttgtaacagttgaaataactcataatttaaacacttggtgagttttgctcttttctgtcttgagtatgttatgaaacacggccatgtttttctatttaattattttttacttttccttattgtgatgatacattatagcgttccagctgagaatgtatttttgtgtataatcgcccatttcgcccatatttcgacagtagaatagggaagacaaccctattcttaaggtacccttagcaatcttcactcatttttgaagtcctcaatctcgttttactaatgaatagtgacccagagttacatggtaatcaatggctacattagttactatccattaattactattttgatttcttggcattttagtaagcaatgtttggatcaaaacagtttttaagtttcaatctaagtctgctccagggagataatgggctgttcttttgggtgagagctgctgctttgacaattgtatttcttataaataaatttaataaattagtattctggcttagtttttttttttaattgctaatttaacagatccttcatatttatttggtagaaatcgggaaagattgtttgatagaaagataatgcttatgttttgaatttctgtggggtctatttagatggaaggaagaatagactggggagctaccgaaatccattttaccgctttacgttccccatgccataaaggtgcatcagtgagacagctttggggatagaaatgaaagggcttctttatgtgtgggggaaagcatgggaccttgagggcagtcagaagcacagaagggctggataactatgggaaatgactggtcgctccttgtatcctagataggggaaaacggttggctgttccttagaccctaagccagagaatatgcatccctcaacctggatgtcttccaggtttaaggtggtgttcattttgttctcctgccaaatgtgctgttttctcattcttaatagcttcactctcagtttcctaaactgtaaatcttcgtgtccttgttttatgtcttttttcatctcctacttccagttggtcatgatatcttgtttatcctatgccagaattgtcctttgctttattactccttattctttagatccctataatctttcccagggactattcctctttcatgtcctgtcacccatttctccctaccccagtccattttctattgtattgtcagttactttctaaaaacaaacctgcgtaaaaatttcttgaccctgttacagaataaaatccaaacctcttagttttggactgagtctcactctgttgcgtaggctggatgcagtggcgcgatctcggctcgctgcaaccttcgcctcccgggttcaagcaattctcctgcctcagcctcccaagtagctgggattacaggcgtgtgccaccacacctggctaatttttgtatttttagtagagatggggtttcgccatgttggccaggctgatctcgaactcctgagaccgcaggtgatcctcctgccttggcctcccagagtgctgggattacaggcgtgagccaccgcacccgaccttcaatgtaccttttctagttgccataccagttgctgtacatgtgcacatatatatgcttgcttgtacgcacagcttctgtcgtgtgggattttgtcaggcacttacacaacttcaagcctctacatggaaagccctttcttctctatttcctccttcacccagaaaacaattctcctgtacaaccctgctcatatgtcaggtacttaagtacttggtgaaccattctgactcccacacaggttgagttgttgagttgaatgttggtgatctgtgttctaacagaactttgtggatctctccctgacagcatttaacatgttgagttataatttatctgttttcctgatgcccagcgtgaggactgaatttttttgctccttatattaccaaatatgtgacaccttccttttggcacatagtagatgctcaataaaaatggcacaagtgcgtgattagaaatctgcaaagttgcttaatgcctttattctttcccctttgtatttcaatcggacttgcttaacacctaatttattgattttgtaaatttttaaaaataggggtctggccaggtgtggttcatacctgtaatctcagcattttgggaggctaaagtgggaggatagcttaaagccaggagttgaggactagcctgggcaacatagggagatctcatctctacaaaaagtttttagctgggcatggctgcatgtgcctgtagtctcacctacttgggaggctgaaatgggaggtttgcttgagcccagtagttaaaggctccagtgagctgcgattgcaccactgaactccagcctgggtgacagagacgaccctgtctaaaaaaataaagggaatggagggtcagagtacagttcttttattcattgaacaagtatttattagtgtttaccctgtgtcagtaatgaaaaataaaacagttc

SEQ ID NO: 2 - DNA, 6923 bp, Homo sapiens
gagaggggcagggggcggagctggagggggtggttcggcgtgggggccgttggctccagacaaataaacatggagtccatcttccacgagaaacaagaaggctcactttgtgctcaacattgcctgaataacttattgcaaggagaatattttagccctgtggaattatcctcaattgcacatcagctggatgaggaggagaggatgagaatggcagaaggaggagttactagtgaagattatcgcacgtttttacagcagccttctggaaatatggatgacagtggttttttctctattcaggttataagcaatgccttgaaagtttggggtttagaactaatcctgttcaacagtccagagtatcagaggctcaggatcgatcctataaatgaaagatcatttatatgcaattataaggaacactggtttacagttagaaaattaggaaaacagtggtttaacttgaattctctcttgacgggtccagaattaatatcagatacatatcttgcacttttcttggctcaattacaacaggaaggttattctatatttgtcgttaagggtgatctgccagattgcgaagctgaccaactcctgcagatgattagggtccaacagatgcatcgaccaaaacttattggagaagaattagcacaactaaaagagcaaagagtccataaaacagacctggaacgagtgttagaagcaaatgatggctcaggaatgttagacgaagatgaggaggatttgcagagggctctggcactaagtcgccaagaaattgacatggaagatgaggaagcagatctccgcagggctattcagctaagtatgcaaggtagttccagaaacatatctcaagatatgacacagacatcaggtacaaatcttacttcagaagagcttcggaagagacgagaagcctactttgaaaaacagcagcaaaagcagcaacagcagcagcagcagcagcagcagggggacctatcaggacagagttcacatccatgtgaaaggccagccaccagttcaggagcacttgggagtgatctaggtgatgctatgagtgaagaagacatgcttcaggcagctgtgaccatgtctttagaaactgtcagaaatgatttgaaaacagaaggaaaaaaataatacctttaaaaaataatttagatattcatactttccaacattatcctgtgtgattacagcatagggtccactttggtaatgtgtcaaagagatgaggaaataagacttttagcggtttgcaaacaaaatgatgggaaagtggaacaatgcgtcggttgtaggactaaataatgatcttccaaatattagccaaagaggcattcagcaattaaagacatttaaaatagttttctaaatgtttctttttcttttttgagtgtgcaatatgtaacatgtctaaagttagggcatttttcttggatctttttgcagactagctaattagctctcgcctcaggctttttccatatagtttgttttctttttctgtcttgtaggtaagttggctcacatcatgtaatagtggctttcatttcttattaaccaaattaacctttcaggaaagtatctctactttcctgatgttgataatagtaatggttctagaaggatgaacagttctcccttcaactgtataccgtgtgctccagtgttttcttgtgttgttttctctgatcacaacttttctgctacctggttttcattattttcccacaattcttttgaaagatggtaatcttttctgaggtttagcgttttaagccctacgatgggatcattatttcatgactggtgcgttcctaaactctgaaatcagccttgcacaagtacttgagaataaatgagcattttttaaaatgtgtgagcatgtgctttcccagatgctttatgaatgtcttttcacttatatcaaaaccttacagctttgttgcaaccccttcttcctgcgccttattttttcctttcttctccaattgagaaaactaggagaagcatagtatgcaggcaagtctccttctgttagaagactaaacatacgtacccaccatgaatgtatgatacatgaaatttggccttcaattttaatagcagttttattttattttttctcctatgactggagctttgtgttctctttacagttgagtcatggaatgtaggtgtctgcttcacatcttttagtaggtatagcttgtcaaagatggtgatctggaacatgaaaataatttactaatgaaaatatgtttaaatttatactgtgatttgacacttgcatcatgtttagatagcttaagaacaatggaagtcacagtacttagtggatctataaataagaaagtccatagttttgataaatattctctttaattgagatgtacagagagtttcttgctgggtcaataggatagtatcattttggtgaaaaccatgtctctgaaattgatgttttagtttcagtgttccctatccctcattctccatctccttttgaagctcttttgaatgttgaattgttcataagctaaaatccaagaaatttcagctgacaacttcgaaaattataatatggtatattgccctcctggtgtgtggctgcacacattttatcagggaaagttttttgatctaggatttattgctaactaactgaaaagagaagaaaaaatatcttttatttatgattataaaatagctttttcttcgatataacagattttttaagtcattattttgtgccaatcagttttctgaagtttcccttacacaaaaggatagctttattttaaaatctaaagtttcttttaatagttaaaaatgtttcagaagaattataaaactttaaaactgcaagggatgttggagtttagtactactccctcaagatttaaaaagctaaatattttaagactgaacatttatgttaattattaccagtgtgtttgtcatattttccatggatatttgttcattacctttttccattgaaaagttacattaaacttttcatacacttgaattgatgagctacctaatataaaaatgagaaaaccaatatgcattttaaagttttaactttagagtttataaagttcatatataccctagttaaagcacttaagaaaatatggcatgtttgacttttagttcctagagagtttttgtttttgtttttgtttttttttgagacggagtcttgctatgtctcccaggctggagggcagtggcatgatctcggctcactacaacttccacctcccgggttcaagcaattctcctgcctcagcctccagagtagctgggattacaggcgcccaccaccacacccggcagatttttgtatttttggtagagacgcggtttcatcatgtttggccaggctggtctcgaactcctgacctcaggtgatccgcctgccttggcctcccaaagtgttgggattacaggcatgagccactgcgcctggccagctagagagtttttaaagcagagctgagcacacactggatgcgtttgaatgtgtttgtgtagtttgttgtgaaattgttacatttagcaggcagatccagaagcactagtgaactgtcatcttggtggggttggcttaaatttaattgactgtttagattccatttcttaattgattggccagtatgaaaagatgccagtgcaagtaaccatagtatcaaaaaagttaaaaattattcaaagctatagtttatacatcaggtactgccatttactgtaaaccacctgcaagaaagtcaggaacaactaaattcacaagaactgtcctgctaagaagtgtattaaagatttccattttgttttactaattgggaacatcttaatgtttaatatttaaactattggtatcatttttctaatgtataatttgtattactgggatcaagtatgtacagtggtgatgctagtagaagtttaagccttggaaataccactttcatattttcagatgtcatggatttaatgagtaatttatgtttttaaaattcagaatagttaatctctgatctaaaaccatcaatctatgttttttacggtaatcatgtaaatatttcagtaatataaactgtttgaaaaggctgctgcaggtaaactctatactaggatcttggccaaataatttacaattcacagaatattttatttaaggtggtgctttttttttttgtccttaaaacttgatttttcttaactttattcatgatgccaaagtaaatgaggaaaaaaactcaaaaccagttgagtatcattgcagacaaaactaccagtagtccatattgtttaatattaagttgaataaaataaattttatttcagtcagagcctaaatcacattttgattgtctgaatttttgatactatttttaaaatcatgctagtggcggctgggcgtggtagctcacgcctgtaatcccagcattttgggaggccgaagtgggtggatcacgaggtcgggagttcgagaccagcttggccaaaatggtgaaaccccatctgtactaaaaactacaaaaattagctgggcgcggtggcaggtgcctgtaatcccagctacctgggagtctgaggcaggagaattgcttgaaccctggcgacagaggatgcagtgagccaagatggtgccactgtactccagactgggcgacagagtgagactctgtctcaaaaaaaaaaaaaaaatcatgctagtgccaagagctactaaattcttaaaaccggcccattggacctgtacagataaaaaatagattcagtgcataatcaaaatatgataattttaaaatcttaagtagaaaaataaatcttgatgttttaaattcttacgaggattcaatagttaatattgatgatctcccggctgggtgcagtggctcacgcctgtaatcccagcagttctggaggctgaggtgggcgaatcacttcaggccaggagttcaagaccagtctgggcaacatggtgaaacctcgtttctactaaaaatacaaaaattagccgggcgtggttgcacacacttgtaatcccagctactcaggaggctaagaatcgcatgagcctaggaggcagaggttgcagagtgccaagggctcaccactgcattccagcctgcccaacagagtgagacactgtttctgaaaaaaaaaaatatatatatatatatatatatgtgtgtatatatatatgtatatatatatgacttcctattaaaaactttatcccagtcgggggcagtggctcacgcctgtaatcccaacactttgggaggctgaggcaggtggatcacctgaagtccggagtttgagaccagcctggccaacatggtgaaaccccatctctactaaaaatacaaaacttaagccaggtatggtggcgggcacctgtaatcccagttacttgggaggctgaggcaggagaatcgtttaaacccaggaggtggaggttgcagtgagctgagatcgtgccattgcactctagcctgggcaacaagagtaaaactccatcttaaaggtttgtttgtttttttttaatccggaaacgaagaggcgttgggccgctattttctttttctttctttctttctttctttttttttttttctgagacggagtctagctctgctgcccaggctggagtacaatgacacgatgttggctcactgcaacctccacctcctgggttcaagcgattctcctgcctcagcctcccaagtacctgggattacaggcacctgccactacacctggcgaatatttgttttttttagtagagacgggcttttaccatgttaggctggtctcaaactcctgacctcaggtgatctgcctgccttggcctcccaaagtgctgggattacaggtgcaggccaccacacccggccttgggccactgttttcaaagtgaattgtttgttgtatcgagtccttaagtatggatatatatgtgaccctaattaagaactaccagattggatcaactaatcatgtcagcaatgtaaataactttatttttcatattcaaaataaaaactttcttttatttctggcccctttataaccagcatctttttgctttaaaaaatgacctggctttgtatttttttagtcttaaacataataaaaatatttttgttctaatttgctttcatgagtgaagattattgacatcgttggtaaattctagaattttgattttgttttttaatttgaagaaaatctttgctattattattttttccaagtggtctggcattttaagaattagtgctaataacgtaacttctaaatttgtcgtaattggcatgtttaatagcatatcaaaaaacattttaagcctgtggattcatagacaaagcaatgagaaacattagtaaaatataaatggatattcctgatgcatttaggaagctctcaattgtctcttgcatagttcaaggaatgttttctgaatttttttaatgctttttttttttttgaaagaggaaaacatacatttttaaatgtgattatctaatttttacaacactgggctattaggaataactttttaaaaattactgttctgtataaatatttgaaattcaagtacagaaaatatctgaaacaaaaagcattgttgtttggccatgatacaagtgcactgtggcagtgccgcttgctcaggacccagccctgcagcccttctgtgtgtgctccctcgttaagttcatttgctgttattacacacacaggccttcctgtctggtcgttagaaaagccgggcttccaaagcactgttgaacacaggattctgttgttagtgtggatgttcaatgagttgtattttaaatatcaaagattattaaataaagataatgtttgcttttcta

SEQ ID NO: 3 - RNA, 22 nt, Homo sapiens
gccguuggcuccagacaaauaa

SEQ ID NO: 4 - RNA, 22 nt, Homo sapiens
aauaaacauggaguccaucuuc

SEQ ID NO: 5 - RNA, 22 nt, Homo sapiens
uacagcagccuucuggaaauau

SEQ ID NO: 6 - RNA, 22 nt, Homo sapiens
cuucuggaaauauggaugacag

SEQ ID NO: 7 - RNA, 22 nt, Homo sapiens
aaguuugggguuuagaacuaau

SEQ ID NO: 8 - RNA, 22 nt, Homo sapiens
aacuaauccuguucaacagucc

SEQ ID NO: 9 - RNA, 22 nt, Homo sapiens
aacacugguuuacaguuagaaa

SEQ ID NO: 10 - RNA, 22 nt, Homo sapiens
aauuaggaaaacagugguuuaa

SEQ ID NO: 11 - RNA, 22 nt, Homo sapiens
aaguaugcaagguaguuccaga

SEQ ID NO: 12 - RNA, 22 nt, Homo sapiens
uacuucagaagagcuucggaag

SEQ ID NO: 13 - RNA, 22 nt, Homo sapiens
gagacgagaagccuacuuugaa

SEQ ID NO: 14 - RNA, 22 nt, Artificial Sequence
  1st RNA Sequence
uuucuaacuguaaaccaguguu

SEQ ID NO: 15 - RNA, 22 nt, Artificial Sequence
  1st RNA Sequence
uuaaaccacuguuuuccuaauu

SEQ ID NO: 16 - RNA, 22 nt, Artificial Sequence
  1st RNA Sequence
ucuggaacuaccuugcauacuu

SEQ ID NO: 17 - RNA, 22 nt, Artificial Sequence
  1st RNA Sequence
uucaaaguaggcuucucgucuc

SEQ ID NO: 18 - RNA, 17 nt, Artificial Sequence
  2nd RNA Sequence
cugguuuacaguuagaa

SEQ ID NO: 19 - RNA, 17 nt, Artificial Sequence
  2nd RNA Sequence
aggaaaacagugguuua

SEQ ID NO: 20 - RNA, 17 nt, Artificial Sequence
  2nd RNA Sequence
augcaagguaguuccag

SEQ ID NO: 21 - RNA, 17 nt, Artificial Sequence
  2nd RNA Sequence
cgagaagccuacuuuga

SEQ ID NO: 22 - RNA, 39 nt, Artificial Sequence
  1st - 2nd RNA Sequence
uuucuaacuguaaaccaguguucugguuuacaguuagaa

SEQ ID NO: 23 - RNA, 39 nt, Artificial Sequence
  1st - 2nd RNA Sequence
uuaaaccacuguuuuccuaauuaggaaaacagugguuua

SEQ ID NO: 24 - RNA, 39 nt, Artificial Sequence
  1st - 2nd RNA Sequence
ucuggaacuaccuugcauacuuaugcaagguaguuccag

SEQ ID NO: 25 - RNA, 39 nt, Artificial Sequence
  1st - 2nd RNA sequence
uucaaaguaggcuucucgucuccgagaagccuacuuuga

SEQ ID NO: 26 - RNA, 71 nt, Artificial Sequence
  flank - 1st - 2nd - flank RNA sequence
cuugggaauggcaagguuucuaacuguaaaccaguguucugguuuacaguuagaacucuugcuaucccaga

SEQ ID NO: 27 - RNA, 71 nt, Artificial Sequence
  flank - 1st - 2nd - flank RNA sequence
cuugggaauggcaagguuaaaccacuguuuuccuaauuaggaaaacagugguuuacucuugcuaucccaga

SEQ ID NO: 28 - RNA, 71 nt, Artificial Sequence
  flank - 1st - 2nd - flank RNA sequence
cuugggaauggcaaggucuggaacuaccuugcauacuuaugcaagguaguuccagcucuugcuaucccaga

SEQ ID NO: 29 - RNA, 71 nt, Artificial Sequence
  flank - 1st - 2nd - flank RNA sequence
cuugggaauggcaagguucaaaguaggcuucucgucuccgagaagccuacuuugacucuugcuaucccaga

SEQ ID NO: 30 - DNA, 25 bp, Artificial Sequence
  sequence derived from miR451 scaffold targeting SEQ ID NO.9
tttctaactgtaaaccagtgttctg

SEQ ID NO: 31 - DNA, 24 bp, Artificial Sequence
  sequence derived from miR451 scaffold targeting SEQ ID NO.9
tttctaactgtaaaccagtgttct

SEQ ID NO: 32 - DNA, 29 bp, Artificial Sequence
  sequence derived from miR451 scaffold targeting SEQ ID NO.9
tttctaactgtaaaccagtgttctggttt

SEQ ID NO: 33 - DNA, 28 bp, Artificial Sequence
  sequence derived from miR451 scaffold targeting SEQ ID NO.9
tttctaactgtaaaccagtgttctggtt

SEQ ID NO: 34 - DNA, 25 bp, Artificial Sequence
  sequence derived from miR451 scaffold targeting SEQ ID NO.9
tttctaactgtaaaccagtgttctt

SEQ ID NO: 35 - DNA, 22 bp, Artificial Sequence
  sequence derived from miR451 scaffold targeting SEQ ID NO.11
tctggaactaccttgcatactt

SEQ ID NO: 36 - DNA, 24 bp, Artificial Sequence
  sequence derived from miR451 scaffold targeting SEQ ID NO.11
tctggaactaccttgcatacttat

SEQ ID NO: 37 - DNA, 23 bp, Artificial Sequence
  sequence derived from miR451 scaffold targeting SEQ ID NO.11
tctggaactaccttgcatactta

SEQ ID NO: 38 - DNA, 21 bp, Artificial Sequence
  sequence derived from miR451 scaffold targeting SEQ ID NO.11
tctggaactaccttgcatact

SEQ ID NO: 39 - DNA, 30 bp, Artificial Sequence
  sequence derived from miR451 scaffold targeting SEQ ID NO.11
tctggaactaccttgcatacttatgcaagg

SEQ ID NO: 40 - DNA, 24 bp, Artificial Sequence
  sequence derived from miR451 scaffold targeting SEQ ID NO.13
ttcaaagtaggcttctcgtctccg

SEQ ID NO: 41 - DNA, 23 bp, Artificial Sequence
  sequence derived from miR451 scaffold targeting SEQ ID NO.13
ttcaaagtaggcttctcgtctcc

SEQ ID NO: 42 - DNA, 25 bp, Artificial Sequence
  sequence derived from miR451 scaffold targeting SEQ ID NO.13
ttcaaagtaggcttctcgtctccga

SEQ ID NO: 43 - DNA, 26 bp, Artificial Sequence
  sequence derived from miR451 scaffold targeting SEQ ID NO.13
ttcaaagtaggcttctcgtctccgag

SEQ ID NO: 44 - DNA, 27 bp, Artificial Sequence
  sequence derived from miR451 scaffold targeting SEQ ID NO.13
ttcaaagtaggcttctcgtctccgagt

SEQ ID NO: 45 - DNA, 21 bp, Artificial Sequence
  sequence derived from miR451 scaffold targeting SEQ ID NO.13
ttcaaagtaggcttctcgtct

SEQ ID NO: 46 - DNA, 22 bp, Artificial Sequence
  sequence derived from miR451 scaffold targeting SEQ ID NO.13
ttcaaagtaggcttctcgtctc

SEQ ID NO: 47 - DNA, 27 bp, Artificial Sequence
  sequence derived from miR451 scaffold targeting SEQ ID NO.13
ttcaaagtaggcttctcgtctccgaga

SEQ ID NO: 48 - DNA, 25 bp, Artificial Sequence
  sequence derived from miR451 scaffold targeting SEQ ID NO.13
ttcaaagtaggcttctcgtctccgt

SEQ ID NO: 49 - DNA, 2440 bp, Artificial Sequence
  expression cassette sequence
cagtcacgacgttgtaaaacgacggccagtgaattcgcccttcaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgcttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaagggcgaattcgagctcggtacctcgcgaatgcatctagatatcggcgctatgcttcctgtgcccccagtggggccctggctgggatttcatcatatactgtaagtttgcgatgagacactacagtatagatgatgtactagtccgggcacccccagctctggagcctgacaaggaggacaggagagatgctgcaagcccaagaagctctctgctcagcctgtcacaacctactgactgccagggcacttgggaatggcaaggtctggaactaccttgcatacttatgcaaggtagttccagtcttgctatacccagaaaacgtgccaggaagagaactcaggaccctgaagcagactactggaagggagactccagctcaaacaaggcaggggtgggggcgtgggattgggggtaggggagggaatagatacattttctctttcctgttgtaaagaaataaagataagccaggcacagtggctcacgcctgtaatcccaccactttcagaggccaaggcgctggatccagatctcgagcggccgcccgtggcatccctgtgacccctccccagtgcctctcctggccctggaagttgccactccagtgcccaccagccttgtcctaataaaattaagttgcatcaagatcgacgggcccgtcgactgcagag

SEQ ID NO: 50 - RNA, 16 nt, Artificial Sequence
  5′ flanking sequence
cuugggaauggcaagg

SEQ ID NO: 51 - RNA, 17 nt, Artificial Sequence
  3′ flanking sequence
cucuugcuauacccaga

SEQ ID NO: 52 - RNA, 71 nt, Artificial Sequence
  Pre-miC_451
  misc_feature (17)..(55): n is a, c, g, or u
cuugggaauggcaaggnnnnnnnnnnnnnnnnnnnnnnnnnnnnnnnnnnnnnnnucuugcuauacccaga

(19) United States
(12) Patent Application Publication
(10) Pub. No.: US 2020/0199625 A1
(43) Pub. Date: Jun. 25, 2020

(54) RNAi induced reduction of ataxin-3 for the treatment of Spinocerebellar ataxia type 3

(71) Applicant: uniQure IP B.V., Amsterdam (NL)

(72) Inventors: Melvin Maurice EVERS, Amsterdam (NL); Pavlina Stefanova KONSTANTINOVA, Amsterdam (NL); Raygene Michael MARTIER, Amsterdam (NL)

(73) Assignee: uniQure IP B.V., Amsterdam (NL)

(21) Appl. No.: 16/684,028

(22) Filed: Nov. 14, 2019

Related U.S. Application Data
(60) Provisional application No. 62/769,092, filed on Nov. 19, 2018.

(30) Foreign Application Priority Data
Nov. 19, 2018 (EP) 18206963.3
May 1, 2019 (EP) 19172083.8

(51) Int. Cl. C12N 15/86; C12N 15/113; A61K 48/00; C12N 7/00; A61P 25/28

(52) U.S. Cl. CPC C12N 15/86; C12N 15/113; A61K 48/0066; C12N 2750/14143; A61P 25/28; C12N 2320/30; C12N 7/00

(57) ABSTRACT

The current invention relates to gene therapy approaches for the treatment of SCA3, in particular RNAi based gene therapy approaches utilizing a total knockdown approach. The inventors provide for selected target regions and/or target sequences for which highly efficient knockdown of the ATXN3 gene expression can be advantageously obtained in human neuronal cells and in mouse models relevant for SCA3.

### CROSS-REFERENCE

This application claims the benefit of U.S. Patent Application No. 62/769,092 filed Nov. 19, 2018. This application also claims the benefit of EP Application No. 19172083.8 filed May 1, 2019 and EP Application No. 18206963.3 filed Nov. 19, 2018. All the foregoing are incorporated by reference in their entireties.

### INCORPORATION OF SEQUENCE LISTING

The material in the accompanying sequence listing is hereby incorporated by reference into this application. The accompanying sequence listing text file, nameISALFSQL.txt, was created on Nov. 14, 2019, and is 117 kb. The file can be assessed using Microsoft Word on a computer that uses Windows OS.

### BACKGROUND

Spinocerebellar ataxia type 3 (SCA3), or Machado-Joseph disease (MJD), is an autosomal dominant monogenic, fatal disorder. The disorder is characterized by progressive degeneration of brain areas, which is caused by a CAG expansion in the human ataxin-3 gene, also referred to as ATXN3 gene (OMIM: 607047, reference sequence Homo sapiens ataxin 3 (ATXN3) on chromosome 14, NCBI Reference Sequence: NG_008198.2 (SEQ ID NO.1). As depicted in FIG. 1, in the 3′ region of the gene and gene transcript, in an exon sequence, which exon sequence corresponds with the sequence corresponding with nts 942-1060 of SEQ ID NO.2, (i.e. in most ATXN3 transcript variants corresponding with exon 10 as depicted in FIG. 1), a cytosine-adenine-guanine (CAG) repeat region is present interspersed with no, one or two CAA codons (corresponding with nts. 943-984 of SEQ ID NO.2 as depicted in FIG. 1). Said CAG region is in frame and results in an ataxin-3 protein comprising a polyQ region, a repetitive sequence of glutamines. The CAG repeat region as depicted in FIG. 1 represents a region that is not associated with disease. Healthy, or non-symptomatic, individuals may have up to 44 CAG-repeats in the ATXN3 gene. Diseased individuals have expansions and it has been shown that they may have between 52 and 86 or more CAG repeats. Individuals having between 45-51 CAG repeats are to have symptoms with incomplete penetrance of disease. Said expansion resulting in ataxin-3 protein that have extended polyQ regions and the length of the CAG repeats, and thus polyQ regions within ataxin-3, can be correlated with disease progression, i.e. the longer the region usually the more progressive the disease.

The ataxin-3 protein with the expanded polyQ tract acquires toxic properties (gain of toxic function) and the formation of neuronal aggregates in the brain is the neuropathological hallmark. Neuropathological studies have detected widespread neuronal loss in various areas, including cerebellum, thalamus, midbrain, pons, medulla oblongata and spinal cord of SCA3 patients (Riess et al., Cerebellum 2008). Although widespread pathology is reported, the consensus is that the main pathology is in the cerebellum and in the brainstem (Eichler et al. AJNM Am J Neuroradiol, 2011). The disease has full penetration, which means that if a person has an expansion of 52 or more CAGs, they will inevitably develop the disease and have 50% chance to pass it on to their offspring.

RNA interference (RNAi) is a naturally occurring mechanism that involves sequence specific down regulation of messenger RNA (mRNA). The down regulation of mRNA results in a reduction of the amount of protein that is expressed. RNA interference is triggered by double stranded RNA. One of the strands of the double stranded RNA is substantially or completely complementary to its target, the mRNA. This strand is termed the guide strand. The mechanism of RNA interference involves the incorporation of the guide strand in the RNA-induced silencing complex (RISC). This complex is a multiple turnover complex that via complementary base paring binds to its target mRNA. Once bound to its target mRNA it can either cleave the mRNA or reduce translation efficiency. RNA interference has since its discovery been widely used to knock down specific target genes and thereof lowering of the subsequent protein expression. Methods for inducing RNA interference involve the use of small interfering RNA (siRNA), and/or short hairpin RNA (shRNA). In addition, molecules that can naturally trigger RNAi, the so-called miRNAs, have been used to make artificial miRNAs that mimic their naturally occurring counterparts. These strategies have in common that they provide for substantially double stranded RNA molecules that are designed to target a gene of choice. RNAi based therapeutic approaches that utilise the sequence specific modality of RNAi are under development and several are currently in clinical trials.

RNAi gene therapy approaches have been proposed as treatment for SCA3. The focus of such approaches has been mainly to selectively knock-down human ATXN3 transcripts with expanded repeats (Alves, et al., Plos One, Vol. 3 Iss. 10, 2008; Fiszer et al., BMC Mol Biol. 13:6, 2012; WO2006031267; and Rodriguez-Lebron et al. Mol Ther., vol. 21, no. 10, 2013). This selective knock-down involves the targeting of a SNP in disease associated transcripts not found in genes associated with healthy, i.e. non-SCA3 diseased, humans. Despite the demonstrated effective suppression of ATXN3 in the cerebellum and safety of the knockdown approach used, when looking at the motor phenotype and survival it was observed that motor impairment was not ameliorated and survival not prolonged (Costa et al., Mol Ther, vol. 21, no. 10, 2013). There is thus a need for improved RNAi gene therapy approaches as a treatment for SCA3.

### SUMMARY OF THE INVENTION

The present invention provides for a novel RNAi approach aimed at obtaining knock-down of both disease and non-disease associated ATXN3 transcripts (OMIM: 607047) rather than being aimed at selectively targeting transcripts associated with disease. In particular, highly efficient knock-down of disease and non-disease associated ATXN3 transcripts could be obtained by targeting sequences 5′ from the CAG repeat. Preferably, the sequence targeted is found in the region corresponding with nucleotides 390-941 of SEQ ID NO.2. SEQ ID NO. 2 is depicted in FIG. 1. In the sequence depicted in FIG. 1, this preferred target sequence corresponds with exons 5, 6, 7, 8, and 9. It is understood that the ATXN3 transcripts can be composed of different exons, and thus the order of exons may be different as is depicted in FIG. 1 (Bettencourt et al., Neurogenetics, 2010). Sequences corresponding with exons 5, 6, 7, 8 and 9, as depicted in FIG. 1 and corresponding respectively with nucleotides 390-456, 457-544, 545-677, 678-844 and 845-941 of SEQ ID NO.2 are comprised in ATXN3 transcripts. As ATXN3 transcript variants may have different exon compositions, targeting sequences representing different exon compositions is also encompassed by the present invention, as long as the target sequence is comprised in about the 550 nucleotides found directly 3′ from the CAG repeat of spliced ATXN3 transcripts, such a target sequence may be contemplated in accordance with the invention. Also, as ATXN3 transcript variants may have different exon compositions, targeting sequences representing different exon compositions is also encompassed by the present invention, as long as the target sequence is comprised in at least one of the sequences corresponding with exons 5, 6, 7, 8 and 9, as depicted in FIG. 1 and corresponding respectively with nucleotides 390-456, 457-544, 545-677, 678-844 and 845-941 of SEQ ID NO.2, such a target sequence may be contemplated in accordance with the invention. This is because, as shown in the examples, in the regions 5′ from the CAG repeat highly efficacious knock down of ATXN3 gene expression could be achieved, despite the large number of alternative splice variants generated in this region. By reducing both disease and non-disease associated transcripts and/or targeting 5′ from the CAG repeat, highly efficient lowering of the ataxin-3 protein could be obtained. Targeting 5′ from the CAG repeat region also allowed to obtain most efficient knockdown of ataxin-3 containing the expanded polyQ as most naturally occurring splice variants are targeted.

### BRIEF DESCRIPTION OF THE DRAWINGS

FIG. 1 discloses a schematic of part of the ATXN3 cDNA sequence comprising the CAG repeat region (comprised in exon 10), and with selected target RNA sequences indicated (SEQ ID NOs. 3-13). The sequence listed is part of NCBI Reference Sequence: NM_004993.5, the sequence depicted is referred to as SEQ ID NO.2 herein, and corresponds to nucleotides 1-1329 thereof, and represents DNA sequence (cDNA) of (part of) a spliced ATXN3 transcript. Hence, the corresponding RNA, has the same sequence except having instead of a T a U as depicted in FIG. 1 and SEQ ID NO.2, Nucleotides 1-93 represent exon 1, nucleotides 94-258 represent exon 2, nucleotides 259-303 represent exon 3, and nucleotides 304-389 represent exon 4. Nucleotides 390-941 encompasses exons 5, 6, 7, 8 and 9. Exons 5, 6, 7, 8, and 9 are represented respectively by 390-456, 457-544, 545-677, 678-844 and 845-941. The exon 10 sequence corresponds with 942-1060 of SEQ ID NO.2 and comprises a repeat region of 14 codons comprising 12 CAGs. The selected target RNA sequences (as listed in table 1) i.e. the DNA sequence corresponding thereto, are depicted in FIG. 1 as well. SEQ ID NO.3 corresponds with nucleotides 46-67, in exon 1; SEQ ID NO.4 corresponds with nucleotides 63-84, in exon 1; SEQ ID NO.5 corresponds with nucleotides 254-275, in exon 2-3; SEQ ID NO.6 corresponds with nucleotides 263-284, in exon 3; SEQ ID NO.7 corresponds with nucleotides 323-244, in exon 4; SEQ ID NO.8 corresponds with nucleotides 338-359, in exon 4; SEQ ID NO.9, corresponds with nucleotides 422-443, in exon 5; SEQ ID NO.10 corresponds with nucleotides 443-464, in exon 5-6; SEQ ID NO.11, corresponds with nucleotides 834-855, in exon 8-9; SEQ ID NO.12 corresponds with nucleotides 897-918, in exon 9; SEQ ID NO.13 corresponds with nucleotides 918-939, in exon 9.

FIG. 2A discloses a schematic of miR451 scaffold RNA structure indicating the first RNA sequence as it is designed. FIG. 2B discloses a schematic of expression cassette of a miRNA scaffold. FIG. 2C discloses a schematic showing Renilla/Firefly construct, with Renilla construct comprising an inserted target sequence (black box).

FIG. 3 discloses a graph showing silencing of ATXN3 reporters by targeting SEQ ID NOs. 3-13. HEK293T cells were co-transfected in a 1:0.1 to 100 ratio with the luciferase reporter constructs and the different scaffolds targeting SEQ ID NOs. 3-1. Renilla and firefly were measured 2 days post-transfection and Renilla was normalized to firefly expression. Scrambled miRNA (CTRL) served as a negative control and was set at 100% (y-axis). Targeting SEQ ID NOs 9-13 resulted in most strong knockdown, achieving about 75% or more knockdown at the highest level, with SEQ ID NO.11 targeting showing the most prominent knockdown (>90%).

FIG. 4A discloses a western Blot showing lowering of endogenous ataxin-3 protein in 293T cells. FIG. 4B discloses the quantification of the western blot.

FIG. 5A discloses a graph showing dose response of AAV-miRNA transduction in iPSC-derived neurons. Increased dosages ranging from 10exp 10, 10exp 11 and 10exp 12 were used to transduce neurons. Mature miRNA was determined in the neurons and a dose response curve was shown, with the higher dose showing the highest level of expression. FIG. 5B discloses this dose response curve resulted in a dose response curve having the reverse image when determining ATXN3 mRNA levels, the higher the dose of AAV, the lower the amount of ATXN3 mRNA levels detected. The lowest amount of ATXN3 mRNA was detected when targeting SEQ ID NO.11.

FIG. 6A discloses the in vivo administration of AAV targeting SEQ ID NO. 9, 11 and 13. AAV was injected in the mouse. The amount of gc detected per genomic DNA was determined for each administration and each area. The gc detected per area were similar per injection site, with variation between injection sites. FIG. 6B discloses the knockdown of ATXN3 mRNA was determined in the medulla. All three target regions showed similar reduction in mRNA.

FIG. 7 discloses the DNA sequence of an expression construct (SEQ ID NO. 49) encoding a miR451 scaffold comprising a first RNA sequence of 22 nucleotides targeting SEQ ID NO.11. The expression cassette comprises a CAG promoter shown in bold (position 43-1712), the sequence encoding the first RNA sequence shown in bold and underlined (position 2031-2052, encoding SEQ ID NO. 16), the sequence encoding the second RNA sequence is shown underlined (position 2053-2070, encoding SEQ ID NO. 20), the hGH poly A signal shown in bold and italics (2318-2414). The pri-miRNA sequence comprises a pre-miRNA sequence. The pri-miRNA encoding sequence is shown between [brackets](position 2015-2086, encoding SEQ ID NO. 28). The pre-miRNA sequence comprises the first RNA sequence and the second RNA sequence and the sequence encoding it is shown underlined, either normal or bold, (position 2031-2070) (encoding SEQ ID NO. 24). The pre-miRNA or pri-miRNA encoding sequence may be replaced e.g. by a sequence encoding a pre-miRNA or pri-miRNA as listed in tables 4 and 5, respectively and as depicted in FIG. 8. The first RNA sequence of the pre-miRNA or pri-miRNA can be any sequence of 22 nucleotides selected to bind and target a sequence in the ATXN3 gene, preferably a target nucleotide sequence 5′ from the CAG repeat region of the ATXN3 gene and such as listed e.g. in table 1. The second RNA sequence is selected and adapted to be complementary to the first RNA sequence. The secondary structure is checked on mfold by folding the RNA sequence using standard settings utilizing the RNA folding form, with folding temperature fixed at 37 degrees Celcius (as available online <unafold.rna.albany.edu/?q=mfold>; Zuker et al., Nucleic Acids Res. 31 (13), 3406-15, (2003)) for folding, and adapted if necessary, into a miR-451 pri-miRNA structure as depicted in FIGS. 2a and 8.

FIGS. 8A-8H disclose predicted RNA structures of selected pre-miRNA (FIG. 8A-8D) and pri-miRNA (FIGS. 8E-8H) sequences in an miR451 scaffold. Sequences A and E, B and F, C and G, D and H are predicted pre-miRNA and pri-miRNA structures targeting the respective target sequences SEQ ID NO. 9, 10, 11 and 13. Sequences of the secondary RNA sequences depicted are listed in Tables 4 and 5. Structures were made using M-fold using standard settings, utilizing the RNA folding form, (as available online <unafold.rna.albany.edu/?q=mfold>; Zuker et al., Nucleic Acids Res. 31 (13), 3406-15, (2003). Standard settings used for m-fold version 3.5 were as follows: RNA sequence is linear, folding temperature is fixed at 37°, ionic conditions: 1M NaCl, no divalent ions, percent suboptimality number is 5, interior/bulge loop size is 30, maximum asymmetry of an interior/bulge loop is 30, and no maximum distance between paired bases. FIG. 8A discloses the predicted RNA structure of pre-miRNA targeting target sequence SEQ ID NO. 9. FIG. 8B discloses the predicted RNA structure of pre-miRNA targeting target sequence SEQ ID NO. 10. FIG. 8C discloses the predicted RNA structure of pre-miRNA targeting target sequence SEQ ID NO. 11. FIG. 8D discloses the predicted RNA structure of pre-miRNA targeting target sequence SEQ ID NO. 13. FIG. 8E discloses the predicted RNA structure of pri-miRNA targeting target sequence SEQ ID NO. 9. FIG. 8F discloses the predicted RNA structure of pri-miRNA targeting target sequence SEQ ID NO. 10. FIG. 8G discloses the predicted RNA structure of pri-miRNA targeting target sequence SEQ ID NO. 11. FIG. 8H discloses the predicted RNA structure of pri-miRNA targeting target sequence SEQ ID NO. 13.

FIGS. 9A-9D disclose vector copy distribution of AAV5 in F512 SCA3 mice. FIG. 9A discloses schematic representation of the routes of administration. Three months old mice (N-3) were injected ICV, or in the cisterna magna, or DCN with AAV5-miATXN3_9, AAV5-miATXN3_11 or AAV5-miATXN3_13. 10 μl of AAV5 were injected either ICV or in the cisterna magna and 2 μl were injected bilaterally in the DCN. The injection sites are depicted in dark grey, indicated with arrow. All mice were sacrificed 6 weeks after surgeries. FIG. 9B discloses vector copy distribution in cortex, cerebellum and brain stem after ICV injection. FIG. 9C discloses vector copy distribution in cortex, cerebellum and brain stem after cisterna magna injection. FIG. 9D discloses vector copy distribution in cortex, cerebellum and brain stem after DCN injection. DNA was isolated from the cortex, cerebellum and brain stem tissues and qPCR was performed to determine the vector copy distribution. The genomic copies per μg DNA was calculated for each brain region using a standard curve.

FIGS. 10A-10H disclose silencing of mutant ataxin-3 in F512 mice. FIG. 10A discloses expression of mature miATXN3 guide strands in the cerebellum after DCN administration. Total RNA was isolated from the cerebellum for small RNA TaqMan. MicroRNA input levels were normalized to U6 small nuclear RNA and set relative to AAV-GFP treated mice. FIG. 10B discloses lowering of total ATXN3 mRNA in cerebellum of DCN injected mice. Total RNA was isolated from cerebellum and RT-qPCR was performed to detect the mouse wildtype ATXN3 mRNA. RNA input levels were normalized to GAPDH and set relative to AAV-GFP treated mice. FIG. 10C discloses expression of mature miATXN3 guide strands in the brain stem after cisterna magna administration. Performed as described for FIG. 10A. FIG. 10D discloses lowering of total ATXN3 mRNA in cerebellum of cisterna magna injected mice. Performed as described for FIG. 10B. FIG. 10E discloses expression of mature miATXN3 guide strands in the brain stem after cisterna magna administration. Performed as described for FIG. 10A. FIG. 10F discloses lowering of total ATXN3 mRNA in brain stem of cisterna magna injected mice. Performed as described for FIG. 10B. FIG. 10G discloses reduction of mutant ataxin-3 protein in the brain stem after cisterna magna delivery. TR-FRET immunoassay was performed on tissue lysates to specifically detect the mutant ataxin-3 (no detection of wildtype mouse ataxin-3). The protein expression is shown in percentage relative to the control (untreated) mice. Strong lowering of mutant ataxin-3 protein in the brainstem up to 64.5% was observed. FIG. 10H discloses reduction of mutant ataxin-3 protein in the cerebellum after cisterna magna delivery. A robust ataxin-3 protein lowering of 53.1% in the cerebellum was observed.

FIGS. 11A-11D disclose miATXN3 mediated ataxin-3 knockdown in SCA3 mouse brain. Mice were stereotaxically injected at 2 months of age with a mixture of a mutant ataxin-3 lentiviral expression cassette and AAV5-miATXN3_11 in both striata. The lentiviral construct results in expression of mutant ataxin-3 throughout the striatum during the study period. FIG. 11A discloses bodyweight of mice was not negatively affected by any of the tested doses of miATXN3. FIG. 11B discloses qPCR analysis revealed a strong dose dependent knockdown of mutant ATXN3 expression in the striatum 7 weeks after AAV5-miATXN3 treatment. FIG. 11C discloses soluble ataxin-3 protein levels were reduced up to 90% in the striatum after high dose of miATXN3 as quantified through western blot analysis. FIG. 11D discloses the insoluble and aggregated ataxin-3 protein fraction in striatum was almost completely abolished by mid and high dose treatment of miATXN3. LD=low dose (2×10⁹ gc), MD=mid dose (1×10¹⁰ gc) HD=high dose (2×10¹⁰ gc) PBS n=8, AAV5 HD n=8; AAV5 MD n=8; AAV5 LD n=8. one-way ANOVA (*p<0.05, **p<0.01 ***p<0.001 and ****p<0.0001).

FIGS. 12A-12D discloses reduction in ataxin-3 inclusions and darpp32 lesion size in SCA3 mice. Striatum from right hemisphere of miATXN3 treated SCA3 mice were stained for ataxin-3 and darpp-32 (dopamine- and cAMP-regulated neuronal phosphoprotein). FIG. 12A discloses ataxin-3 stained (1H9) striatum of mice sacrificed 7 weeks after miATXN3 treatment shows presence of nuclear inclusions in PBS treated SCA3 mice. FIG. 12B discloses right hemisphere of mice was stained with the midbrain dopaminergic neuron marker darpp-32. A darpp-32 depleted lesion representing the early neuronal dysfunction can be seen in the PBS treated animals close to the injection site (top left of striatum). FIG. 12C discloses quantification of nuclear ataxin-3 inclusions in striatum. Low dose miATXN3 treatment significantly reduced the number of ataxin-3 inclusions by about 50%. Presence of nuclear ataxin-3 inclusions was almost completely abolished in mid and high dose miATXN3 treated animals. FIG. 12D discloses quantification of darpp-32 depleted volume. Total lesion size was calculated for the whole striatum based on interspaced sections. Lesion size was significantly reduced in a dose dependent matter following miATXN3 treatment compared to PBS treated animals, indicating a reduction in neuronal dysfunction. PBS n=8, AAV5 HD n=8; AAV5 MD n=7; AAV5 LD n=8. one-way ANOVA (*p<0.05, **p<0.01 ***p<0.001 and ****p<0.0001)

FIGS. 13A-13B disclose effect of miATXN3 treatment on endogenous mouse ataxin-3 protein levels. Based on qPCR and western blot data from FIG. 11. FIG. 13A discloses quantification of murine ataxin-3 RNA. FIG. 13B discloses quantification of murine ataxin-3 protein. Only minor downregulation of endogenous ataxin-3 at the high dose of AAV5-miATXN3 (2×10⁹) in the mouse striatum observed. Endogenous mouse ataxin-3 carries a one nucleotide mismatch in the target sequence. ** p<0.01, one-way ANOVA.

FIG. 14 discloses effect of intrathecal administration of miATXN3 on endogenous non-humane primate ataxin-3 protein levels. Quantification of Macaca fascicularis ataxin-3 protein shows downregulation of endogenous ataxin-3 in the non-human primate brain. Time-resolved fluorescence energy transfer (TR-FRET) was used to quantify ataxin-3 protein and expression levels calculated relative to the mean of control microRNA (miCRTL)-treated samples. Brain punches analysed were; p26 motor cortex; p32 putamen; p69, p70, p71 pons; p72 occipital cortex, p78 deep cerebellar nucleus; p89 and p91 cerebellar cortex. N=3 per treatment.

### DETAILED DESCRIPTION

The current invention relates to gene therapy, and in particular to the use of RNA interference in gene therapy for targeting RNA encoded by the human ATXN3 gene (OMIM: 607047). Expanded CAG repeats, (CAGn), in the ATXN3 gene are associated with Spinocerebellar ataxia type 3 (SCA3), also referred to as Machado-Joseph disease (MJD), which is an autosomal dominant monogenic, fatal disorder. Hence, reducing RNA expression levels is aimed to reduce the neuropathology associated with RNAs containing expanded CAG repeats and/or of ataxin-3 protein containing expanded polyQ translated therefrom. Combined targeting of the brain stem and the cerebellum using a gene therapy approach as outlined herein is to thereby significantly benefit affected human patients by slowing down or complete halting of further neuropathologies.

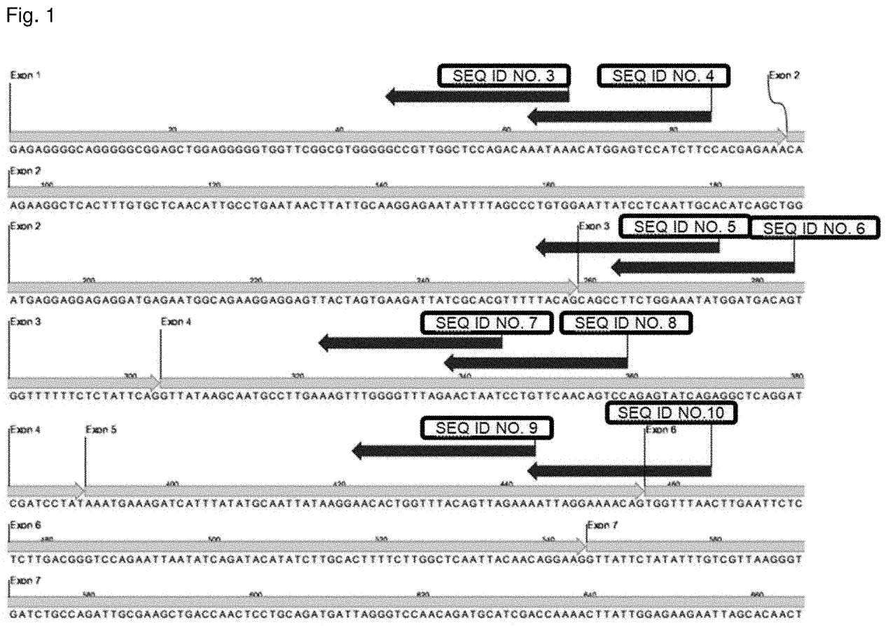

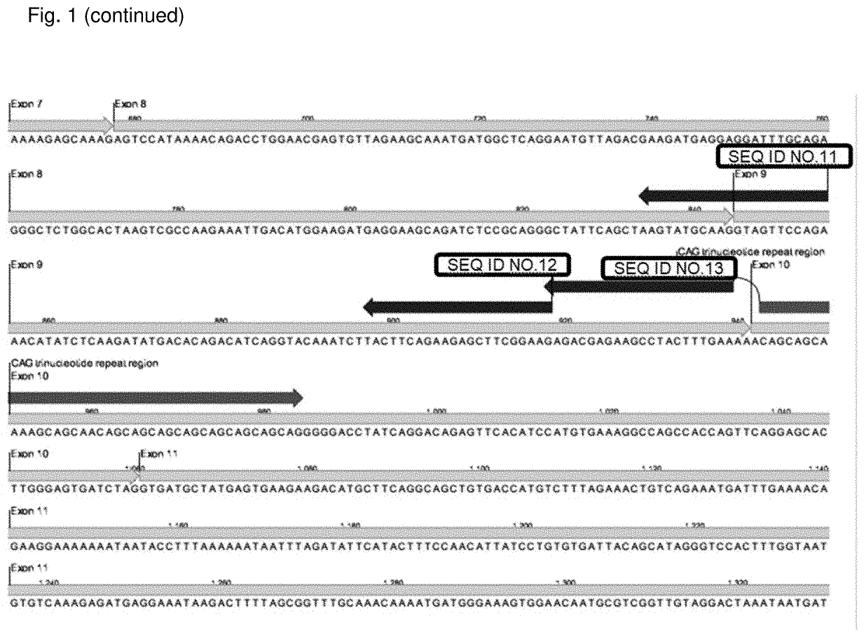

Hence, the current invention now provides for an expression cassette encoding a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human ATXN3 gene (OMIM: 607047). In particular, it has been found useful to target a sequence of the human ATXN3 gene that is 5′ to the CAG repeat as shown in SEQ ID NO.2 and e.g. as shown in FIG. 1. By targeting human ATXN3 this way, the current inventors were able to highly efficiently reduce human ATXN3 gene expression and thus to reduce formation of ataxin-3 protein. Ultimately this may halt and/or stop further neuropathologies.

The first RNA sequence that is to be expressed in accordance with the invention is to be comprised, in whole or a substantial part thereof, in a guide strand, also referred to as antisense strand as it is complementary (“anti”) to a sense target RNA sequence, the sense target RNA sequence being comprised in an RNA encoded by a human ATXN3 gene. The second RNA sequence, which is also referred to as “sense strand”, may have substantial sequence identity with, or be identical to, the target RNA sequence. The first and second RNA sequences are comprised in a double stranded RNA and are substantially complementary. Said double stranded RNA according to the invention is to induce RNA interference, thereby reducing expression of ATXN3 transcripts, which includes knocking down of CAG repeat containing transcripts, knocking down expression of both disease associated expanded CAG repeat containing transcripts and non-disease associated CAG repeat containing ATXN3 transcripts. Transcripts that may be targeted may include spliced, including splice variants, and unspliced RNA transcripts such as encoded by SEQ ID NO.1. Hence, an RNA encoded by a human ATXN3 gene is understood to comprise unspliced mRNAs comprising a 5′ untranslated region (UTR), intron and exon sequences, followed by a 3′ UTR and a polyA tail, and also splice variants thereof. Said double stranded RNA according to the invention may also induce transcriptional silencing. It is understood that in accordance with the invention, instead of providing an expression cassette, a first and second RNA sequence as described herein may be provided, said first and second RNA sequence targeting an RNA encoded by a human ATXN3 gene.

It is understood that ‘substantially complementary’ in this context means that it is not required to have all the nucleotides of the first and second RNA sequences to be base paired, i.e. to be fully complementary, or all the nucleotides of the first RNA sequence and the target RNA sequence to be base paired. As long as the double stranded RNA is capable of inducing RNA interference to thereby sequence-specifically target a sequence comprising the target RNA sequence, such substantial complementarity is contemplated in accordance with the invention.

In one embodiment the double stranded RNA according to the invention comprises a first RNA sequence and a second RNA sequence, wherein the first and second RNA sequence are substantially complementary, and wherein the first RNA sequence has a sequence length of at least 19 nucleotides and is substantially complementary to a target RNA sequence of an RNA encoded by a human ATXN3 gene, which first RNA sequence is capable of inducing RNA interference to sequence-specifically reduce expression of an RNA transcript comprising the target RNA sequence. In a further embodiment, said induction of RNA interference to reduce expression of an RNA transcript comprising the target RNA sequence means that it is to reduce human ATXN3 gene expression. It is understood that wherein the terms ‘RNA sequence’, ‘(m)RNA’, ‘RNA strand’, or ‘RNA molecule’ are used herein that these terms refer to the same physical entity, i.e. a (bio)polymer consisting of nucleotide monomers covalently bonded in a chain. The term ‘double stranded RNA’ may also refer to such a physical entity, which may correspond with two chains consisting of nucleotide monomers covalently bonded, or may correspond with one chain, e.g. two chains covalently connected via nucleotide monomers covalently bonded that form a loop sequence such as in a shRNA.

One can easily determine whether reduced expression of an RNA transcript comprising the target RNA sequences is indeed the case by using e.g. standard luciferase reporter assays and appropriate controls such as described in the examples and as known in the art (e.g. Zhuang et al. 2006 Methods Mol Biol. 2006; 342:181-7). For example, a luciferase reporter comprising a target RNA sequence can be used to show that the double stranded RNA according to the invention is capable of sequence-specific knock down. Furthermore, such as shown i.a. in the example section, knock down of ataxin-3 protein expression and/or ATXN3 mRNA can be easily measured in in vitro neuronal cultures and in brain tissue obtained from (transgenic) animal models.

The double stranded RNA according to the invention is capable of inducing RNA interference (RNAi). Double stranded RNA structures that are suitable for inducing RNAi are known in the art. For example, a small interfering RNA (siRNA) can induce RNAi. An siRNA comprises two separate RNA strands, one strand comprising the first RNA sequence and the other strand comprising the second RNA sequence. An siRNA design that is often used involves 19 consecutive base pairs with a 3′ overhang. The first and/or second RNA sequence may comprise a 3′-overhang. The 3′-overhang preferably is a dinucleotide overhang on both strands of the siRNA. Such a design is based on observed endoribonuclease Dicer processing of larger double stranded RNAs as known in the art that results in siRNAs having these features. The 3′-overhang may be comprised in the first RNA sequence. The 3′-overhang may be in addition to the first RNA sequence. The length of the two strands of which an siRNA is composed may be 19, 20, 21, 22, 23, 24, 25, 26 or 27 nucleotides or more. The strand comprising the first RNA sequence may also consist of the first RNA sequence. The strand comprising the first RNA sequence may also consist of the first RNA sequence and the overhang sequence.

siRNAs may also serve as Dicer substrates. For example, a Dicer substrate may be a 27-mer consisting of two strands of RNA that have 27 consecutive base pairs. The first RNA sequence is positioned at the 3′-end of the 27-mer duplex. At the 3′-ends, like with siRNAs, each or one of the strands of the Dicer substrate may comprise a two-nucleotide overhang. The 3′-overhang may be comprised in the first RNA sequence. The 3′-overhang may be in addition to the first RNA sequence. 5′ from the first RNA sequence, additional sequences may be included that are either complementary to the sequence adjacent to the target RNA sequence, thereby extending the sequence length which is complementary to the target sequence, or not. The other end of the siRNA Dicer substrate is blunt ended. This Dicer substrate design may result in a preference in processing by Dicer such that an siRNA can be formed like the siRNA design as described above, having 19 consecutive base pairs and 2 nucleotide overhangs at both 3′-ends. In any case, siRNAs, or the like, are composed of two separate RNA strands (Fire et al. 1998, Nature. 1998 Feb. 19; 391 (6669):806-1 1) each RNA strand comprising or consisting of the first or second RNA sequence.

The first and second RNA sequences can also be comprised in an shRNA. An shRNA may comprise or consist of from the 5′-end till the 3′-end the following sequences: 5′-second RNA sequence-loop sequence-first RNA sequence-optional 2 nt overhang sequence-3′. Alternatively, a shRNA may comprise from the 5′-end till the 3′-end the following sequences: 5′-first RNA sequence-loop sequence-second RNA sequence-optional 2 nt overhang sequence-3′. Such an RNA molecule forms intramolecular base pairs via the substantially complementary first and second RNA sequence. Suitable loop sequences are well known in the art (i.a. as shown in Dallas et al. 2012 Nucleic Acids Res. 2012 October; 40(18):9255-71 and Schopman et al., Antiviral Res. 2010 May; 86(2):204-11). The loop sequence may also be a stem-loop sequence, whereby the double stranded region of the shRNA is extended. Like the siRNA Dicer substrate as described above, an shRNA can be processed by e.g. Dicer to provide for an siRNA having an siRNA design such as described above, having e.g. 19 consecutive base pairs and 2 nucleotide overhangs at both 3′-ends. In case the shRNA is to be processed by Dicer, it is preferred to have the first and second RNA sequence at the end of the shRNA, i.e. such that the putative strands of the siRNA are linked via a stem loop sequence, i.e.: 5′-first RNA sequence-stem loop sequence-second RNA sequence-optional 2 nt overhang sequence-3′. Or, conversely, 5′-second RNA sequence-stem loop sequence-first RNA sequence-optional 2 nt overhang sequence-3′. Another shRNA design may be an shRNA structure that is processed by the RNAi machinery to provide for an activated RISC complex that does not require Dicer processing (Liu et al., Nucleic Acids Res. 2013, Apr. 1; 41(6):3723-33 and Herrera-Carrillo and Berkhout, NAR, 2017, Vol. 45 No. 18 10369-79, both incorporated herein by reference), so called AgoshRNAs, which are based on a structure very similar to the miR451 scaffold as described below. Such an shRNA structure comprises in its loop sequence part of the first RNA sequence. Such an shRNA structure may also consist of the first RNA sequence, followed immediately by the second RNA sequence.

A double stranded RNA according to the invention may also be incorporated in a pre-miRNA or pri-miRNA scaffold. MicroRNAs, i.e. miRNA, are guide strands that originate from double stranded RNA molecules that are endogenously expressed e.g. in mammalian cells. A miRNA is processed from a pre-miRNA precursor molecule, similar to the processing of an shRNA or an extended siRNA as described above, by the RNAi machinery and incorporated in an activated RNA-induced silencing complex (RISC) (Tijsterman M, Plasterk R H. Dicers at RISC; the mechanism of RNAi. Cell. 2004 Apr. 2; 1 17(1):1-3). A pre-miRNA is a hairpin RNA molecule that can be part of a larger RNA molecule (pri-miRNA), e.g. comprised in an intron, which is first processed by Drosha to form a pre-miRNA hairpin molecule. The pre-miRNA molecule is an shRNA-like molecule that can subsequently be processed by Dicer to result in an siRNA-like double stranded RNA duplex. The miRNA, i.e. the guide strand, that is part of the double stranded RNA duplex is subsequently incorporated in RISC. An RNA molecule such as present in nature, i.e. a pri-miRNA, a pre-miRNA or a miRNA duplex, may be used as a scaffold for producing an artificial miRNA that specifically targets a gene of choice. Based on the predicted RNA structure of the RNA molecule as present in nature, e.g. as predicted using e.g. m-fold software using standard settings (Zuker. Nucleic Acids Res. 31 (13), 3406-3415, 2003), the natural miRNA sequence as it is present in the RNA structure (i.e. duplex, pre-miRNA or pri-miRNA), and the sequence present in the structure that is substantially complementary therewith are removed and replaced with a first RNA sequence and a second RNA sequence according to the invention. The first RNA sequence and the second RNA sequence are preferably selected such that the predicted secondary RNA structures that are formed, i.e. of the pre-miRNA, pri-miRNA and/or miRNA duplex, resemble the corresponding predicted original secondary structure of the natural RNA sequences. pre-miRNA, pri-miRNA and miRNA duplexes (that consist of two separate RNA strands that are hybridized via complementary base pairing) as found in nature often are not fully base paired, i.e. not all nucleotides that correspond with the first and second strand as defined above are base paired, and the first and second strand are often not of the same length. How to use miRNA precursor molecules as scaffolds for any selected target RNA sequence and substantially complementary first RNA sequence is described e.g. in Liu Y P Nucleic Acids Res. 2008 May; 36(9):281 1-24, which is incorporated herein by reference.

A pri-miRNA can be processed by the RNAi machinery of the cell. The pri-miRNA comprising flanking sequences at the 5′-end and the 3′-end of a pre-miRNA hairpin and/or shRNA like molecule. Such a pri-miRNA hairpin can be processed by Drosha to produce a pre-miRNA. The length of the flanking sequences can vary but may be around 80 nt in length (Zeng and Cullen, J Biol Chem. 2005 Jul. 29; 280(30):27595-603; Cullen, Mol Cell. 2004 Dec. 22; 16(6):861-5). In one embodiment, the pri-miRNA scaffold carrying the first and second RNA sequence according to the invention has a 5′-sequence flank and a 3′ sequence flank relative to the predicted pre-miRNA structure of at least 5, at least 10, at least 15, at least 20, at least 30, at least 40, or at least 50 nucleotides. Preferably, the pri-miRNA derived flanking sequences (5′ and 3′) comprised in the miRNA scaffold are derived from the same naturally occurring pri-miRNA sequence. Preferably, pre-miRNA and/or the pri-miRNA derived flanking sequences (5′ and 3′) and/or loop sequences comprised in the miRNA scaffold are derived from the same naturally occurring pri-miRNA sequence, e.g. as shown and listed in table 5 for miR451 derived scaffolds. As the (putative) guide strand RNA as comprised in the endogenous miRNA sequence can be replaced by a sequence including (or consisting of) the first RNA sequence, and the passenger strand sequence can be replaced by a sequence including (or consisting of) the second RNA sequence, it is understood that flanking sequences and/or loop sequences of the pri-miRNA or pre-miRNA sequences of the endogenous sequence may include minor sequence modifications such that the predicted structure of the scaffold miRNA sequence (e.g. M-fold predicted structure) is the same as the predicted structure of the endogenous miRNA sequence.

The first and second RNA sequence, which can form a double stranded RNA, of the invention are preferably encoded by an expression cassette. It is understood that when the double stranded RNA is to be e.g. an siRNA, consisting of two RNA strands, that there may be two expression cassettes required. One encoding an RNA strand comprising the first RNA sequence, the other cassette encoding an RNA strand comprising the second RNA strand. When the double stranded RNA is comprised in a single RNA molecule, e.g. encoding a shRNA, pre-miRNA or pri-miRNA, one expression cassette may suffice. A pol II expression cassette may comprise a promoter sequence, a sequence encoding the RNA to be expressed followed by a polyadenylation sequence. The double stranded RNA that is expressed, when comprised e.g. in a pri-miRNA scaffold, may encode for intron sequences and exon sequences and 5′-UTR's and 3′-UTRs. A pol III expression cassette in general may comprise a promoter sequence, followed by a sequence encoding the RNA (e.g. shRNA sequence, pre-miRNA, or a strand of the double stranded RNAs to be comprised in e.g. an siRNA or extended siRNA) and followed by e.g. a poly T sequence. A pol I expression cassette may comprise a pol I promoter, followed by the RNA encoding sequence and a 3′-Box. Expression cassettes for double stranded RNAs are known in the art, and any type of expression cassette can suffice, e.g. one may use a pol III promoter, a pol II promoter or a pol I promoter (i.a. ter Brake et al., Mol Ther. 2008 March; 16(3):557-64, Maczuga et al., BMC Biotechnol. 2012 Jul. 24; 12:42).

As is clear from the above, the first and second RNA sequence that are comprised in a double stranded RNA can contain additional nucleotides and/or nucleotide sequences. The double stranded RNA may be comprised in a single RNA sequence or comprised in two separate RNA strands. Whatever design is used, it is designed such that from the first and second RNA sequence an antisense RNA molecule comprising the first RNA sequence, in whole or a substantial part thereof, of the invention can be processed by the RNAi machinery such that it is incorporated in the RISC complex to have its action, i.e. to induce RNAi e.g. against the RNA target sequence comprised in an RNA encoded by a human ATXN3 gene. The sequence comprising or consisting of the first RNA sequence, in whole or a substantial part thereof, being capable of sequence specifically targeting RNA encoded by a human ATXN3 gene. Hence, as long as the double stranded RNA is capable of inducing RNAi, such a double stranded RNA is contemplated in the invention. In one embodiment, the double stranded RNA according to the invention is comprised in a pre-miRNA scaffold, a pri-miRNA scaffold, a shRNA, or an siRNA. Preferably the first and second RNA sequence as encoded by the expressed cassette are to be contained in a single transcript. It is understood that the expressed transcript in subsequent processing, i.e. cleavage, results in the single transcript being processed into multiple separate RNA molecules.

The first and second nucleotide sequences that are substantially complementary preferably do not form a double stranded RNA of 30 consecutive base pairs or longer, as these can trigger an innate immune response via the double-stranded RNA (dsRNA)-activated protein kinase pathway. Hence, the double stranded RNA has preferably less than 30 consecutive base pairs. Preferably, a pre-miRNA scaffold, a pri-miRNA scaffold, a shRNA, or an siRNA such as designed in accordance with the invention comprising the first and second RNA sequence as described herein does not comprise 30 consecutive base pairs.

The term ‘complementary’ is herein defined as nucleotides of a nucleic acid sequence that can bind to another nucleic acid sequence through hydrogen bonds, i.e. nucleotides that are capable of base pairing. Ribonucleotides, the building blocks of RNA are composed of monomers (nucleotides) containing a sugar, phosphate and a base that is either a purine (guanine, adenine) or pyrimidine (uracil, cytosine). Complementary RNA strands form double stranded RNA. A double stranded RNA may be formed from two separate complementary RNA strands or the two complementary RNA strands may be comprised in one RNA strand. In complementary RNA strands, the nucleotides cytosine and guanine (C and G) can form a base pair, guanine and uracil (G and U), and uracil and adenine (U and A) can form a base pair as well. The term substantial complementarity means that it is not required to have the first and second RNA sequence to be fully complementary, or to have the first RNA sequence and target RNA sequence or sequences of RNA encoded by a human ATXN3 gene to be fully complementary.

The substantial complementarity between the first RNA sequence and the target RNA sequence preferably consists of at most two mismatched nucleotides, more preferably having one mismatched nucleotide, most preferably having no mismatches. It is understood that one mismatched nucleotide means that over the entire length of the first RNA sequence when base paired with the target RNA sequence one nucleotide does not base pair with the target RNA sequence. Having no mismatches means that all nucleotides of the first RNA sequence base pair with the target RNA sequence, having 2 mismatches means two nucleotides of the first RNA sequence do not base pair with the target RNA sequence.

The first RNA sequence may also comprise additional nucleotides that do not have to be complementarity to the target RNA sequence and may be longer than e.g. 22 nucleotides. In such a scenario, the substantial complementarity is determined over the entire length of the target RNA sequence. In other words, when the first RNA sequence is base paired with an RNA comprising its target sequence, i.e. the target sequence that was selected and for which a first RNA sequence was selected, the substantial complementarity can be determined over the entire length of the selected target RNA sequence. As shown in the example section, a first RNA sequence was designed of 22 nucleotides to be fully complementary to a particular target RNA sequence (see table 1) and incorporated into a miRNA scaffold. Upon processing of the expressed miRNA scaffold in the cell, RNA molecules were generated by the cell comprising part or all of the first RNA sequence, some RNA molecules retained several nucleotides of the scaffold (i.e. part of the second RNA sequence). The length of such generated RNA molecules thus extending beyond the first RNA sequence length as designed. Such additional nucleotides are understood not to be taken into account when determining the substantial complementarity. Using a scaffold based on the microRNA 451a (miRbase reference number MI0001729, and as described in the examples and i.a. in WO2011133889), the substantial complementarity is to be determined over the first 22 nucleotides starting at the 5′-end which represent the first RNA sequence as so designed (see e.g. table 2). This means that the target RNA sequence may have either no, one or two mismatches over its entire length when base paired with the first RNA sequence.

As shown in the example section, double stranded RNAs designed to comprise a first nucleotide sequence length of 22 nucleotides, were tested. These first RNA sequences were designed to not have mismatches and were fully complementary with the target RNA sequence. Having a few mismatches between the first nucleotide sequence and the target RNA sequence may however be allowed according to the invention, as long as the double stranded RNA according to the invention is capable of reducing expression of transcripts comprising the target RNA sequence, such as a luciferase reporter or e.g. a transcript comprising the target RNA sequence. In this embodiment, substantial complementarity between the first RNA sequence and the target RNA sequence consists of having no, one or two mismatches over the entire length of either the first RNA sequence or the target RNA sequence encoded by an RNA of the human ATXN3 gene, whichever is the shortest.

As said, a mismatch according to the invention means that a nucleotide of the first RNA sequence does not base pair with the target RNA sequence encoded by an RNA of the human ATXN3 gene. Nucleotides that do not base pair are A and A, G and G, C and C, U and U, A and C, C and U, or A and G. A mismatch may also result from a deletion of a nucleotide, or an insertion of a nucleotide. When the mismatch is a deletion in the first RNA sequence, this means that a nucleotide of the target RNA sequence is not base paired with the first RNA sequence when compared with the entire length of the first RNA sequence. Nucleotides that can base pair are A-U, G-C and G-U. A G-U base pair is also referred to as a G-U wobble or wobble base pair. In one embodiment the number of G-U base pairs between the first RNA sequence and the target RNA sequence is 0, 1 or 2 or more. This means that when a target RNA sequence comprises a U at a position, the first RNA sequence may comprise either an A or a G at the opposite position to form a G-U or an A-U base pair. This also means that when a target RNA sequence comprises a G at a position, the first RNA sequence may comprise either a C or U at the opposite position to form a G-C or G-U base pair.

In one embodiment, there are no mismatches between the first RNA sequence and the target RNA sequence, and one or more G-U base pairs are allowed. There may be no G-U base pairs between the first RNA sequence and the target RNA sequence, or the first RNA sequence and the target RNA sequence only have base pairs that are A-U or G-C. In a preferred embodiment, there are no G-U base pairs and no mismatches between the first RNA sequence and the target RNA sequence. The first RNA sequence of the double stranded RNA according to invention preferably is fully complementary to the target RNA sequence, said complementarity consisting of G-U, G-C and A-U base pairs. The first RNA sequence of the double stranded RNA according to invention more preferably may be fully complementary to the target RNA sequence, said complementarity consisting of G-C and A-U base pairs.

In one embodiment the first RNA sequence and the target RNA sequence have at least 15, 16, 17, 18, or 19 nucleotides that base pair. Preferably the first RNA sequence and the target RNA sequence are substantially complementary, said complementarity comprising at least 19 base pairs. In another embodiment, the first RNA sequence has at least 8, 9, 10, 11, 12, 13 or 14 consecutive nucleotides that base pair with consecutive nucleotides of the target RNA sequence. In another embodiment, the first RNA sequence has at least 19 consecutive nucleotides that base pair with consecutive nucleotides of the target RNA sequence. In another embodiment the first RNA sequence comprises at least 19 consecutive nucleotides that base pair with 19 consecutive nucleotides of the target RNA sequence. In still another embodiment, the first RNA sequence has at least 17 nucleotides that base pair with the target RNA sequence and has at least 15 consecutive nucleotides that base pair with consecutive nucleotides of the target RNA sequence. The sequence length of the first nucleotide is preferably at most 21, 22, 23, 24, 25, 26, or 27 nucleotides. In another embodiment, the first RNA sequence has at least 20 consecutive nucleotides that base pair with 20 consecutive nucleotides of the target RNA sequence. In another embodiment the first RNA sequence comprises at least 21 consecutive nucleotides that base pair with 21 consecutive nucleotides of the target RNA sequence.

As said, it may be not required to have full complementarity (i.e. full base pairing (no mismatches) and no G-U base pairs) between the first RNA sequence and the target RNA sequence as such a first RNA sequence can still allow for sufficient suppression of gene expression. Also, not having full complementarity may be contemplated for example to avoid or reduce off-target RNA sequence specific gene suppression (by the RNA strand comprising the first RNA sequence and/or the RNA strand comprising the second RNA sequence) while maintaining sequence specific inhibition of transcripts comprising the target RNA sequence. However, it may be preferred to have full complementarity as it may result in more potent inhibition. Having full complementarity between the first RNA sequence and the target RNA sequence may allow for the activated RISC complex comprising said first RNA sequence (or a substantial part thereof) to cleave its target RNA sequence, whereas having mismatches may hamper cleavage and can result in mainly allowing inhibition of translation, of which the latter may result in less potent inhibition.

With regard to the second RNA sequence, this second RNA sequence is substantially complementary with the first RNA sequence. The second RNA sequence combined with the first RNA sequence forms a double stranded RNA. As said, this is to form a suitable substrate for the RNA interference machinery such that a guide sequence derived from the first RNA sequence is comprised in the RISC complex in order to e.g. sequence specifically inhibit expression of its target RNA encoded by a human ATXN3 gene. The sequence of the second RNA sequence has sequence similarity with the target RNA sequence. However, the substantial complementarity of the second RNA sequence with the first RNA sequence may be selected to have less substantial complementarity as compared with the substantial complementarity between the first RNA sequence and the target RNA sequence. Hence, the second RNA sequence may comprise 0, 1, 2, 3, 4, or more mismatches, 0, 1, 2, 3, 4 or more G-U wobble base pairs, and may comprise insertions of 0, 1, 2, 3, 4, nucleotides and/or deletions of 0, 1, 2, 3, 4, nucleotides. Preferably the first RNA sequence and the second RNA sequence are substantially complementary, said complementarity comprising 0, 1, 2, 3, or 4 G-U base pairs and/or wherein said complementarity comprises at least 17 base pairs. These mismatches, G-U wobble base pairs, insertions and deletions, are with regard to the first RNA sequence, i.e. the double stranded region that is formed between the first and second RNA sequence. As long as the first and second RNA sequence can substantially base pair and are capable of inducing sequence specific inhibition of an RNA encoded by a human ATXN3 gene, such substantial complementarity is allowed according to the invention. It is also understood that substantially complementarity between the first RNA sequence and the second RNA sequence may depend on the double stranded RNA design of choice. It may depend for example on the miRNA scaffold that is chosen for in which the double stranded RNA is to be incorporated.

As is clear from the above, the substantial complementarity between the first RNA sequence and the second RNA sequence, may comprise mismatches, deletions and/or insertions relative to a first and second RNA sequence being fully complementary (i.e. fully base paired). In one embodiment, the first and second RNA sequences have at least 11 consecutive base pairs. Hence, at least 11 consecutive nucleotides of the first RNA sequence and at least 11 consecutive nucleotides of the second RNA sequence are fully complementary. In another embodiment the first and second RNA sequence have at least 15 nucleotides that base pair. Said base pairing between at least 15 nucleotides of the first RNA sequence and at least 15 nucleotides of the second RNA sequence may consist of G-U, G-C and A-U base pairs, or may consist of G-C and A-U base pairs. In another embodiment, the first and second RNA sequence have at least 15 nucleotides that base pair and have at least 11 consecutive base pairs. In another embodiment, the first RNA sequence and the second RNA sequence are substantially complementary, wherein said complementarity comprises at least 17 base pairs. Said 17 base pairs may preferably be 17 consecutive base pairs, said base pairing consisting of G-U, G-C and A-U base pairs or consisting of G-C and A-U base pairs.

As said, the current invention provides also for an expression cassette encoding a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human ATXN3 gene. Preferably, said first RNA sequence is substantially complementary, or is complementary, to a target RNA sequence comprised in a region of the RNA encoded by a human ATXN3 gene that is 5′ of the CAG repeat region. Preferably, said target RNA sequence is present in both RNAs as expressed by both human ATXN3 alleles as present in the cell in a so called total knock down approach as opposed to a selective knockdown approach aimed at reducing only RNAs comprising CAG expansions associated with disease.

Preferably, the sequence targeted is found in the region corresponding with nucleotides 1-941 of SEQ ID NO.2. SEQ ID NO. 2 is depicted in FIG. 1. The sequence depicted in FIG. 1 represents a DNA sequence. Said DNA sequence encoding a spliced mRNA of the ATXN3 gene, a reference gene sequence for the ATXN3 gene is provided by SEQ ID NO.1 (i.e. NCBI Reference Sequence: NG_008198.2). This reference sequence, i.e. SEQ ID NO.1, comprises the exon 1-10 sequence corresponding with the exon 1-10 sequences of SEQ ID NO.2 and shown in FIG. 1, i.e. Exon 1 corresponds with nts. 5001-5093; Exon 2 corresponds with nts. 14784-14948; Exon 3 corresponds with nts. 15485-15529; Exon 4 corresponds with nts. 17791-17876; Exon 5 corresponds with nts. 18304-18370; Exon 6 corresponds with nts. 22805-22892; Exon 7 corresponds with nts. 28364-28496; Exon 8 corresponds with nts. 29156-29322; Exon 9 corresponds with nts. 30561-30657; Exon 10 corresponds with nts. 40569-40687, and also comprises the sequence of Exon 11, which corresponds with nts. 47208-53070. It is understood that wherever herein reference is made to targeting a sequence corresponding or comprised within a DNA sequence, said targeting is of the RNA that is encoded by said DNA sequence, i.e. the same sequence as listed in FIG. 1 and SEQ ID NO.2, represented by the same code but having at positions with a T a U instead.

In the sequence depicted in FIG. 1, a preferred target sequence corresponds with a target sequence comprised in one of exons 5, 6, 7, 8, and 9. More preferred the target sequence corresponds with a target sequence comprised in one of exons 6, 7, 8, and 9, or even more preferred in one of exons 7, 8 and 9. The sequences of exons 5, 6, 7, 6, and 9, corresponding respectively with nucleotides 390-456, 457-544, 545-677, 678-844, and 845-941 of SEQ ID NO.2. It is understood that the ATXN3 transcripts have different exon compositions, due to alternative splicing and thus not all transcripts have the same exon composition, i.e. one or more exons as depicted in FIG. 1 may be missing and/or alternative splice sites may be used. However, the sequences corresponding with exons 5, 6, 7, 8 and 9, as depicted in FIG. 1 and corresponding with nucleotides 390-941 of SEQ ID NO.2 are comprised in most ATXN3 transcripts.

As ATXN3 transcript variants may have slightly different exon compositions, targeting variant transcript sequences is also encompassed by the present invention, as long as the target sequence is comprised in the 550 nucleotides found directly 3′ from the CAG repeat of spliced ATXN3 transcripts, such a target sequence may be contemplated in accordance with the invention. As ATXN3 transcript variants may have slightly different exon compositions, targeting variant transcript sequences is also encompassed by the present invention, as long as the target sequence is comprised in one or two of the sequences of exons 5, 6, 7, 6, and 9, corresponding respectively with nucleotides 390-456, 457-544, 545-677, 678-844, and 845-941 of SEQ ID NO.2, such a target sequence may be contemplated in accordance with the invention. It is understood that when two of the exon sequences are targeted, this may encompass a target sequence that is at the splice junction (the site where to exons are joined). This is because, as shown in the examples, in the regions 5′ from the CAG repeat highly efficacious target sequences for reducing ATXN3 gene expression are to be found. Said first and second RNA sequences in accordance with the invention, when expressed in a cell can reduce expression of RNA encoded by a human ATXN3 gene both in the cell nucleus as in the cytoplasm. Target RNA sequences may be selected to be comprised in spliced and unspliced RNAs as expressed from the human ATXN3 gene. Hence, preferably, ATXN3 transcripts are targeted by selecting a target sequence comprised in the sequence ranging from the sequence corresponding with 390-456 of SEQ ID NO.2 (exon 5 as depicted in FIG. 1) to the sequence corresponding with 845-941 of SEQ ID NO.2 (exon 9 as depicted in FIG. 1), as encoded by SEQ ID NO.1 or as encoded by SEQ ID NO.2. It is understood that in this range the exon 5 and exon 9 sequence are included. ATXN3 transcripts may further be targeted by selecting a target sequence comprised in the sequence ranging from the sequence corresponding with 457-544 of SEQ ID NO.2 (exon 6 as depicted in FIG. 1) to the sequence corresponding with 845-941 of SEQ ID NO.2 (exon 9 as depicted in FIG. 1), as encoded by SEQ ID NO.1 or as encoded by SEQ ID NO.2. It is understood that in this range the exon 6 and exon 9 sequence are included.

Some target RNA sequences may only target spliced RNAs because the target sequence is comprised in adjacent exons, such as e.g. SEQ ID NO. 10 and SEQ ID NO. 11. Hence, target RNA sequences may be selected to target a sequence corresponding with nucleotides 828-862 of SEQ ID NO. 2 corresponding with the splice junction of exon 8-exon 9, or with nucleotides 439-473 of SEQ ID NO. 2 corresponding with the splice junction of exon 5-exon 6. Preferably a sequence is targeted comprised in a sequence corresponding with exons 5, 6, 8 and 9 as depicted in FIG. 1. Such a sequence may comprise a splice junction between exons 5 and 6, and a splice junction between exons 8 and 9. More preferably, a target RNA sequence is comprised in a sequence of exon 9 as depicted in FIG. 1. Most preferably, a target RNA sequence is comprised in a splice junction between exons 8 and 9 as depicted in FIG. 1.

Accordingly, target RNA sequences that may be suitable are listed in table 1 below. Hence, in one embodiment, an expression cassette is provided encoding a first RNA sequence and a second RNA sequence wherein the first and second RNA sequence are substantially complementary, wherein the first RNA sequence has a sequence length of at least 19 nucleotides and is substantially complementary to a target RNA sequence selected from the group listed in table 1 comprised in an RNA encoded by a human ATXN3 gene.

Selected target RNA sequences are preferably as listed in table 1 below.

TABLE 1
Selected Target nucleotide sequences.

| SEQ ID | TARGET RNA SEQUENCE | |
|---|---|---|
| NO. | (5′-NNNN-3′) | length |
| 3 | GCCGUUGGCUCCAGACAAAUAA | 22 |
| 4 | AAUAAACAUGGAGUCCAUCUUC | 22 |
| 5 | UACAGCAGCCUUCUGGAAAUAU | 22 |
| 6 | CUUCUGGAAAUAUGGAUGACAG | 22 |
| 7 | AAGUUUGGGGUUUAGAACUAAU | 22 |
| 8 | AACUAAUCCUGUUCAACAGUCC | 22 |
| 9 | AACACUGGUUUACAGUUAGAAA | 22 |
| 10 | AAUUAGGAAAACAGUGGUUUAA | 22 |
| 11 | AAGUAUGCAAGGUAGUUCCAGA | 22 |
| 12 | UACUUCAGAAGAGCUUCGGAAG | 22 |
| 13 | GAGACGAGAAGCCUACUUUGAA | 22 |

For SEQ ID NOs. 3-13, start position to endposition, and target
exon (SEQ ID NO. N: x-y, exon z) in ATXN3 NCBI Reference Sequence: NM_004993.5 (SEQ ID NO. 2), are:
SEQ ID NO 3: 46-67, exon 1; SEQ ID NO 4: 63-84, exon 1; SEQ ID NO 5: 254-275, exon 2-3; SEQ ID NO 6:
263-284, exon 3; SEQ ID NO 7: 323-244, exon 4; SEQ ID NO 8: 338-359, exon 4; SEQ ID NO 9: 422-443.
exon 5; SEQ ID NO 10: 443-464, exon 5-6; SEQ ID NO 11: 834-855, exon 8-9; SEQ ID NO 12: 897-918,
exon 9; SEQ ID NO 13: 918-939, exon 9).

From these target RNA sequences it was surprisingly found that highly advantageous suitable first and second RNA sequences could be made in accordance with the invention to provide for an expression cassette encoding said first RNA sequence and said second RNA sequence, wherein the first and second RNA sequence are substantially complementary, wherein the first RNA sequence has a sequence length of at least 19 nucleotides and is substantially complementary to one of said target RNA sequences to highly efficiently induce RNAi to reduce ATXN3 gene expression.

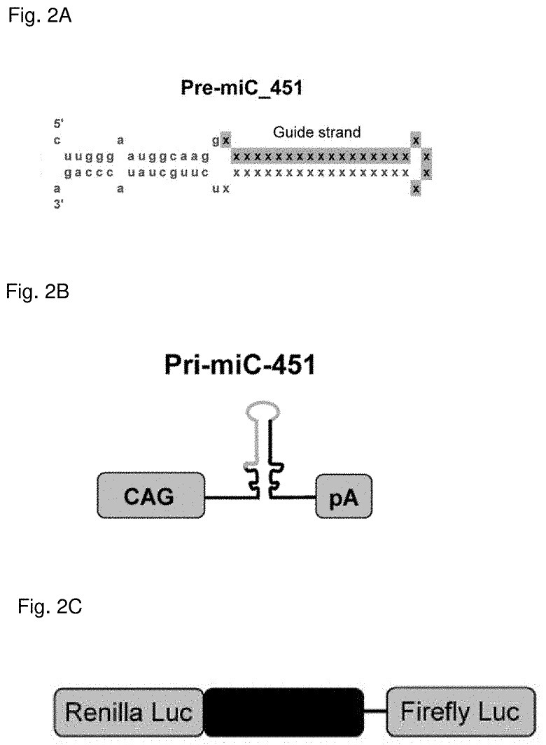

As shown in the examples, the first and second RNA sequence of the invention, may be preferably incorporated in a pre-miRNA or a pri-miRNA scaffold derived from microRNA 451a. The terms ‘microRNA451a’, ‘miR451’, ‘451 scaffold’ or simply ‘451’ are used interchangeably throughout this specification. A pri-miRNA scaffold for miR451 is depicted in FIG. 2a. This scaffold allows to induce RNA interference resulting in only guide strand induced RNA interference. The pri-miR451 scaffold does not result in a passenger strand because the processing is different from the canonical miRNA processing pathway (Cheloufi et al., 2010 Jun. 3; 465(7298):584-9 and Yang et al., Proc Natl Acad Sci USA. 2010 Aug. 24; 107(34):15163-8). Hence, this scaffold represents an excellent candidate to develop a gene therapy product as unwanted potential off-targeting by passenger strands can be largely, if not completely, avoided. As an alternative to the miR451 scaffold, similar Dicer independent structures may be employed such as described herein and i.a. in Herrera-Carrillo and Berkhout, NAR, 2017, Vol. 45 No. 18 10369-79, which is incorporated herein by reference. As a passenger strand may result in off-targeting e.g. targeting transcripts other than ATXN3 RNA, using such a scaffold may allow one to avoid such unwanted targeting. Hence, it is preferred that, whichever scaffold is selected, a scaffold is selected that produces less than 5% passenger strands, more preferably less than 4%, most preferably less than 3% passenger strands. The percentage passenger strands being calculated by determining the total quantity of strands produced from an RNA scaffold comprising a sequence of at least 16 nucleotides derived from the second RNA sequence and dividing it by the total quantity of strands produced from said RNA scaffold comprising a sequence of at least 16 nucleotides derived from the second RNA sequence and the first RNA sequence as produced in human neurons e.g. as described in the example section.

As shown in the examples, a first RNA sequence of 22 nucleotides (e.g. for a miR451) in length may be selected and incorporated in a miRNA scaffold. Such a miRNA scaffold sequence is subsequently processed by the RNAi machinery as present in the cell. When reference is made to miRNA scaffold it is understood to comprise pri-miRNA structures or pre-miRNA structures. As shown in the examples, such miRNA scaffolds, when processed in a neuronal cell, result in guide sequences comprising the first RNA sequence, or a substantial part thereof, in the range 21-30 nucleotides in length for the 451 scaffold. Such guide strands being capable of reducing the human ATXN3 gene expression by targeting the selected target sequences. As is clear from the above, and as shown in the examples, the first RNA sequence as it is encoded by the expression cassette of the invention, is comprised in part or in whole, in a guide strand when it has been processed by the RNAi machinery of the cell. Hence, the guide strand that is to be generated from the RNA encoded by the expression cassette, comprising the first RNA sequence and the second RNA sequence is to comprise at least 18 nucleotides of the first RNA sequence. Preferably, such a guide strand comprises at least 19 nucleotides, 20 nucleotides, 21 nucleotides, or at least 22 nucleotides. A guide strand can comprise the first RNA sequence also as a whole. In selecting a miRNA scaffold e.g. from miRNA scaffolds as found in nature such as in humans, the first RNA sequence can be selected such that it is to replace the original guide strand. As shown in the example section, this does not necessarily mean that a guide strand produced from such an artificial scaffold are identical in length to the first RNA sequence selected, nor may it necessarily be so that the first RNA sequence is in its entirety to be found in the guide strand that is produced.

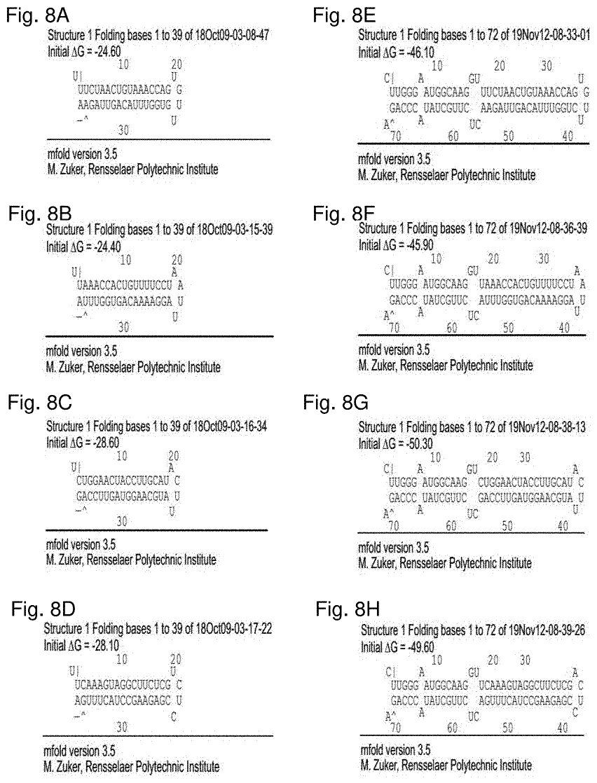

A miRNA 451 scaffold, as shown in the examples, and as shown in FIG. 2a and FIG. 8 preferably comprises from 5′ to 3′, firstly 5′-CUUGGGAAUGGCAAGG-3′ (SEQ ID NO.50), followed by a sequence of 22 nucleotides, comprising or consisting of the first RNA sequence, followed by a sequence of 17 nucleotides, which can be regarded to be the second RNA sequence, which is complementary over its entire length with nucleotides 2-18 of said sequence of 22 nucleotides, subsequently followed by sequence 5′-CUCUUGCUAUACCCAGA-3′ (SEQ ID NO.51). Preferably the first 5′-C nucleotide of the latter sequence is not to base pair with the first nucleotide of the first RNA sequence. Such a scaffold may comprise further flanking sequences as found in the original pri-miR451 scaffold. Alternatively, the flanking sequences, 5′-CUUGGGAAUGGCAAGG′-3′ (SEQ ID NO.50) and 5′-CUCUUGCUAUACCCAGA-3′ (SEQ ID NO.51) may be replaced by flanking sequences of other pri-mRNA structures. It is understood that, as the miR451 scaffold can provide for guide strands only due to the length of the stem sequence, it is preferred that alternative flanking sequences do not extend the stem length of 17 consecutive base pairs. As is clear from the above, the sequence of the scaffold may differ not only with regard to the (putative) guide strand sequence, and sequence complementary thereto, as present in the wild-type scaffold (FIG. 2a), but may also comprise additional mutations in the 5′ sequence, loop sequence and 3′ sequence as well, as additional mutations may be required to provide for an RNA structure that is predicted to mimic the secondary structure of the wild-type scaffold and/or does not have a stem extending beyond 17 consecutive base pairs. Such a scaffold may be comprised in a larger RNA transcript, e.g. a pol II expressed transcript, comprising e.g. a 5′ UTR and a 3′UTR and a poly A. Flanking structures may also be absent. An expression cassette in accordance with the invention thus expressing a shRNA-like structure having a sequence of 22 nucleotides, comprising or consisting of the first RNA sequence, followed by a sequence of 17 nucleotides, which can be regarded to be the second RNA sequence, which is complementary over its entire length with nucleotides 2-18 of said sequence of 22 nucleotides, and further comprising 1 or more additional nucleotides which is predicted not to form a base pair with the first RNA sequence. The latter shRNA-like structure derived from the miR451 scaffold can be referred to as a pre-miRNA scaffold from miR451.

In another embodiment, an expression cassette according to the invention is provided, wherein said first RNA sequence is substantially complementary to a target RNA sequence selected from the group consisting of SEQ ID NO. 9, 10, 11 or SEQ ID NO. 13. These particular target RNA sequences were found to provide for most potent inhibition of reporters and/or ATXN3 expression in human cells, such as neurons, as shown in the example section. Preferably said first RNA sequence has a length of 19, 20, 21, or 22 nucleotides. More preferably said first RNA sequence is fully complementary over its entire length with said first RNA target sequence. Most preferably said first RNA sequence has a length of 19, 20, 21, or 22 nucleotides, wherein said first RNA sequence is fully complementary over its entire length with said first RNA target sequence. Preferably, said first RNA sequence is selected from the group consisting of SEQ ID NO. 14, 15, 16 and 17.

TABLE 2
First RNA sequences

| SEQ ID | FIRST RNA SEQUENCE | |
|---|---|---|
| NO. | (5′-NNNN-3′) | length |
| 14 | UUUCUAACUGUAAACCAGUGUU | 22 |
| 15 | UUAAACCACUGUUUUCCUAAUU | 22 |
| 16 | UCUGGAACUACCUUGCAUACUU | 22 |
| 17 | UUCAAAGUAGGCUUCUCGUCUC | 22 |

Such a first RNA sequence is to be combined with a second RNA sequence. As described herein, the skilled person is well capable of designing and selecting a suitable second RNA sequence in order to provide for a first and second RNA sequence that can induce RNA interference when expressed in a cell. Suitable second RNA sequences that can be contemplated are listed below in table 3.

TABLE 3
Second RNA sequences.

| SEQ | | |
|---|---|---|
| ID | SECOND RNA SEQUENCE | |
| NO. | (5′-NNNN-3′) | length |
| 18 | CUGGUUUACAGUUAGAA | 17 |
| 19 | AGGAAAACAGUGGUUUA | 17 |
| 20 | AUGCAAGGUAGUUCCAG | 17 |
| 21 | CGAGAAGCCUACUUUGA | 17 |

Said first RNA sequence is preferably comprised in a miRNA scaffold, more preferably a miR451 scaffold, such as shown in the examples. A suitable scaffold comprising a first and second RNA sequence in accordance with the invention can be a sequence such as listed below in tables 4 and 5. The sequences as listed in table 4 may comprise further sequences and may be comprised in a pri-miRNA scaffold such as listed in table 5.

TABLE 4
pre-miRNA sequences.

| SEQ | | |
|---|---|---|
| ID | first RNA sequence - second RNA sequence | |
| NO. | [5′-NNNN-3′] | length |
| 22 | UUUCUAACUGUAAACCAGUGUUCUGGUUUACAGUUAGAA | 39 |
| 23 | UUAAACCACUGUUUUCCUAAUUAGGAAAACAGUGGUUUA | 39 |
| 24 | UCUGGAACUACCUUGCAUACUUAUGCAAGGUAGUUCCAG | 39 |
| 25 | UUCAAAGUAGGCUUCUCGUCUCCGAGAAGCCUACUUUGA | 39 |

TABLE 5
pri-miRNA sequences.

| SEQ | | |
|---|---|---|
| ID | flank - first RNA sequence - second RNA | |
| NO. | sequence - flank [5′-NNNN-3′] | length |
| 26 | CUUGGGAAUG GCAAGGUUUC UAACUGUAAA CCAGUGUUCU | 72 |
| | GGUUUACAGU UAGAACUCUU GCUAUACCCA GA | |
| 27 | CUUGGGAAUG GCAAGGUUAA ACCACUGUUU UCCUAAUUAG | 72 |
| | GAAAACAGUG GUUUACUCUU GCUAUACCCA GA | |
| 28 | CUUGGGAAUG GCAAGGUCUG GAACUACCUU GCAUACUUAU | 72 |
| | GCAAGGUAGU UCCAGCUCUU GCUAUACCCA GA | |
| 29 | CUUGGGAAUG GCAAGGUUCA AAGUAGGCUU CUCGUCUCCG | 72 |
| | AGAAGCCUAC UUUGACUCUU GCUAUACCCA GA | |

Such first RNA sequences as described above, can be comprised in expression cassettes. Such first RNA sequences can be comprised in RNA structures that are encoded by expression cassettes. Such first and second RNA sequences as described above can be comprised in expression cassettes. Such first and second RNA sequences can be comprised in RNA structures that are encoded by expression cassettes.

Accordingly, targeting target RNA sequences, which are preferably in the region 5′ from the CAG region, and which are preferably target RNA sequences such as listed in table 1, more preferably a target RNA sequence selected from SEQ ID NO. 9, 10, 11 and SEQ ID NO. 13, utilizing first and second RNA sequences as described above was found to be in particular useful for reducing expression of RNA transcripts encoded by the human ATXN3 gene.

As described above, and as shown in the examples, these target sequences were found to be in particular suitable for reducing ATXN3 gene expression via an RNAi approach that utilizes an expression cassette encoding a first RNA sequence and a second RNA sequence wherein the first and second RNA sequence are substantially complementary, wherein the first RNA sequence has a sequence length of at least 19 nucleotides and is substantially complementarity to a target RNA sequence comprised in an RNA encoded by a human ATXN3 gene.

Moreover, and in further embodiments, one or more expression cassettes are provided for combined targeting of target RNA sequences. Hence, combined targeting of RNA target sequences comprised in human ATXN3 gene transcripts is contemplated in the invention. Such combined targeting is to reduce expression of human ATXN3 gene transcripts and/orataxin-3 protein, including transcripts and proteins containing CAG expansions, even further as compared to a single targeting of target RNA sequence. Combined targeting of RNA target sequences can be obtained by providing e.g. two separate expression cassettes. Alternatively, and preferably, one expression cassette is provided that is to encode for each target a first RNA sequence combined with a second RNA sequence, such an expression cassette thus expressing a single RNA transcript comprising at least two separate first RNA sequences that can be processed by the cell to provide for two separate guide sequences, each separate guide sequence targeting one of the at least two targets, i.e. a first target RNA sequence and a second target RNA sequence. Hence, in one embodiment, one or more expression cassettes are provided for combined targeting of SEQ ID NO. 9, and 10; SEQ ID NO. 9 and 11; SEQ ID NO. 9 and 13; SEQ ID NO. 10 and 11; SEQ ID NO. 10 and 13; SEQ ID NO. 11 and 13. In another embodiment, one or more expression cassettes are provided for combined targeting of SEQ ID NO. ID NO. 9, 10 and 11; SEQ ID NO. 9, 10 and 13; SEQ ID NO. 9, 11 and 13; SEQ ID NO. 10, 11 and 13. In another embodiment, one or more expression cassettes are provided for combined targeting of SEQ ID NO.9, 10, 11 and 13. Since it is anticipated that combined targeting of RNA target sequences comprised in human ATXN3 gene transcripts may reduce expression of human ATXN3 gene transcripts and/or ataxin-3 protein, including transcripts and proteins containing CAG expansions, even further as compared to a single targeting of target RNA sequence, such combined targeting may thus significantly benefit affected human patients by slowing down or complete halting of further neuropathologies.

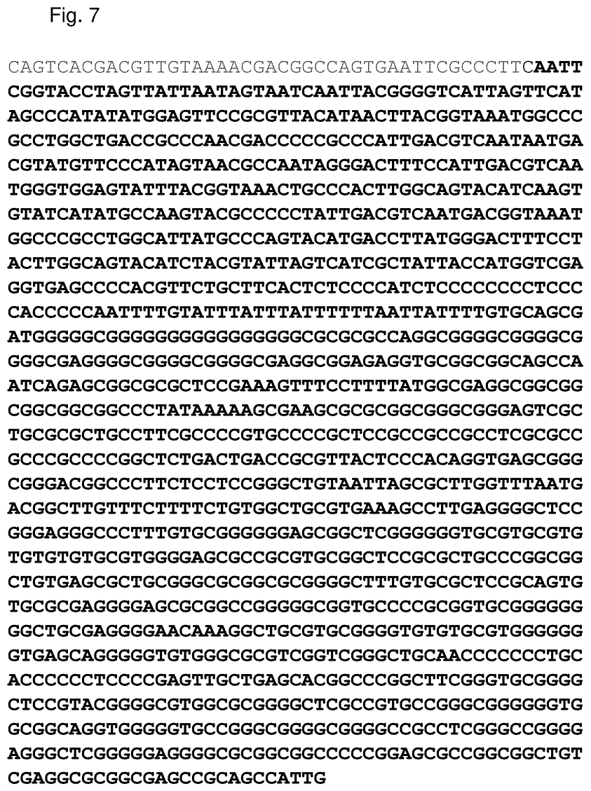

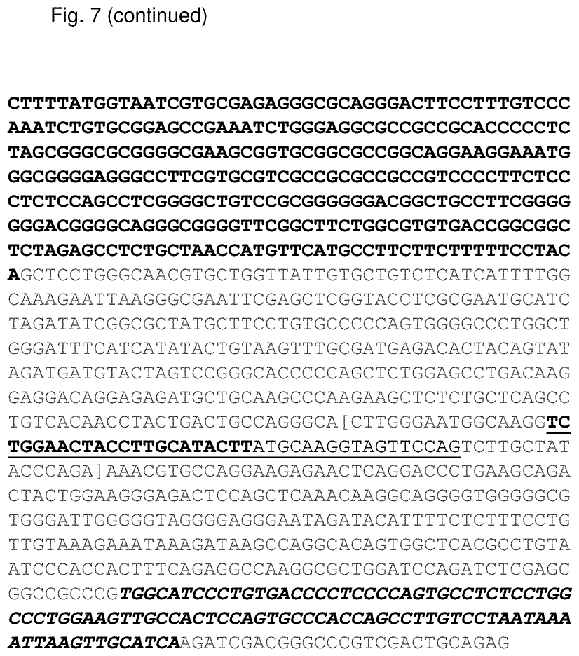

Preferably a pol II promoter is used, such as a CAG promoter (i.a. Miyazaki et al. Gene. 79 (2): 269-77; Niwa, Gene. 108 (2): 193-9) and as depicted e.g. in FIG. 2b and FIG. 7, a PGK promoter, or a CMV promoter (Such as depicted e.g. in FIG. 2 of WO2016102664, which is herein incorporated by reference). As neurons are affected in the disease, it may in particularly be useful to use a neurospecific or pan-neuronal and astrocyte-specific promoter. Examples of suitable neurospecific promoters are Neuron-Specific Enolase (NSE), human synapsin 1, caMK kinase and tubuline (Hioki et al. Gene Ther. 2007 June; 14(11):872-82). Other suitable promoters that can be contemplated are inducible or repressible promoters, i.e. a promoter that initiates transcription only when the host cell is exposed to some particular stimuli or a particular stimulus or vice versa.

Said expression cassettes according to the invention can be transferred to a cell, using e.g. transfection methods. Any suitable means may suffice to transfer an expression cassette according to the invention. Preferably, gene therapy vectors are used that stably transfer the expression cassette to the cells such that stable expression of the double stranded RNAs that induce sequence specific inhibition of the a human ATXN3 gene as described above can be achieved. Suitable vectors may be lentiviral vectors, retrotransposon based vector systems, or AAV vectors. It is understood that as e.g. lentiviral vectors carry an RNA genome, the RNA genome will encode for said expression cassette such that after transduction of a cell, said DNA sequence and said expression cassette is formed. Preferably a viral vector is used such as AAV. A preferred AAV vector that may be used is an AAV vector of serotype 5. AAV of serotype 5 (also referred to as AAV5) may be particularly useful for transducing human neurons and human astrocytes such as shown in the examples. Thus, AAV5 can efficiently transduce different human cell types of the CNS including (human induced pluripotent stem cell-derived) frontal brain-like neurons, dopaminergic neurons, motor neurons and astrocytes and AAV5 is therefore a suitable vector candidate to deliver therapeutic genes to the CNS to treat neurogenerative diseases, including SCA3. Particularly, AAV5 can be used to target human ATXN3 as described herein. The production of AAV vectors comprising any expression cassette of interest is well described e.g. in; WO2007/046703, WO2007/148971, WO2009/014445, WO2009/104964, WO2011/122950, WO2013/0361 18, which are incorporated herein in its entirety.

AAV sequences that may be used in the present invention for the production of AAV vectors, e.g. produced in insect or mammalian cell lines, can be derived from the genome of any AAV serotype. Generally, the AAV serotypes have genomic sequences of significant homology at the amino acid and the nucleic acid levels, provide an identical set of genetic functions, produce virions which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (1997, J. Vir. 71: 6823-33); Srivastava et al. (1983, J. Vir. 45:555-64); Chlorini et al. (1999, J. Vir. 73:1309-1319); Rutledge et al. (1998, J. Vir. 72:309-319); and Wu et al. (2000, J. Vir. 74: 8635-47). AAV serotypes 1, 2, 3, 4 and 5 are preferred source of AAV nucleotide sequences for use in the context of the present invention. Preferably the AAV ITR sequences for use in the context of the present invention are derived from AAV1, AAV2, and/or AAV5. Likewise, the Rep52, Rep40, Rep78 and/or Rep68 coding sequences are preferably derived from AAV1, AAV2 and AAV5. The sequences coding for the VP1, VP2, and VP3 capsid proteins for use in the context of the present invention may however be taken from any of the known 42 serotypes, more preferably from AAV1, AAV2, AAV3, AAV4, AAV5, AAV6, AAV7, AAV8 or AAV9 or newly developed AAV-like particles obtained by e.g. capsid shuffling techniques and AAV capsid libraries. AAV capsids may consist of VP1, VP2 and VP3, but may also consist of VP1 and VP3.

In another embodiment, a host cell is provided comprising the DNA sequence or expression cassette according to the invention. For example, said expression cassette or DNA sequence may be comprised in a plasmid contained in bacteria. Said expression cassette or DNA sequence may also be comprised in a production cell that produces e.g. a viral vector. Said expression cassette may also be provided in a baculovirus vector.

As shown in the example section, and as explained above, the double stranded RNA according to the invention, the DNA sequence according to invention, the expression cassette according to the invention and the gene therapy vector according to the invention are for use as a medicament, in particular for use as medicament in the treatment of SCA3. Thus, the double stranded RNA according to the invention, the DNA sequence according to invention, the expression cassette according to the invention and the gene therapy vector according to the invention are for use in a medical treatment, in particular for use in the treatment of SCA3. More particularly, use of the double stranded RNA according to the invention, the DNA sequence according to invention, the expression cassette according to the invention and the gene therapy vector according to the invention in the treatment of SCA3 is anticipated to slow down or halt neuropathologies.

In one embodiment, said use in a medical treatment comprises a reduction (also referred to as lowering) of ATXN3 mRNA expression of at least 50%, more preferably, more preferably at least 60%, more preferably of at least 65%. It is understood that a reduction of ATXN3 mRNA expression of 60% represents an ATXN3 mRNA expression which is 40% of normal ATXN3 mRNA expression. Normal ATXN3 mRNA expression representing ATXN3 mRNA expression in a cell without expressing a first and second RNA in accordance with the invention. In a further embodiment, said use of a gene therapy vector (or expression cassette) in accordance with the invention comprises a reduction of ATXN3 mRNA expression of at least 50%, more preferably, more preferably at least 60%, more preferably of at least 65%, wherein said reduction is determined in human iPSC neurons. In a further embodiment, the reduction of ATXN3 mRNA expression in human iPSC neurons is determined such as described in the examples. In another embodiment, the reduction of ATXN3 mRNA is determined in 293 T cells such as described in the examples, and preferably a reduction of ATXN3 mRNA is obtained in 293T cells of about 75% or more at the highest dose. In still another embodiment, said reduction of ATXN3 mRNA expression is as determined in vivo such as in the F512 SCA3 knock-in mouse model as shown in the examples. Said reduction of ATXN3 mRNA expression preferably comprises a reduction of ATXN3 mRNA expression e.g. as determined using e.g. RT-qPCR or the like. Said reduction of ATXN3 mRNA expression is preferably in the brain stem and/or cerebellum.

In another embodiment, as shown in the example section, said use in a medical treatment comprises a reduction (also referred to as lowering) of ATXN3 protein expression of at least 50%, more preferably at least 60%, more preferably of at least 65%. It is understood that a reduction of ATXN3 protein expression of 60% represents an ATXN3 protein expression which is 40% of normal ATXN3 protein expression. Said reduction may provide for a reduction of ATXN3 protein aggregates, which may be a reduction in soluble and insoluble aggregates. Said reduction may provide for a reduction in ataxin-3 nuclear inclusions. Normal ATXN3 protein expression representing ATXN3 protein expression in a cell without expressing a first and second RNA in accordance with the invention. In another embodiment, said reduction of ATXN3 protein is as determined in 293 T cells such as described in the examples, which is a reduction of about 75%. In another embodiment said reduction of ATXN3 protein expression is as determined in the F512 SCA3 knock-in mouse model as shown in the examples. Said reduction of ATXN3 protein expression preferably comprising a reduction of ATXN3 protein expression as determined using Time-resolved fluorescence energy transfer (TR-FRET) immunoassay (Nguyen et al., PLOS ONE, April 2013, Vol. 8 Issue 4 e62043). Said reduction of ATXN-3, reduction of ATXN-3 aggregates and/or nuclear inclusions may also be a reduction as observed in a mouse model comprising injecting a mixture of lentiviral vectors (encoding mutant ataxin-3 (atx3-72Q)) and AAV5-miATXN3, such as described in the example section. Said reduction of ATXN3 protein expression is preferably in the brain stem and/or cerebellum.

As said, it is understood that the first RNA sequence in accordance with the invention is to be comprised, in whole or a substantial part thereof, in a guide strand when expressed in and subsequently processed by a cell. In another embodiment, in accordance with the invention, said first RNA sequence and said second RNA sequence, when expressed in a cell, are processed by the cell to produce a guide sequence comprising the first RNA sequence, wherein said guide sequences comprise at most 15% of the total miRNA counts as produced by the cell. More preferably, said guide sequences comprise at most 10%, more preferably at most 8%, most preferably at most 6% of the total miRNA counts as produced by the cell. Said guide sequences representing the sequences produced by the cell comprising, in whole or a substantial part thereof, the first RNA sequences as assessed e.g. by sequence identity with determined sequences with the first RNA sequence. The total miRNA count referring to the number of sequences representing the endogenous miRNA sequences combined with the number of sequences comprising the first RNA sequences. Examples of sequences as determined by high throughput sequencing representing guide sequences comprising the first RNA sequence, in whole or a substantial part thereof, are shown in the tables below. Said percentage of total miRNA of first RNA sequence derived guide sequences is preferably determined in iPSC cells. In another embodiment, said percentage of total miRNA of first RNA sequence derived guide sequences is determined in iPSC cells as shown in the examples. Delivery to the CNS may comprise intraparenchymal injections (Samaranch et al., Gene Ther. 2017 April; 24(4):253-261). Said intraparenchymal delivery may also comprise intrastriatal or intrathalamic injections, or intracerebellar injections including injections into the deep cerebellar nuclei for example. Said CNS delivery may also comprise delivery to the cerebrospinal fluid (CSF) upon which affected CNS regions may be effectively transduced as the vector can reach affected areas in the disease, such as the cerebellum and/or the brain stem, via diffusion of the cerebrospinal fluid into these areas.

Such delivery methods representing an efficient way to deliver the gene therapy vector to the CNS, including affected brain stem and/or cerebellum to target affected neurons. Such injections are preferably carried out through MRI-guided injections. Said methods of treatments are in particular useful for human subjects having SCA3.

Delivery to the CNS may comprise intra-CSF administration. Intra-CSF delivery methods representing an efficient way to deliver the gene therapy vector to the CNS, including affected brain stem and/or cerebellum to target affected neurons. CNS delivery in further embodiments may also comprise intrathecal injections (e.g. WO2015060722; Bailey et al., Mol Ther Methods Clin Dev. 2018 Feb. 15; 9:160-171;), intra cisterna magna injections and/or subpial injections (Miyanohara et al., Mol Ther Methods Clin Dev. 2016 Jul. 13; 3:16046.) of the vector. CNS delivery may also comprise intracerebroventricular (ICV) or intrastriatal injections. Preferably, the delivery does not comprise intraparenchymal injections, as such delivery routes may have a risk of inducing injury. CNS delivery may also comprise a combination of two or more of any of the above listed CNS delivery methods. For example, intrathecal or subpial injection may be combined with intracerebroventricular and/or intra cisterna magna injections. Intrathecal or subpial injection may also be combined with intraparenchymal injections. Said combination of methods can be simultaneous, i.e. at the same time, or sequential, i.e. within a certain time interval. Said methods of treatments are in particular useful for human subjects having SCA3. As the brain stem has a highly complex structure, it is also contemplated to deliver the gene therapy vector in close (physical) proximity to this brain area such that the gene therapy vector can reach this area without requiring to inject directly into this area with which high risks may be associated.

It is understood that the treatment of SCA3 involves human subjects having SCA3 including human subjects having a genetic predisposition of developing SCA3 that do not yet show signs of the disease. Hence, the treatment of human subjects with SCA3 includes the treatment of any human subject carrying an ATXN3 gene with a CAG expansion associated with SCA3. It is anticipated that said treatment involves the slowing down and/or halting of neuropathology associated with RNAs containing expanded CAG repeats and/or of ataxin-3 protein containing expanded polyQ translated therefrom. In one embodiment, the said treatment results in a reduction size of brain lesions associated with SCA3 mouse models. In another embodiment, said treatment results in a reduction of ATXN-3 protein aggregates, associated with SCA3. Patients may thus benefit from treatment with the gene therapy vectors and/or expression cassettes according to the present invention and may show amelioration of motor impairment and prolonged survival.

### Examples

Design of miRNAs Targeting 5′ Region of ATXN3

We selected target sites for a total silencing approach (see FIG. 1). Selected target sequences are listed in table 1 above. First RNA sequences that were used to replace the endogenous guide strand sequence in the miRNA scaffolds were fully complementary to the target sequences of table 1, having standard Watson-Crick base pairing (G-C and A-U). Sequences were incorporated into human pri-miRNA miR-451 scaffold sequences. 200 nt 5′ and 3′ flanking regions were included and the mfold program (unafold.rna.albany.edu/?q=mfold) was used with standard settings to determine whether the candidates are folded into the secondary structures as depicted in FIG. 8. If not folded into the predicted secondary structure, the sequence was adapted, which did not involve adapting the first RNA sequences, such that the correct structure was folded by the program. Complete scaffold encoding DNA sequences were subsequently ordered from GeneArt gene synthesis (Invitrogen) and were subsequently cloned into an expression vector containing the CMV immediate-early enhancer fused to chicken β-actin (CAG) promoter (Inovio, Plymouth Meeting, Pa.), an example of which is depicted in FIG. 7.

In Vitro Testing of miR451 Scaffold Constructs on Reporter Systems

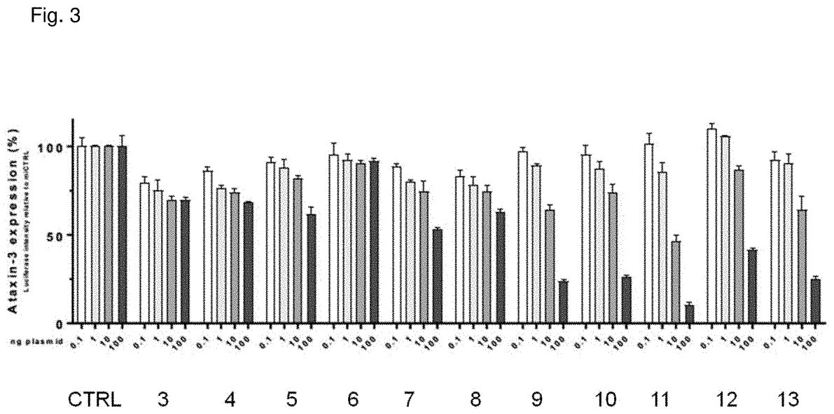

To test the efficacy of the miATXN3 candidates, we designed Luc reporters bearing complementary ATXN3 target regions fused to the Renilla luciferase (RL) gene (FIG. 2c). Target sequences were synthesized (GeneArt) and cloned in the 3′UTR of the Renilla luciferase (RL) gene of the psiCHECK-2 vector (Promega, Madison, Wis.). The firefly luciferase (FL) gene was also expressed in this vector and served as internal control. Co-transfections of reporters and constructs, with increasing amounts of 0.1, 1, 10 and 100 ng, were carried out in 293T cells using Lipofectamine using standard culture and transfection conditions and in accordance with manufacturer's instructions. 48 hours post-transfection, cells were lysed in passive lysis buffer (Promega) at room temperature, and FL and RL activities were measured in lysate with the Dual-Luciferase Reporter Assay System (Promega). Relative luciferase activity was calculated as the ratio between RL and FL activities. The results (FIG. 3) indicate that the 5′ region, in particular from, and including, exon 5 to, and not including exon 10, is a good region for highly efficient targeting of ATXN3 gene expression, as the most efficient knock-down was obtained in this region (target sequences SEQ ID NOs. 9-13).

### In Vitro Testing—Knockdown of Endogenous Ataxin-3 Protein

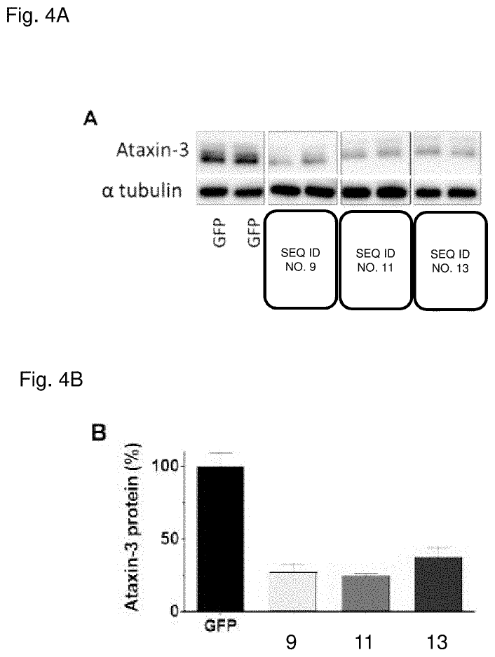

The ability to silence endogenously expressed ATXN3 mRNA and ataxin-3 protein was tested in HEK293T cells. miATXN3 candidates, targeting SEQ ID NO. 9, 11 and 13 were transfected, with a GFP expression cassette as control. Protein was isolated three days post transfection. Subsequently, western blots were carried out. Blotted proteins were stained for ataxin-3 and α-tubulin was used as loading control (FIG. 4a). Ataxin-3 protein levels were measured relative to green fluorescent protein (GFP) control, which was set at 100%. A one-way ANOVA showed a significant difference between the expression of GFP transfected cells and the expression of candidates P<0.0001, with a reduction up to 75% (FIG. 4b). Two bands were visible for ataxin-3, and both bands were reduced, indicating that alleles of different lengths were targeted.

Dose Dependent ATXN3 Lowering in Neuronal Cultures Transduced with ATXN3 miRNA

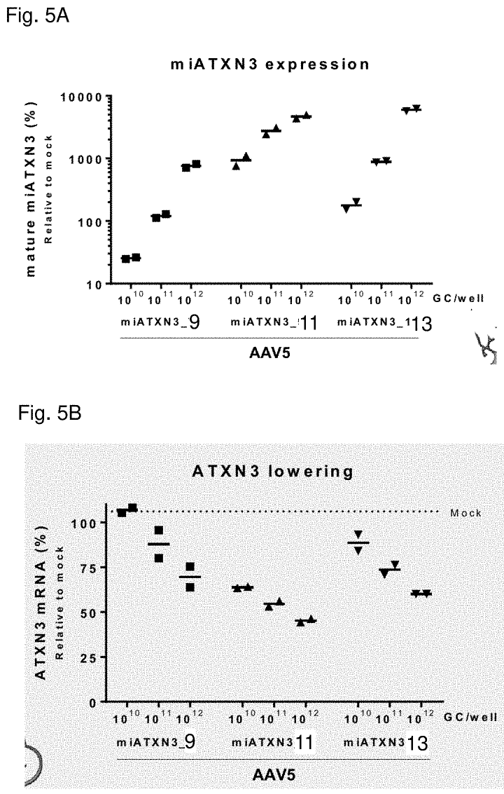

The expression cassettes were incorporated in an AAV viral vector genome. Subsequently, recombinant viral vectors based on the AAV5 serotype were produced using the insect cell baculovirus based manufacturing and standard down-stream processing utilizing chromatography methods, including affinity chromatography and filtration methods (Lubelski et al. Bioprocessing Journal, 2015, Weihong Qu et al., Curr Pharm Biotechnol, 2014, AVB sepharose high performance, GE Healthcare Life Sciences, ref. 28-9207-54 AB). These viral vectors were subsequently used to transduce iPSC (induced pluripotent stem cells) derived frontal brain-like neurons by dual inhibition of SMAD signaling as described (Chambers S M, Nat Biotechnol, 2009). An increasing dosage of AAV vector (10exp11, 10exp 12, 10exp 13, genomic copies as determined with qPCR) was added to each well comprising 3*10⁵ neuronal cells. A clear dose response was observed when targeting SEQ ID NOs. 9, 11 and 13, both for miRNA expression levels as well as knock down of ATXN3 mRNA (FIGS. 5a and 5b, respectively). A reduction of ATXN3 mRNA of about 65% was observed. In addition to assessing knock down of the endogenous ATXN3 gene expression, the processing of the miRNA scaffolds that were expressed in these iPSC derived neurons was assessed using high throughput small RNA sequencing. The miRNAs targeting SEQ ID NO. 9, 11 and 13 were highly expressed in the transduced iPSC neurons. Of the total miRNA counts, 0.003% to 5.7% were aligned to the mature sequences targeting ATXN3. The sequences listed in the tables 6-8 below show that the most abundant reads as determined by small RNA sequencing from transduced neuronal cultures. Note that the sequences listed in tables 6-8 represent DNA sequences, whereas these sequences represent RNA sequences derived from miRNA scaffolds (such as depicted i.a. in FIGS. 2a and 8) as processed by the cell (i.e. a T is a U).

TABLE 6
Sequences derived from miR451 scaffold targeting SEQ ID NO. 9

| SEQ ID | | | | |
|---|---|---|---|---|
| NO. | SEQUENCE (5′-NNN-3′) | length | % reads | SD |
| 30 | TTTCTAACTGTAAACCAGTGTTCTG | 25 | 31.5 | 0.1 |
| 31 | TTTCTAACTGTAAACCAGTGTTCT | 24 | 21.0 | 0.1 |
| 32 | TTTCTAACTGTAAACCAGTGTTCTGGTTT | 29 | 13.0 | 1.0 |
| 33 | TTTCTAACTGTAAACCAGTGTTCTGGTT | 28 | 8.0 | 0.3 |
| 34 | TTTCTAACTGTAAACCAGTGTTCTT | 26 | 5.5 | 0.2 |
| | | total: | 79.0 | 1.1 |

TABLE 7
Sequences derived from miR451 scaffold targeting SEQ ID NO. 11

| SEQ ID | | | % | |
|---|---|---|---|---|
| NO. | SEQUENCE (5′-NNN-3′) | length | reads | SD |
| 35 | TCTGGAACTACCTTGCATACTT | 22 | 53.0 | 2.6 |
| 36 | TCTGGAACTACCTTGCATACTTAT | 24 | 18.0 | 1.5 |
| 37 | TCTGGAACTACCTTGCATACTTA | 23 | 11.0 | 0.1 |
| 38 | TCTGGAACTACCTTGCATACT | 21 | 6.1 | 0.8 |
| 39 | TCTGGAACTACCTTGCATACTTATGCAAGG | 30 | 2.9 | 1.0 |
| | | | 91.0 | 0.9 |

TABLE 7
Sequences derived from miR451 scaffold targeting SEQ ID NO. 13

| SEQ ID | | | | |
|---|---|---|---|---|
| NO. | SEQUENCE (5′-NNN-3′) | length | % reads | SD |
| 40 | TTCAAAGTAGGCTTCTCGTCTCCG | 24 | 28.7 | 1.2 |
| 41 | TTCAAAGTAGGCTTCTCGTCTCC | 23 | 13.0 | 0.3 |
| 42 | TTCAAAGTAGGCTTCTCGTCTCCGA | 25 | 7.2 | 0.2 |
| 43 | TTCAAAGTAGGCTTCTCGTCTCCGAG | 26 | 7.2 | 0.2 |
| 44 | TTCAAAGTAGGCTTCTCGTCTCCGAGT | 27 | 7.0 | 1.5 |
| 45 | TTCAAAGTAGGCTTCTCGTCT | 21 | 6.7 | 0.5 |
| 46 | TTCAAAGTAGGCTTCTCGTCTC | 22 | 5.5 | 0.3 |
| 47 | TTCAAAGTAGGCTTCTCGTCTCCGAGA | 27 | 5.5 | 0.9 |
| 48 | TTCAAAGTAGGCTTCTCGTCTCCGT | 25 | 3.9 | 0.4 |
| | | total: | 84.6 | 0.3 |

It is noted that the RNA molecules as processed by the RNAi machinery of the cell produce RNA molecules that are in the range of 21-30 nucleotides in length. The RNA molecules that extend beyond 22 nucleotides include at most 8 nucleotides that are derived from the sequence representing the second RNA sequence. It is further noted that for the RNA molecules that target SEQ ID NO. 11 that of the four most dominant species, 3 are 100% complementary to the target sequence (i.e. SEQ ID NO. 35, 37 and 38) whereas SEQ ID NO.36 has one mismatch at the 5′-end of the RNA sequence, and that the four most dominant species have a length ranging from 21-24 nucleotides, representing up to 90% of the RNA species produced from the scaffold. Based on processing, a preferred target RNA selected may thus be SEQ ID NO.11, for which preferably a miRNA scaffold based on miR451 may be useful having a sequence such as SEQ ID NO.24 or SEQ ID NO. 28, or as encoded by SEQ ID NO. 49.

### In Vivo Lowering of SCA3

In order to test for in vivo activity of the most preferred RNA target sequences, in a knock-in mouse model, AAV based gene delivery was tested. The mouse model used was a novel F512 SCA3 knock-in mouse model. In this mouse model, a CAG expansion was inserted into the endogenous murine Atxn3 gene. This model was generated using Zinc Finger technology by cutting the murine (CAG)6 and subsequent homologous recombination with a (CAACAGCAG)48 donor vector with interrupted repeat. The F512 SCA3 knock-in mouse model was characterized to express a mutant ataxin-3 protein with a 233 glutamine repeat. This model contains target sequences representing at least the human sequences SEQ ID NO. 9, 11 and 13. It is noted that the endogenous target sequence corresponding to SEQ ID NO.11 in this model contains a mismatch with the first RNA sequence SEQ ID NO. 16, said mismatch representing an A to C at position 1 of SEQ ID NO.11.

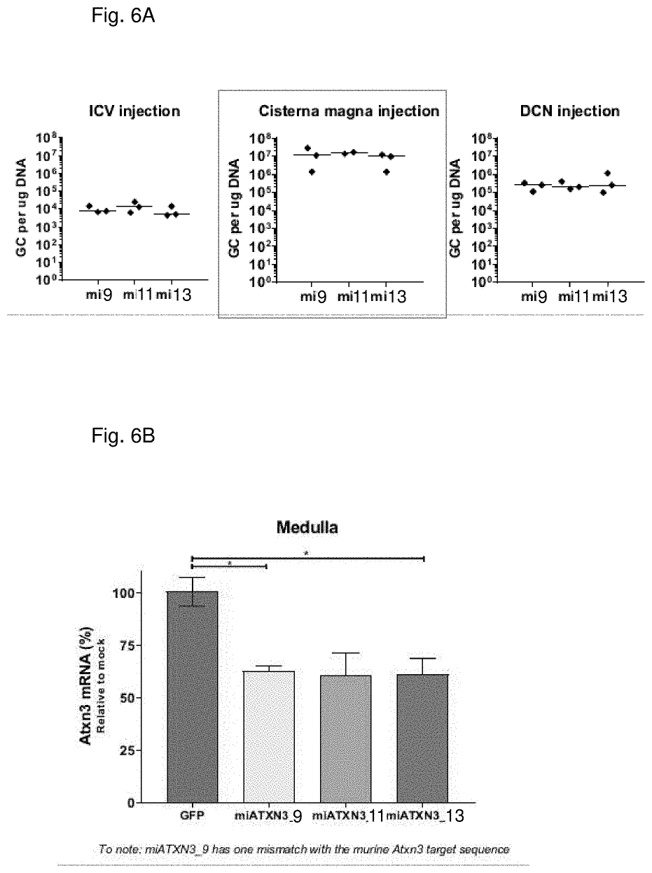

Viral vector was injected in the deep-cerebellar nuclei, ICV or intra cisterna magna of F512 SCA3 knock-in mice (FIG. 6). Three animals per RNA molecule that target SEQ ID NO. 9, 11 and 13 were used. After six weeks in-life, animals were sacrificed, and brains dissected. The gc copy number was determined, as well as the amount of ATXN3 mRNA in the cerebellum, brain stem and cortex. Consistently, transduction levels were similar, and ATXN3 lowering was consistent between different groups as well. It is noted that because the putative guide strand that targets the human sequence SEQ ID NO.11 that is produced in the mouse has a mismatch with its mouse ATXN3, ATXN3 lowering observed can be underestimated, as having full complementarity is expected to reduce ataxin-3 lowering further. Hence, based on these results, it is expected that targeting SEQ ID NO. 9, 10, 11 and 13 in humans will result in sufficient lowering of ATXN3, whereas it is expected that targeting SEQ ID NO. 11 in humans will result in the strongest lowering.

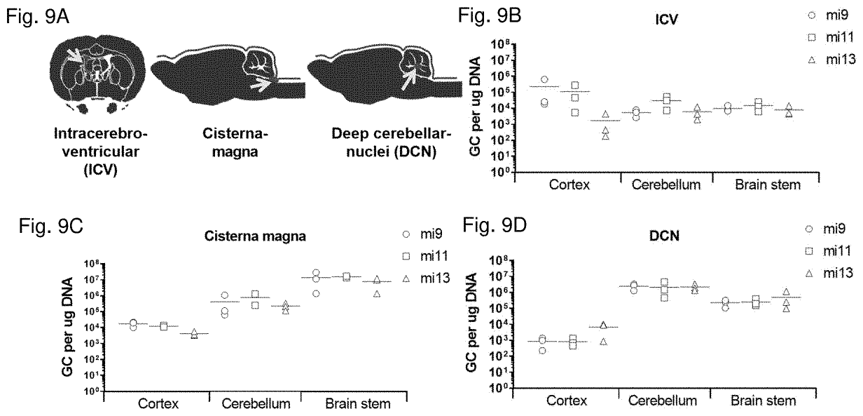

Further results of in vivo administration of AAV targeting SEQ ID NO. 9, 11 and 13 are presented in FIG. 9. As said, AAV was injected in F512 SCA3 mice via 3 different injection routes: ICV, intra cisterna magna, or in the DCN (FIG. 9A). Injections were performed with viral vectors comprising RNA that target SEQ ID NO. 9, 11 or 13 (i.e., AAV5-miATXN3_9, AAV5-miATXN3_11, AAV5-miATXN3_13) and AAV5-GFP was taken along as control. The amount of gc detected per genomic DNA was determined for each administration route in the cortex, cerebellum and brain stem. ICV administration resulted in a relative low vector copy distribution to all three analyzed brain regions. Compared to the cerebellum and brain stem, a higher transduction was observed in the cortex (FIG. 9B). Administration into the cisterna magna resulted in low transduction of the cortex but strong transduction of the brain stem and cerebellum (FIG. 9C). The highest transduction was detected in the brain stem with up to 2.9×10⁷ genome copies (gc)/g tissue DNA. Direct injection into the DCN also resulted in relatively high transduction of the cerebellum and the brain stem. Compared to cisterna magna administration, DCN injection resulted in better transduction of the cerebellum and less transduction of the brain stem (FIG. 9D). Based on the current observations, all three administration routes resulted in transduction of the brain but administration into the cisterna magna resulted in the highest combined transduction of both cerebellum and brain stem of mice. In patients, cerebellum and brain stem are the main affected regions.

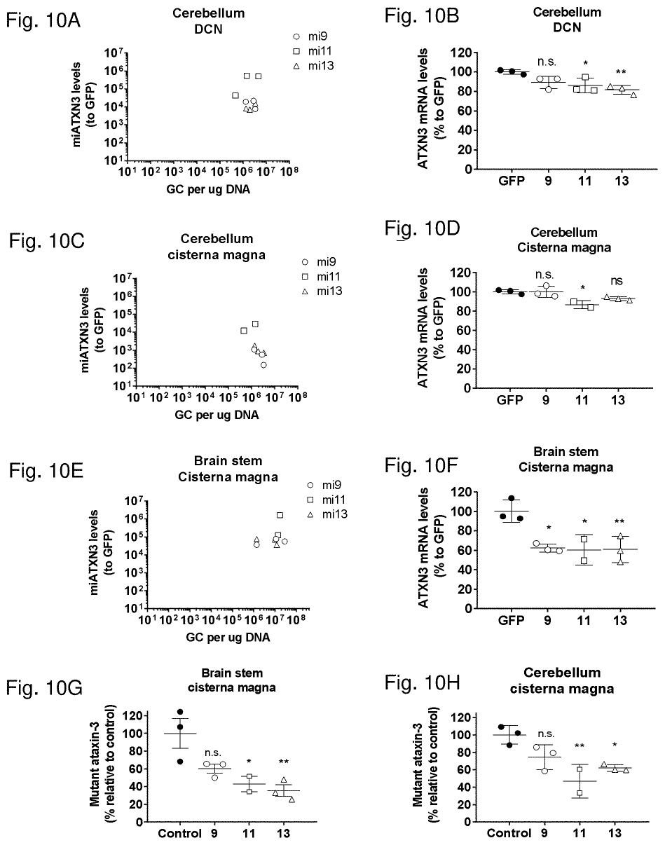

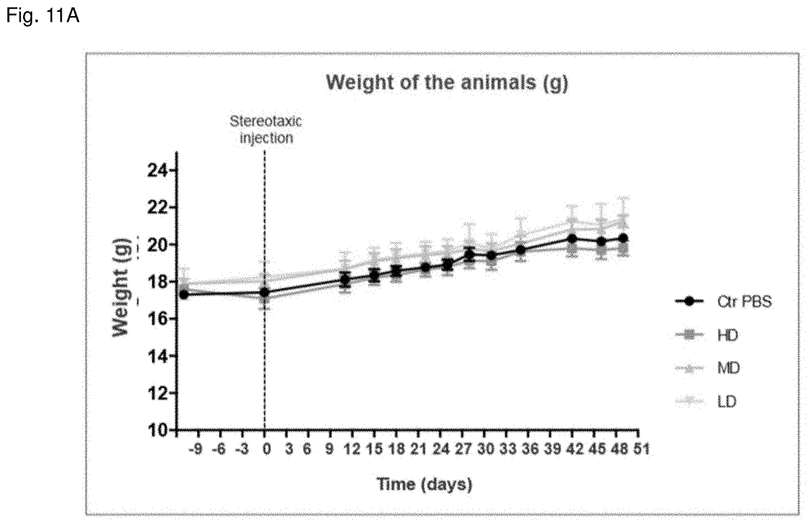

The miATXN3 expression and silencing of mutant ataxin-3 in F512 mice was further analysed. Direct injection into the DCN showed highest expression of mature miATXN3 in the cerebellum (FIG. 10A). miATXN3_11 showed the highest microRNA expression. The microRNA expression correlated well with a (˜15-20%) significant reduction of ATXN3 mRNA by miATXN3_11 and miATXN3_13 in the cerebellum (FIG. 10B). Administration to the cisterna magna resulted into lower mature microRNA expression in the cerebellum as compared to DCN injection (FIG. 10C). Nevertheless, miATXN3_11 was the best expressed and resulted in significant lowering (˜15%) of ATXN3 mRNA in the cerebellum (FIG. 10D). The highest microRNA expression and silencing efficacy from all three delivery routes was observed in the brain stem after administration in the cisterna magna (FIG. 10E-F). Expression of the miATXN3 candidates were high in the brain stem and all led to a strong reduction of ATXN3 mRNA of about 40%. Both AAV5-miATXN3_11 and AAV5-miATXN3_13 had comparable efficacies in the brain stem. AAV5_miATXN3_11 to the cisterna magna resulted in ATXN3 mRNA reduction in both cerebellum and brain stem, which are the main areas affected in SCA3 patients.

### In Vivo Testing of Constructs in Transgenic Mice Carrying Pathological Alleles of the Human SCA3 Locus

Transgenic (tg) mice carrying pathological alleles of the human MJD1 locus have been described (Cemal et al., Human Molecular Genetics 2002 (11) 1075-1094). These tg mice contain pathological alleles with polyglutamine tract lengths of 64, 67, 72, 76 and 84 repeats. As a control, tg mice containing the wild type with 15 repeats, were generated. It has been shown that tg mice with these expanded alleles demonstrate a mild and slowly progressive cerebellar deficit. Disease severity in this model increased with the level of expression of the expanded protein and the size of the repeat. Tg mice with an expanded repeat at the high end of the human disease range, CAG₈₄ (Q84, Tg(ATXN3*)84.2Cce/Tg(ATXN3*)84.2Cce) recapitulate several key pathological hallmarks of SCA3 and display early onset, readily quantifiable motor phenotype. In contrast, tg mice carrying a normal length CAG repeat (wild-type CAG₁₅, Q15) appeared completely normal (Rodriguez-Lebron et al., Mol Ther. 2013 (21)1909-1918; Costa et al., Mol. Ther. 2013 (21)1898-1908).

In subsequent experiments, the most preferred RNA target sequences as described above are tested, using AAV based gene delivery, in the above described transgenic mouse model for human SCA3 disease. In the present study, homozygous Q84/Q84 mice are studied with a focus on selective reduction of human ATXN3 expression, improved motor function and prolonged survival after AAV-based delivery of miRNA's targeting the region 5′ of the CAG repeat region of ATXN3.

AAV-miATXN3 vectors are injected into approximately two months old Tg(ATXN3*)84.2Cce/Tg(ATXN3*)84.2Cce homozygous transgenic SCA3 mice. One cohort is used as control arm. The route of injection is in the cisterna magna. During the in-life phase, body weight is monitored. Beam-walk and Open Field testing is performed pre-dosing and monthly post-injection to explore potential functional improvements. Four to seven months post-injection molecular analysis is performed to assess biodistribution, biological activity, and therapeutic efficacy of the AAV-miATXN3s. Key expected findings are lowering of human mutant ataxin-3 with subsequent mitigation of mutant ataxin-3 aggregation, resulting in halting of neurodegeneration and functional improvement, being improvement of motor dysfuctioning.

In a second study, Tg(ATXN3*)84.2Cce/Tg(ATXN3*)84.2Cce Homozygous transgenic SCA3 mice are injected as described above and are used for survival analysis. Key findings are expected to be increased median survival of the homozygous SCA3 mice upon one-time AAV-miATXN3 treatment.

In Vivo Testing of Constructs in Mice that Overexpress Mutant Ataxin-3 Upon Injection of Lentiviral Vectors Encoding Full-Length Human Mutant Ataxin-3.

In further experiments, the most preferred RNA target sequences are tested, using AAV based gene delivery, in another mouse model for human SCA3 disease as described in Nobrega et al., Cerebellum 2013 (12) 441-455. Briefly, lentiviral vector-based expression of human mutant ataxin-3 in the mouse striatum has been shown to induce localized neuropathology. Such mice provide for an efficient model to evaluate the therapeutic potential of our RNAi approach. AAV-miATXN3 viruses are bilaterally co-injected with the lentiviral vector, into approximately 2 months old mice in a low, medium and high AAV dosage (total of three cohorts). One other cohorts is injected with the lentiviral vectors and controls. The group sizes are 8 mice per group. The route and region of injection is a stereotaxic bilateral striatal injection. Mutant ataxin-3 levels, as well as AAV genome copies are determined. Likewise, mutant ataxin-3 aggregates and area of darpp-32 loss of immunoreactivity is quantified. Key expected findings are mitigation of mutant ataxin-3 aggregation and prevention of neurodegeneration.

### Striatal Viral Injections in Mice

Injections were performed as described previously (Goncalves et al., (2013) Ann Neurol, 73(5), 655-666). In brief, mice of 2 months of age were anesthetized with avertin (12 μL/g, i.p.), and a mixture of lentiviral vectors (encoding mutant ataxin-3 (atx3-72Q)) and AAV5-miATXN3_11 were stereotaxically injected into the striatum. Coordinates: anteroposterior: +0.6 mm; lateral: ±1.8 mm; ventral: −3.3 mm; tooth bar: 0. These coordinates correspond to the internal capsule, a large fiber tract passing through the middle of the striatum dividing both dorso-ventral and medial-lateral structures. Mice received 2 μL injections consisting of 1 μL of lentivirus (200′000 ng of p24/mL) and 1 μL AAV5-miATXN3 in each hemisphere, in total 2×10⁹ to 5×10¹⁰ genome copies per mouse. 7 Weeks following injection, mice were killed for immunohistochemical analysis of morphological and neurochemical changes, as well as ataxin-3 levels in the striatum.

### Tissue Preparation

After an overdose of ketamine/xilazine, mice were intracardiacally perfused with cold PBS 1×. The brains were then removed and left- and right hemispheres were divided. The right hemisphere was post-fixed in 4% paraformaldehyde for 72 h at 4° C. and cryoprotected by incubation in 25% sucrose/PBS1× for 48 h at 4° C. In the left hemisphere, the striatum was dissected and kept at −80° C. for RNA/DNA/protein extraction. For each animal, 120 coronal sections of 25% were cut throughout the right brain hemisphere using a cryostat (LEICA CM3050S, Germany) at −20° C. Individual sections were then collected and stored in 48 well plates, as free-floating sections in PBS 1× supplemented with 0.05% sodium azide at 4° C.

Purification of Total RNA and Protein from Mouse Striata

Left part of the striatum was homogenized with QIAshredder (QIAGEN) columns. After homogenization, RNA, DNA and protein were isolated using All Prep DNA/RNA/Protein Kit (QIAGEN) according to the manufacturer's instructions. The initial volume of buffer RLT added to the striatum was 350 μL. Total amount of RNA was quantified using a Nanodrop 2000 Spectrophotometer (Thermo Scientific) and the purity was evaluated by measuring the ratio of OD at 260 and 280 nm. Protein was dissolved in a solution of 8M Urea in 100 mM Tris-HCl pH8 1% SDS and sonicated at 50 mA with 1 pulse of 3 s. Total protein extracts were stored at −80° C.

cDNA Synthesis and Quantitative Real-Time PCR (qPCR)

Firstly, in order to avoid genomic DNA contamination in RNA preps, DNase treatment was prior performed using Qiagen RNase-Free DNase Set (Qiagen, Hilden, Germany), according to the manufacturer's instructions. cDNA was then obtained by conversion of total decontaminated RNA using the iScript Select cDNA Synthesis Kit (Bio-Rad, Hercules, USA) according to the manufacturer's instructions. After reverse transcriptase reaction, the mixtures were stored at −20° C. Quantitative real-time PCR (qPCR) was performed using the SsoAdvanced SYBR Green Supermix (BioRad, Hercules, USA), according to the manufacturer's instructions. Briefly, the qPCR reaction was performed in a total volume of 20 μl, containing 10 μL of this mix, 10 ng of DNA template and 500 nM of validated specific primers for human ataxin-3, mouse ataxin-3 and mouse hypoxanthine guanine phosphoribosyl transferase (HPRT). The qPCR protocol was initiated by a denaturation program (95° C. for 30 seconds), followed by 40 cycles of two steps: denaturation at 95° C. for 5 seconds and annealing/extension at 56° C. for 10 seconds. The cycle threshold values (Ct) were determined automatically by the StepOnePlus software (Life technologies, USA). For each gene, standard curves were obtained, and quantitative PCR efficiency was determined by the software. The mRNA relative quantification with respect to control samples was determined by the Pfaffl method (Pfaff et al. (2001) NAR, May 1, 29(9): e45).

### Western Blotting

BCA protein assay kit (Thermo Fisher Scientific) was used to determine protein concentration. Seventy micrograms of striatum protein extracts were resolved on sodium dodecyl sulfate-polyacrylamide gels (4% stacking and 10% running). Proteins were then transferred onto a polyvinylidene difluoride membrane (Millipore), blocked with 5% non-fat milk powder dissolved in 0.1% Tween 20 in Tris-buffered saline for 1 hour at room temperature. Membranes were then incubated overnight at 4° C. with primary antibodies: mouse anti-1H9 (1:1000, Millipore) and mouse anti-βactin (1:5000). The correspondent alkaline phosphatase-linked goat anti-mouse secondary antibody was incubated for 2 hours at room temperature. Bands were detected after incubation with Enhanced Chemifluorescence Substrate (GE Healthcare) and visualized in chemiluminescent imaging (ChemiDoc™ Touch Imaging System, Bio-Rad Laboratories). Semi-quantitative analysis was carried out based on the bands of scanned membranes using Image J (National Institutes of Health) and normalized with respect to the amount of β-actin loaded in the corresponding lane of the same gel.

### Immunohistochemistry

For each animal, 16 and 12 coronal sections with an intersection distance of 200 m were selected for DARPP32 and 1H9 (ataxin-3) staining, respectively. The procedure started with endogenous peroxidase inhibition by incubating the sections in PBS containing 0.1% Phenylhydrazine (Merck, USA), for 30 minutes at 37° C. Subsequently, tissue blocking and permeabilization were performed in 0.1% Triton X-100 with 10% NGS (normal goat serum, Gibco) prepared in PBS, for 1 hour at room temperature. Sections were then incubated overnight at 4° C. with the primary antibodies Rabbit Anti-DARPP32 (Millipore) and Chicken Anti-1H9 (HenBiotech), previously prepared on blocking solution at the appropriate dilution (1:2000). After three washings, brain slices were incubated in anti-rabbit or anti-chicken biotinylated secondary antibody (Vector Laboratories) diluted in blocking solution (1:250), at room temperature for 2 h. Subsequently, free-floating sections were rinsed and treated with Vectastain ABC kit (Vector Laboratories) during 30 minutes at room temperature, inducing the formation of Avidin/Biotinylated peroxidase complexes. The signal was then developed by incubating slices with the peroxidase substrate: 3,3′-diaminobenzidine tetrahydrochloride (DAB Substrate Kit, Vector Laboratories). The reaction was stopped after achieving optimal staining, by washing the sections in PBS. Brain sections were subsequently mounted on gelatin-coated slides, dehydrated in an ascending ethanol series (75, 95 and 100%), cleared with xylene and finally coverslipped using Eukitt mounting medium (Sigma-Aldrich).

### Evaluation of the Volume of DARPP-32 Depleted Region

Images of coronal brain sections subjected to immunohistochemistry were obtained in Zeiss Axio Scan.Z1 microscope. Whole-brain images were acquired with a Plan Apochromat 20×/0.8 objective. The extent of DARPP-32 loss in the striatum was analyzed by digitizing the stained-sections (25 μm thickness sections at 200 μm intervals) to obtain complete rostrocaudal sampling of the striatum. To calculate the DARPP-32 loss, sections were imaged using the tiles feature of the Zen software (Zeiss). The depleted area of the striatum was estimated using the following formula: Volume=d (a1+a2+a3+ . . . ), where d is the distance between serial sections (200 μm) and a1, a2, a3 are DARPP-32-depleted areas for individual serial sections.

### Quantitative Analysis of Ataxin-3 Aggregates (1H9 Staining)

Images of coronal brain sections subjected to immunohistochemistry were obtained in Zeiss Axio Scan.Z1 microscope (25₁ m thickness sections at 200 μm intervals). Whole-brain images were acquired with a Plan Apochromat 20×/0.8 objective. Striatal stained-sections were selected following the same criteria for all animals: i.e. the section with higher DARPP-32-depleted area in the control group was firstly identified and its anatomical position was considered the center for the selection of 10 sections for further 1H9-positive inclusions quantification. All striatal 1H9-positive inclusions were counted in the selected sections using an automatic image-analysis software (Qupath).

### Statistical Analysis

Statistical analysis was performed using Prism GraphPad software. Data are presented as mean±standard error of mean (SEM) and outliers were removed according to Grubb's test (alpha=0.05). Oneway ANOVA test was used for multiple comparisons. Correlations between parameters were determined according to Pearson's correlation coefficient. Significance was determined according to the following criteria: p>0.05=not significant (ns); *p<0.05, **p<0.01 ***p<0.001 and ****p<0.0001.

### Results

### AAV5-miATXN3 Induces Strong Ataxin-3 Knockdown in a Lentiviral SCA3 Mouse Model

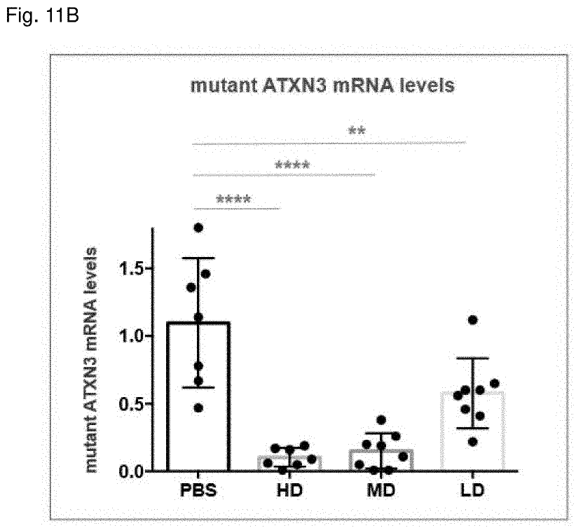

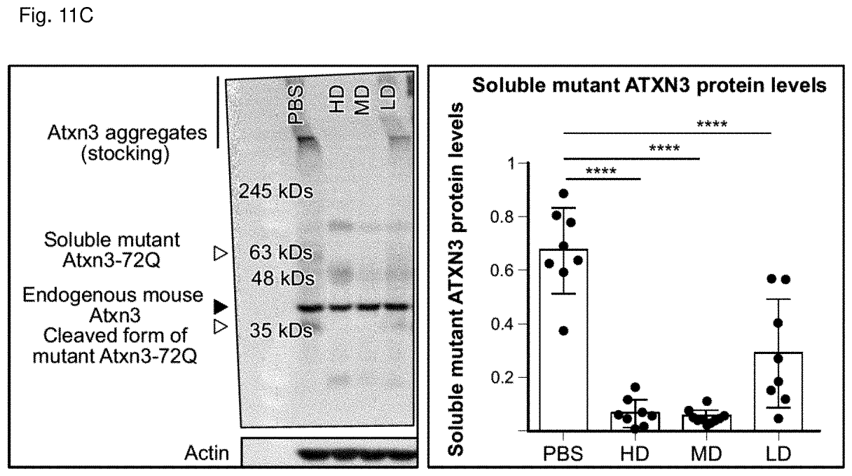

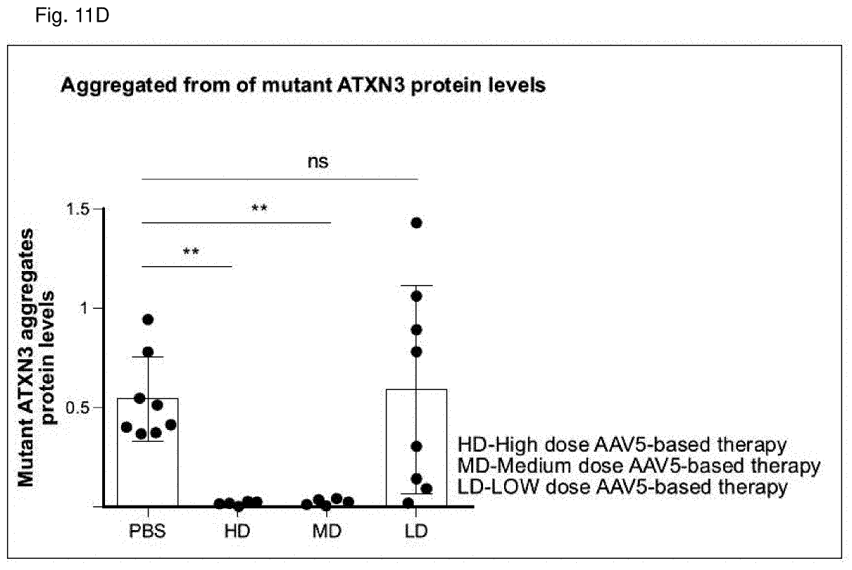

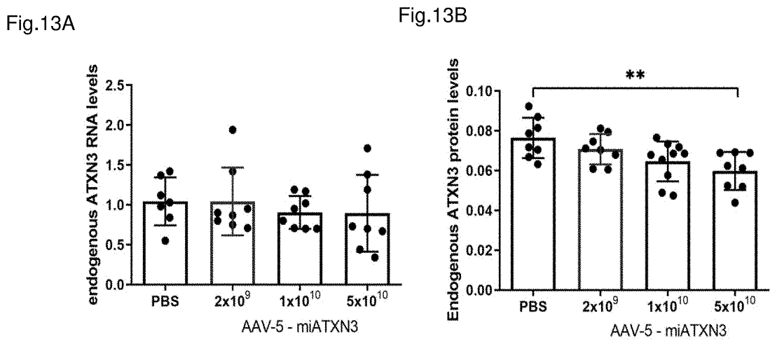

To confirm in vivo potency of AAV5 delivered miATXN3, bilateral striatal injections were performed in mice. AAV5-miATXN3_11 was co-injected with a lentiviral vector encoding mutant ataxin-3 (72Q). This lentiviral SCA3 mouse model presents strong expression of mutant ataxin-3 (72Q) throughout the striatum, resulting in several molecular hallmarks of disease in this brain structure (Goncalves et al., (2013) Ann Neurol, 73(5), 655-666). Mice were followed for 7 weeks after injection, and no effect of the AAV on bodyweight was observed during this period FIG. 11A. The right striatum of the mice was used for molecular analysis, where expression of the mutant ataxin-3 protein was confirmed through qPCR in the PBS treated control group. In contrast, a robust knockdown of mutant ataxin-3 mRNA was observed in the miATXN3 treated animals. The low dose (2×10⁹ gc) of AAV5 resulted in approximately 50% ATXN3 mRNA knockdown, whereas the mid (1×10¹⁰ gc) and high dose (2×10¹⁰ gc) almost completely abolished ATXN3 expression (FIG. 11B). Of note, endogenous mouse ATNX3 RNA was not affected by miATXN3 treatment (FIG. 13), despite carrying only one mismatch in the target sequence.

Similar to SCA3 patients, the mouse model used here presents with both soluble and insoluble forms of the mutant ataxin-3 protein. Through western blot analysis, these different states of the ataxin-3 protein can be investigated, as the high molecular weight aggregates do not migrate into the separating gel. As predicted by the mRNA results, a dose dependent reduction in both the soluble and insoluble ataxin-3 protein was observed. Notably, the putatively toxic ataxin-3 aggregates were completely abolished by miATXN3 treatment (FIGS. 11C and D). Additionally, soluble ataxin-3 protein levels in striatum closely mirrored mRNA levels, with low dose treatment resulting in approximately 50% reduction and the high miATXN3 dose reducing ataxin-3 protein levels by about 90%. In contrast to the mRNA results, a slight reduction of the endogenous murine ataxin-3 protein was observed after the high dose miATXN3 treatment. Together, these results suggest a strong potency of miATXN3 against the ATXN3 gene, with only mild off-target efficacy.

### Reduction in Ataxin-3 Inclusions

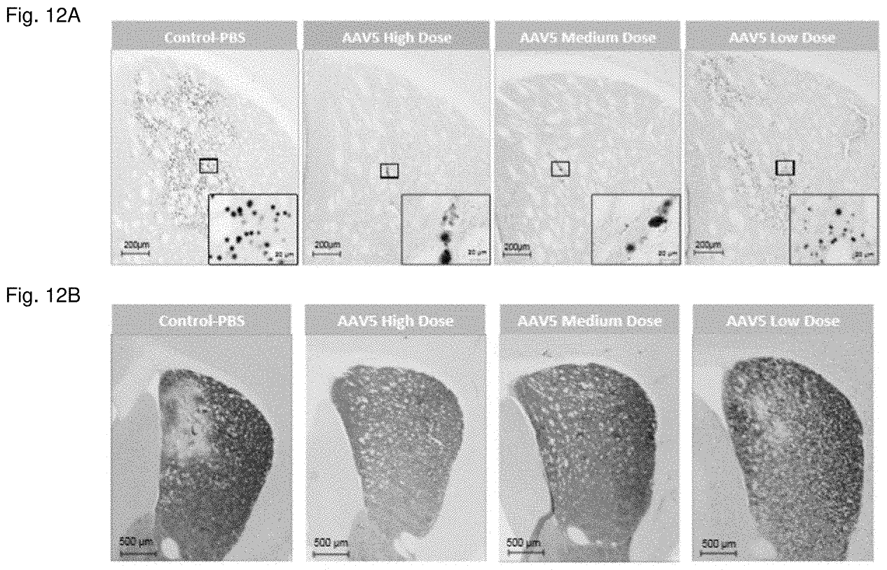

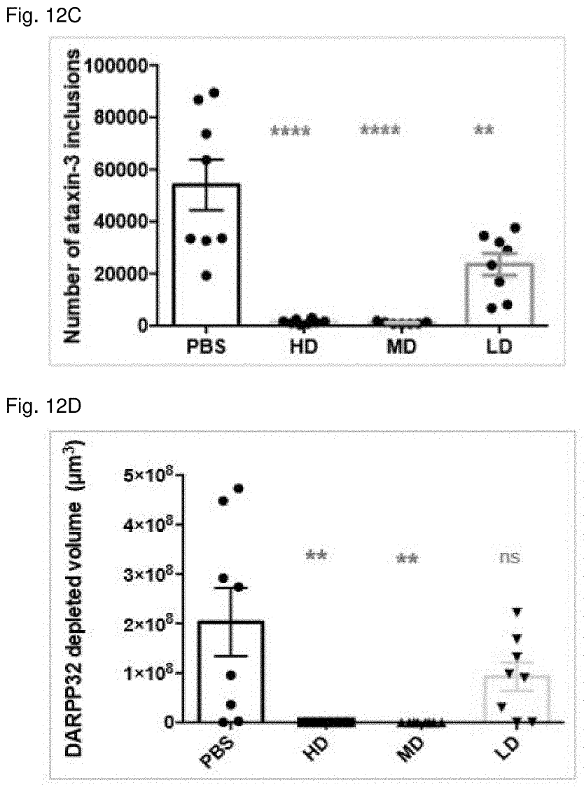

The lentiviral SCA3 mouse model used here also develops several histological features of SCA3 as a result of continuous ataxin-3 71Q expression (Goncalves et al., (2013) Ann Neurol, 73(5), 655-666). Of particular interest are the hallmark ataxin-3 inclusions (Paulson et al., (1997) Neuron, 19(2), 333-344; Schmidt et al., (1998) Brain Pathol, 8(4), 669-679), that form in the area transduced with the expression cassette. These protein inclusions only occur with longer repeat lengths and correlate with disease progression in these mice. Similar to shown with the western blot analysis, histological examination of the SCA3 mouse brain revealed a very strong reduction in the ataxin-3 inclusion burden throughout the striatum (FIGS. 12A and C). Low dose miATXN3 treatment reduced the number of ataxin-3 inclusions by about 50%, whilst almost no nuclear inclusions could be detected in mid and high dose miATXN3. Interestingly, the inclusion count in mice treated with the low dose miATXN3 directly correlated with the 50% reduction in mutant ataxin-3 mRNA and total ataxin-3 protein levels in this treatment group. This suggests that the total number of inclusions is closely affected by expression levels of the mutant protein. It must also be mentioned that the ataxin-3 expression level in the lentiviral mouse model used here is at least 4× higher than the endogenous ataxin-3 (Alves et al., 2008, Hum Mol Genet, 17(14), 2071-2083; Goncalves et al., (2013) Ann Neurol, 73(5), 655-666). Hence, at endogenous expression levels a less substantial knockdown than reported here may be sufficient to prevent onset of the nuclear ataxin-3 inclusions.

### Rescue of Neuronal Dysfunction

Similar to the other polyglutamine proteins, mutant ataxin-3 is known to induce cellular stress and neuronal dysfunction over time (Evers et al., (2014), Mol Neurobiol, 49(3), 1513-1531; Weber et al., (2014) Biomed Res Int, 2014, 701758). We performed immunostainings on the striatal dopaminergic marker darpp-32 to assess the extent of neuronal dysfunction in the SCA3 mice. In line with previous reports (Alves et al., 2008, Hum Mol Genet, 17(14), 2071-2083; Goncalves et al., (2013) Ann Neurol, 73(5), 655-666), the PBS treated SCA3 mice presented with a darpp-32 depleted region in the striatum of about 2*10⁸ μm³ on average (FIGS. 12B and 12D). Low dose miATXN3 treatment resulted in an average lesion size that was on average half the size of PBS treated animals, though this did not reach statistical significance (p=0.19). In contrast, animals treated with mid and high dose miATXN3 showed remarkable improvement in this phenotype, as all but one animal did not present with an any observable darpp-32 depleted area. In early stage of polyglutamine disease such as reported here, darpp-32 downregulation likely represents onset of neuronal dysfunction, such as synaptic signaling deficits (van Dellen et al., (2000) Neuroreport, 11(17), 3751-3757). Moreover, darpp-32 is involved in regulation of electrophysiological and transcriptional responses (Svenningsson et al., (2004) Annu Rev Pharmacol Toxicol, 44, 269-296) further underlining importance of retaining expression of this protein to maintain neuronal health.

### In Vivo Testing of Constructs in NHP

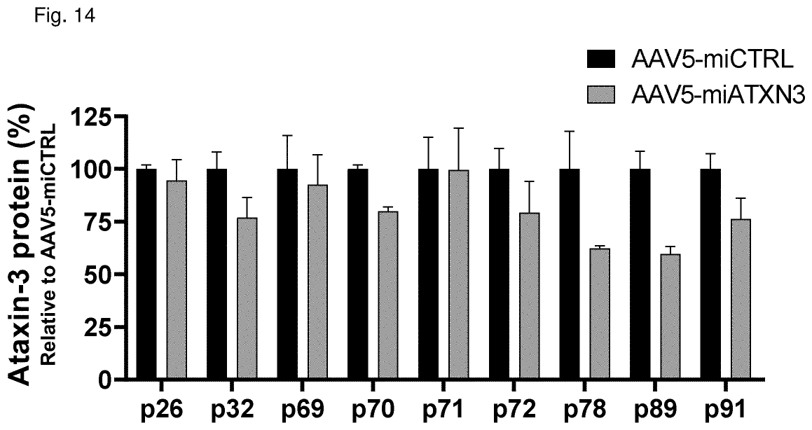

Cynomolgous macaques were injected with approximately 1×10¹³ to 1×10¹⁴ genome copies per kg AAV5-miATXN3_11 into the cisterna magna and/or intrathecal space. In total 3 Cynomolgous macaques were injected per dose of AAV5-miATXN3_11 and 3 with a—for SCA3—control AAV-miRNA. After 1 to 2 months in-life the animals were sacrificed, and molecular analysis performed on brain punches and peripheral organs to assess vector genome copies and ataxin-3 RNA and protein lowering. The key findings were ataxin-3 lowering up to 40% after one-time intra-CSF administration without acute toxicology or miATXN3-related off-target effects (FIG. 14).

With our miATXN3 candidates we have shown a dose-dependent lowering of mutant ataxin-3 in SCA3 knock-in mice and prevention of toxic ataxin-3 aggregation in LV-SCA3 mice. This lowering resulted in complete prevention of neuropathology in LV-SCA3 mouse brain with both medium and high dose of miATXN3. One-time intrathecal administration of a medium dose of AAV5-miATXN3 in cynomolgous monkeys resulted in favorable transduction, miATXN3 expression and subsequent up to 40% endogenous ataxin-3 protein lowering in the deep cerebellar nuclei, which is the brain area most affected by SCA3. To our knowledge, this is the first proof-of-concept of RNAi-mediated ataxin-3 lowering in a large animal model. These results in SCA3 rodents and large animals show the disease-modifying potential of AAV-based miATXN3.

### Embodiments

1. An expression cassette encoding a double stranded RNA comprising a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human ATXN3 gene.
2. An expression cassette according to embodiment 1, wherein said target RNA sequence is comprised in the region 5′ to the RNA sequence encoded by the sequence corresponding with nucleotides 942-1060 of SEQ ID NO. 2 of the human ATXN3 gene.
3. An expression cassette according to embodiment 2, wherein said target RNA sequence is comprised in the RNA sequence encoded by the region 390-456 of SEQ ID NO.2 and sequences 3′ therefrom.
4. An expression cassette according to any one of embodiments 2-3, wherein said target RNA sequence is selected from the group consisting of SEQ ID NOs. 3-13, more preferably from the group consisting of SEQ ID NOs. 9-13.
5. An expression cassette according to embodiment 1, wherein said target RNA sequence is SEQ ID NO. 11.
6. An expression cassette according to any one of embodiments 1-5, wherein said first and second RNA sequence are comprised in a pre-miRNA scaffold, a pri-miRNA scaffold or a shRNA.
7. An expression cassette according to embodiment 6, wherein said pre-miRNA scaffold or said pri-miRNA scaffold is from miR451.
8. An expression cassette according to any one of embodiments 1-7, wherein said first RNA sequence is comprised in a guide sequence.
9. An expression cassette according to any one of embodiments 1-8 wherein said first RNA sequence and said second RNA sequence, when expressed in a cell, are processed by the cell to produce a guide sequence comprising the first RNA sequence.
10. An expression cassette according to any one of embodiments 1-9 wherein the first RNA sequence is selected from the group consisting of SEQ ID NOs. 14-17.
11. An expression cassette according to any one of embodiments 1-10 wherein the second RNA sequence is selected from the group consisting of SEQ ID NOs. 18-21.
12. An expression cassette according to embodiment 11 wherein the first RNA sequence and second RNA sequence are selected from the group consisting of the combinations of SEQ ID NOs. 14 and 18; SEQ ID NOs. 15 and 19; SEQ ID NOs. 16 and 20; SEQ ID NOs. 17 and 21.
13. An expression cassette according to embodiment 12, wherein said encoded RNA comprises an RNA sequence selected from the group consisting of SEQ ID NOs. 22-29.
14. An expression cassette according to any one of embodiments 1-13, wherein the expression cassette comprises a PGK promoter, a CMV promoter, a neuron-specific promoter, a astrocyte-specific promoter or a CBA promoter operably linked to said nucleic acid sequence encoding said first RNA sequence and said second RNA sequence.
15. An expression cassette according to any one of embodiments 1-13, wherein the expression cassette comprises an inducible or repressible promoter, operably linked to said nucleic acid sequence encoding said first RNA sequence and said second RNA sequence.
16. A gene therapy vector comprising the expression cassette according to any one of embodiments 1-15, wherein said gene therapy vector preferably is an AAV vector.
17. A gene therapy vector according to embodiment 16, or an expression cassette according to any one of embodiments 1-15, for use in a medical treatment.
18. A use in accordance with embodiment 17, wherein said use is in a medical treatment of SCA3/MJD.
19. A use in accordance with embodiment 17 or embodiment 18, wherein said use comprises at least partial knockdown of ATXN3 gene expression, preferably comprising a total knockdown of ATXN3 gene expression.
20. A use in accordance with any one of embodiments 17-19, wherein said use comprises a reduction of ATXN3 protein expression of at least 50%.
21. A use in accordance with any one of embodiments 17-20, wherein said first RNA sequence and said second RNA sequence, when expressed in a cell, are processed by the cell to produce a guide sequence comprising the first RNA sequence, wherein said guide sequences comprise at most 10% of the total miRNA counts as produced by the cell.
22. A use in accordance with any one of embodiments 17-21, wherein said use comprises knockdown of ATXN3 gene expression in the brain stem and/or the cerebellum.
23. A use in accordance with any one of embodiments 17-22, wherein said use comprises improved motor function and/or prolonged survival.
24. A use in accordance with any one of embodiments 17-23, wherein said use comprises a medical treatment of a human subject.

SEQ ID NO 2
GAGAGGGGCAGGGGGCGGAGCTGGAGGGGGTGGTTCGGCGTGGGGGCCGT
TGGCTCCAGACAAATAAACATGGAGTCCATCTTCCACGAGAAACAAGAAG
GCTCACTTTGTGCTCAACATTGCCTGAATAACTTATTGCAAGGAGAATAT
TTTAGCCCTGTGGAATTATCCTCAATTGCACATCAGCTGGATGAGGAGGA
GAGGATGAGAATGGCAGAAGGAGGAGTTACTAGTGAAGATTATCGCACGT
TTTTACAGCAGCCTTCTGGAAATATGGATGACAGTGGTTTTTTCTCTATT
CAGGTTATAAGCAATGCCTTGAAAGTTTGGGGTTTAGAACTAATCCTGTT
CAACAGTCCAGAGTATCAGAGGCTCAGGATCGATCCTATAAATGAAAGAT
CATTTATATGCAATTATAAGGAACACTGGTTTACAGTTAGAAAATTAGGA
AAACAGTGGTTTAACTTGAATTCTCTCTTGACGGGTCCAGAATTAATATC
AGATACATATCTTGCACTTTTCTTGGCTCAATTACAACAGGAAGGTTATT
CTATATTTGTCGTTAAGGGTGATCTGCCAGATTGCGAAGCTGACCAACTC
CTGCAGATGATTAGGGTCCAACAGATGCATCGACCAAAACTTATTGGAGA
AGAATTAGCACAACTAAAAGAGCAAAGAGTCCATAAAACAGACCTGGAAC
GAGTGTTAGAAGCAAATGATGGCTCAGGAATGTTAGACGAAGATGAGGAG
GATTTGCAGAGGGCTCTGGCACTAAGTCGCCAAGAAATTGACATGGAAGA
TGAGGAAGCAGATCTCCGCAGGGCTATTCAGCTAAGTATGCAAGGTAGTT
CCAGAAACATATCTCAAGATATGACACAGACATCAGGTACAAATCTTACT
TCAGAAGAGCTTCGGAAGAGACGAGAAGCCTACTTTGAAAAACAGCAGCA
AAAGCAGCAACAGCAGCAGCAGCAGCAGCAGCAGGGGGACCTATCAGGAC
AGAGTTCACATCCATGTGAAAGGCCAGCCACCAGTTCAGGAGCACTTGGG
AGTGATCTAGGTGATGCTATGAGTGAAGAAGACATGCTTCAGGCAGCTGT
GACCATGTCTTTAGAAACTGTCAGAAATGATTTGAAAACAGAAGGAAAAA
AATAATACCTTTAAAAAATAATTTAGATATTCATACTTTCCAACATTATC
CTGTGTGATTACAGCATAGGGTCCACTTTGGTAATGTGTCAAAGAGATGA
GGAAATAAGACTTTTAGCGGTTTGCAAACAAAATGATGGGAAAGTGGAAC
AATGCGTCGGTTGTAGGACTAAATAATGATCTTCCAAATATTAGCCAAAG
AGGCATTCAGCAATTAAAGACATTTAAAATAGTTTTCTAAATGTTTCTTT
TTCTTTTTTGAGTGTGCAATATGTAACATGTCTAAAGTTAGGGCATTTTT
CTTGGATCTTTTTGCAGACTAGCTAATTAGCTCTCGCCTCAGGCTTTTTC
CATATAGTTTGTTTTCTTTTTCTGTCTTGTAGGTAAGTTGGCTCACATCA
TGTAATAGTGGCTTTCATTTCTTATTAACCAAATTAACCTTTCAGGAAAG
TATCTCTACTTTCCTGATGTTGATAATAGTAATGGTTCTAGAAGGATGAA
CAGTTCTCCCTTCAACTGTATACCGTGTGCTCCAGTGTTTTCTTGTGTTG
TTTTCTCTGATCACAACTTTTCTGCTACCTGGTTTTCATTATTTTCCCAC
AATTCTTTTGAAAGATGGTAATCTTTTCTGAGGTTTAGCGTTTTAAGCCC
TACGATGGGATCATTATTTCATGACTGGTGCGTTCCTAAACTCTGAAATC
AGCCTTGCACAAGTACTTGAGAATAAATGAGCATTTTTTAAAATGTGTGA
GCATGTGCTTTCCCAGATGCTTTATGAATGTCTTTTCACTTATATCAAAA
CCTTACAGCTTTGTTGCAACCCCTTCTTCCTGCGCCTTATTTTTTCCTTT
CTTCTCCAATTGAGAAAACTAGGAGAAGCATAGTATGCAGGCAAGTCTCC
TTCTGTTAGAAGACTAAACATACGTACCCACCATGAATGTATGATACATG
AAATTTGGCCTTCAATTTTAATAGCAGTTTTATTTTATTTTTTCTCCTAT
GACTGGAGCTTTGTGTTCTCTTTACAGTTGAGTCATGGAATGTAGGTGTC
TGCTTCACATCTTTTAGTAGGTATAGCTTGTCAAAGATGGTGATCTGGAA
CATGAAAATAATTTACTAATGAAAATATGTTTAAATTTATACTGTGATTT
GACACTTGCATCATGTTTAGATAGCTTAAGAACAATGGAAGTCACAGTAC
TTAGTGGATCTATAAATAAGAAAGTCCATAGTTTTGATAAATATTCTCTT
TAATTGAGATGTACAGAGAGTTTCTTGCTGGGTCAATAGGATAGTATCAT
TTTGGTGAAAACCATGTCTCTGAAATTGATGTTTTAGTTTCAGTGTTCCC
TATCCCTCATTCTCCATCTCCTTTTGAAGCTCTTTTGAATGTTGAATTGT
TCATAAGCTAAAATCCAAGAAATTTCAGCTGACAACTTCGAAAATTATAA
TATGGTATATTGCCCTCCTGGTGTGTGGCTGCACACATTTTATCAGGGAA
AGTTTTTTGATCTAGGATTTATTGCTAACTAACTGAAAAGAGAAGAAAAA
ATATCTTTTATTTATGATTATAAAATAGCTTTTTCTTCGATATAACAGAT
TTTTTAAGTCATTATTTTGTGCCAATCAGTTTTCTGAAGTTTCCCTTACA
CAAAAGGATAGCTTTATTTTAAAATCTAAAGTTTCTTTTAATAGTTAAAA
ATGTTTCAGAAGAATTATAAAACTTTAAAACTGCAAGGGATGTTGGAGTT
TAGTACTACTCCCTCAAGATTTAAAAAGCTAAATATTTTAAGACTGAACA
TTTATGTTAATTATTACCAGTGTGTTTGTCATATTTTCCATGGATATTTG
TTCATTACCTTTTTCCATTGAAAAGTTACATTAAACTTTTCATACACTTG
AATTGATGAGCTACCTAATATAAAAATGAGAAAACCAATATGCATTTTAA
AGTTTTAACTTTAGAGTTTATAAAGTTCATATATACCCTAGTTAAAGCAC
TTAAGAAAATATGGCATGTTTGACTTTTAGTTCCTAGAGAGTTTTTGTTT
TTGTTTTTGTTTTTTTTTGAGACGGAGTCTTGCTATGTCTCCCAGGCTGG
AGGGCAGTGGCATGATCTCGGCTCACTACAACTTCCACCTCCCGGGTTCA
AGCAATTCTCCTGCCTCAGCCTCCAGAGTAGCTGGGATTACAGGCGCCCA
CCACCACACCCGGCAGATTTTTGTATTTTTGGTAGAGACGCGGTTTCATC
ATGTTTGGCCAGGCTGGTCTCGAACTCCTGACCTCAGGTGATCCGCCTGC
CTTGGCCTCCCAAAGTGTTGGGATTACAGGCATGAGCCACTGCGCCTGGC
CAGCTAGAGAGTTTTTAAAGCAGAGCTGAGCACACACTGGATGCGTTTGA
ATGTGTTTGTGTAGTTTGTTGTGAAATTGTTACATTTAGCAGGCAGATCC
AGAAGCACTAGTGAACTGTCATCTTGGTGGGGTTGGCTTAAATTTAATTG
ACTGTTTAGATTCCATTTCTTAATTGATTGGCCAGTATGAAAAGATGCCA
GTGCAAGTAACCATAGTATCAAAAAAGTTAAAAATTATTCAAAGCTATAG
TTTATACATCAGGTACTGCCATTTACTGTAAACCACCTGCAAGAAAGTCA
GGAACAACTAAATTCACAAGAACTGTCCTGCTAAGAAGTGTATTAAAGAT
TTCCATTTTGTTTTACTAATTGGGAACATCTTAATGTTTAATATTTAAAC
TATTGGTATCATTTTTCTAATGTATAATTTGTATTACTGGGATCAAGTAT
GTACAGTGGTGATGCTAGTAGAAGTTTAAGCCTTGGAAATACCACTTTCA
TATTTTCAGATGTCATGGATTTAATGAGTAATTTATGTTTTTAAAATTCA
GAATAGTTAATCTCTGATCTAAAACCATCAATCTATGTTTTTTACGGTAA
TCATGTAAATATTTCAGTAATATAAACTGTTTGAAAAGGCTGCTGCAGGT
AAACTCTATACTAGGATCTTGGCCAAATAATTTACAATTCACAGAATATT
TTATTTAAGGTGGTGCTTTTTTTTTTTGTCCTTAAAACTTGATTTTTCTT
AACTTTATTCATGATGCCAAAGTAAATGAGGAAAAAAACTCAAAACCAGT
TGAGTATCATTGCAGACAAAACTACCAGTAGTCCATATTGTTTAATATTA
AGTTGAATAAAATAAATTTTATTTCAGTCAGAGCCTAAATCACATTTTGA
TTGTCTGAATTTTTGATACTATTTTTAAAATCATGCTAGTGGCGGCTGGG
CGTGGTAGCTCACGCCTGTAATCCCAGCATTTTGGGAGGCCGAAGTGGGT
GGATCACGAGGTCGGGAGTTCGAGACCAGCTTGGCCAAAATGGTGAAACC
CCATCTGTACTAAAAACTACAAAAATTAGCTGGGCGCGGTGGCAGGTGCC
TGTAATCCCAGCTACCTGGGAGTCTGAGGCAGGAGAATTGCTTGAACCCT
GGCGACAGAGGATGCAGTGAGCCAAGATGGTGCCACTGTACTCCAGACTG
GGCGACAGAGTGAGACTCTGTCTCAAAAAAAAAAAAAAAATCATGCTAGT
GCCAAGAGCTACTAAATTCTTAAAACCGGCCCATTGGACCTGTACAGATA
AAAAATAGATTCAGTGCATAATCAAAATATGATAATTTTAAAATCTTAAG
TAGAAAAATAAATCTTGATGTTTTAAATTCTTACGAGGATTCAATAGTTA
ATATTGATGATCTCCCGGCTGGGTGCAGTGGCTCACGCCTGTAATCCCAG
CAGTTCTGGAGGCTGAGGTGGGCGAATCACTTCAGGCCAGGAGTTCAAGA
CCAGTCTGGGCAACATGGTGAAACCTCGTTTCTACTAAAAATACAAAAAT
TAGCCGGGCGTGGTTGCACACACTTGTAATCCCAGCTACTCAGGAGGCTA
AGAATCGCATGAGCCTAGGAGGCAGAGGTTGCAGAGTGCCAAGGGCTCAC
CACTGCATTCCAGCCTGCCCAACAGAGTGAGACACTGTTTCTGAAAAAAA
AAAATATATATATATATATATATATGTGTGTATATATATATGTATATATA
TATGACTTCCTATTAAAAACTTTATCCCAGTCGGGGGCAGTGGCTCACGC
CTGTAATCCCAACACTTTGGGAGGCTGAGGCAGGTGGATCACCTGAAGTC
CGGAGTTTGAGACCAGCCTGGCCAACATGGTGAAACCCCATCTCTACTAA
AAATACAAAACTTAAGCCAGGTATGGTGGCGGGCACCTGTAATCCCAGTT
ACTTGGGAGGCTGAGGCAGGAGAATCGTTTAAACCCAGGAGGTGGAGGTT
GCAGTGAGCTGAGATCGTGCCATTGCACTCTAGCCTGGGCAACAAGAGTA
AAACTCCATCTTAAAGGTTTGTTTGTTTTTTTTTAATCCGGAAACGAAGA
GGCGTTGGGCCGCTATTTTCTTTTTCTTTCTTTCTTTCTTTCTTTTTTTT
TTTTTCTGAGACGGAGTCTAGCTCTGCTGCCCAGGCTGGAGTACAATGAC
ACGATGTTGGCTCACTGCAACCTCCACCTCCTGGGTTCAAGCGATTCTCC
TGCCTCAGCCTCCCAAGTACCTGGGATTACAGGCACCTGCCACTACACCT
GGCGAATATTTGTTTTTTTTAGTAGAGACGGGCTTTTACCATGTTAGGCT
GGTCTCAAACTCCTGACCTCAGGTGATCTGCCTGCCTTGGCCTCCCAAAG
TGCTGGGATTACAGGTGCAGGCCACCACACCCGGCCTTGGGCCACTGTTT
TCAAAGTGAATTGTTTGTTGTATCGAGTCCTTAAGTATGGATATATATGT
GACCCTAATTAAGAACTACCAGATTGGATCAACTAATCATGTCAGCAATG
TAAATAACTTTATTTTTCATATTCAAAATAAAAACTTTCTTTTATTTCTG
GCCCCTTTATAACCAGCATCTTTTTGCTTTAAAAAATGACCTGGCTTTGT
ATTTTTTTAGTCTTAAACATAATAAAAATATTTTTGTTCTAATTTGCTTT
CATGAGTGAAGATTATTGACATCGTTGGTAAATTCTAGAATTTTGATTTT
GTTTTTTAATTTGAAGAAAATCTTTGCTATTATTATTTTTTCCAAGTGGT
CTGGCATTTTAAGAATTAGTGCTAATAACGTAACTTCTAAATTTGTCGTA
ATTGGCATGTTTAATAGCATATCAAAAAACATTTTAAGCCTGTGGATTCA
TAGACAAAGCAATGAGAAACATTAGTAAAATATAAATGGATATTCCTGAT
GCATTTAGGAAGCTCTCAATTGTCTCTTGCATAGTTCAAGGAATGTTTTC
TGAATTTTTTTAATGCTTTTTTTTTTTTTGAAAGAGGAAAACATACATTT
TTAAATGTGATTATCTAATTTTTACAACACTGGGCTATTAGGAATAACTT
TTTAAAAATTACTGTTCTGTATAAATATTTGAAATTCAAGTACAGAAAAT
ATCTGAAACAAAAAGCATTGTTGTTTGGCCATGATACAAGTGCACTGTGG
CAGTGCCGCTTGCTCAGGACCCAGCCCTGCAGCCCTTCTGTGTGTGCTCC
CTCGTTAAGTTCATTTGCTGTTATTACACACACAGGCCTTCCTGTCTGGT
CGTTAGAAAAGCCGGGCTTCCAAAGCACTGTTGAACACAGGATTCTGTTG
TTAGTGTGGATGTTCAATGAGTTGTATTTTAAATATCAAAGATTATTAAA
TAAAGATAATGTTTGCTTTTCTA

1. An expression cassette encoding a double stranded RNA comprising a first RNA sequence and a second RNA sequence wherein the first and second RNA sequence are substantially complementary, wherein the first RNA sequence has a sequence length of at least 19 nucleotides and is substantially complementary to a target RNA sequence comprised in an RNA encoded by a human ATXN3 gene.

2. An expression cassette according to claim 1, wherein said target RNA sequence is comprised in the region 5′ to the RNA sequence encoded by the sequence corresponding with nucleotides 942-1060 of SEQ ID NO. 2 of the human ATXN3 gene.

3. An expression cassette according to claim 2, wherein said target RNA sequence is comprised in the RNA sequence encoded by the region 390-456 of SEQ ID NO.2 and sequences 3′ therefrom.

4. An expression cassette according to claim 2, wherein said target RNA sequence is selected from the group consisting of consisting of SEQ ID NOs. 9-13.

5. An expression cassette according to claim 1, wherein said first and second RNA sequence are comprised in a pre-miRNA scaffold, a pri-miRNA scaffold or a shRNA.

6. An expression cassette according to claim 5, wherein said pre-miRNA scaffold or said pri-miRNA scaffold is from miR451.

7. An expression cassette according to claim 3, wherein the first RNA sequence is selected from the group consisting of SEQ ID NOs. 14-17.

8. An expression cassette according to claim 7 wherein the first RNA sequence and second RNA sequence are selected from the group consisting of the combinations of SEQ ID NOs. 14 and 18; SEQ ID NOs. 15 and 19; SEQ ID NOs. 16 and 20; SEQ ID NOs. 17 and 21.

9. An expression cassette according to claim 8, wherein said encoded RNA comprises an RNA sequence selected from the group consisting of SEQ ID NOs. 22-29.

10. An expression cassette according to claim 1, wherein the expression cassette comprises a PGK promoter, a CMV promoter, a neuron-specific promoter, a astrocyte-specific promoter or a CBA promoter operably linked to said nucleic acid sequence encoding said first RNA sequence and said second RNA sequence.

11. A gene therapy vector comprising the expression cassette according to claim 1, wherein said gene therapy vector is an AAV vector.

12. A method of treatment comprising administering a gene therapy vector according to claim 11 to a patient in need thereof.

13. The method of claim 12, wherein the treatment is for SCA3/MJD.

14. The method of claim 12, wherein the treatment comprises total knockdown of ATXN3 gene expression.

15. The method of claim 12, wherein the treatment comprises knockdown of ATXN3 gene expression in the brain stem and/or the cerebellum.
